(19) United States
(12) Patent Application Publication
(10) Pub. No.: US 2017/0206335 A1
(43) Pub. Date: Jul. 20, 2017

(54) EVALUATING METHOD, EVALUATING APPARATUS, EVALUATING PROGRAM PRODUCT, EVALUATING SYSTEM, AND TERMINAL APPARATUS

(71) Applicant: AJINOMOTO CO., INC., Tokyo (JP)

(72) Inventors: Kenji NAGAO, Kanagawa (JP); Akira IMAIZUMI, Kanagawa (JP); Minoru YAMAKADO, Tokyo (JP)

(73) Assignee: AJINOMOTO CO., INC., Tokyo (JP)

(21) Appl. No.: 15/479,786

(22) Filed: Apr. 5, 2017

Related U.S. Application Data
(63) Continuation of application No. PCT/JP2015/078674, filed on Oct. 8, 2015.

(30) Foreign Application Priority Data
Oct. 8, 2014 (JP) 2014-207599
Jun. 12, 2015 (JP) 2015-119722

(51) Int. Cl. G06F 19/00; G01N 33/68

(52) U.S. Cl. CPC G06F 19/3431; G01N 33/6806; G01N 2800/50; G01N 2800/2871; G01N 2800/326; G01N 2800/22

(57) ABSTRACT

An evaluating method includes an evaluating step of evaluating a future risk of lifestyle-related disease for a subject to be evaluated using a concentration value of an amino acid included in amino acid concentration data on the concentration value of the amino acid in blood collected from the subject.

### CROSS-REFERENCE TO RELATED APPLICATION

This application is based upon and claims the benefit of priority from PCT Application PCT/JP2015/078674, filed Oct. 8, 2015, which claims priority from Japanese Patent Application No. 2014-207599, filed Oct. 8, 2014 and Japanese Patent Application No. 2015-119722, filed Jun. 12, 2015, the entire contents of which are incorporated herein by reference.

### BACKGROUND OF THE INVENTION

1. Field of the Invention

The present invention relates to a method of evaluating future risk of lifestyle-related disease, an evaluating apparatus, an evaluating program product, an evaluating system, and a terminal apparatus.

2. Description of the Related Art

Biomarker testing has been rapidly developed with the recent progress of genome analysis and post-genome testing and is widely utilized for, for example, prevention, diagnosis, and prognosis estimation of diseases. Examples of biomarkers studies testing of which is actively performed include genomics and transcriptomics based on gene information, proteomics based on protein information, and metabolomics based on metabolite information.

Genomics and transcriptomics reflect genetic factors, but have a problem of not reflecting environmental factors. Proteomics requires analysis of many kinds of proteins, and still has many problems in analytical methods and comprehensive analysis methods. Metabolomics is promising in that it is a biomarker that reflects environmental factors in addition to genetic factors but, because of a large number of metabolites, still has many problems in comprehensive analysis methods.

In view of this, amino acids, which play a dominant role in metabolic pathways among metabolites in living bodies, are drawing attention as a novel biomarker.

It is reported that amino acid concentrations vary with diseases such as liver failure and renal failure (“Rosen H M, Yoshimura N, Hodgman J M, et al., “Plasma amino acid patterns in hepatic encephalopathy of differing etiology”, Gastroenterology, 1977, 72, 483-487″ and “Suliman M E, Qureshi A R, Stenvinkel P, et al., “Inflammation contributes to low plasma amino acid concentrations in patients with chronic kidney disease”, Am. J. Clin. Nutr., 2005, 82, 342-349″).

WO 2004/052191, WO 2006/098192, and WO 2009/054351 related to a method of relating an amino acid concentration to a biological state are disclosed as previous patents. As prior patents, WO 2008/015929 related to a method of evaluating a state of metabolic syndrome using an amino acid concentration, WO 2009/001862 related to a method of evaluating a state of visceral fat accumulation using an amino acid concentration, WO 2009/054350 related to a method of evaluating a state of impaired glucose tolerance using an amino acid concentration, WO 2010/095682 related to a method of evaluating a state of at least one of apparent obesity, non-apparent obesity, and obesity that are defined by body mass index (BMI) and visceral fat area (VFA) using an amino acid concentration, WO 2013/002381 related to a method of evaluating a state of fatty liver related disease including at least one of fatty liver, non-alcoholic fatty liver disease (NAFLD), and non-alcoholic steatohepatitis (NASH) using an amino acid concentration, WO 2013/115283 related to a method of evaluating a state of early-stage nephropathy (for example, whether early-stage nephropathy develops in the future) using an amino acid concentration, and JP-A-2013-178238 related to a method of evaluating a future state of a cardiovascular event using an amino acid concentration are disclosed.

However, no search has been conducted for amino acids that are clinically useful for evaluating the states of indicators of lifestyle-related diseases (for example, the risk factors of lifestyle-related diseases that may be caused mainly by metabolic syndrome (for example, visceral fat accumulation, insulin resistance, and fatty liver)) in light of preventive medicine. Hence, no method has been developed of accurately and systematically evaluating the states of indicators of lifestyle-related diseases using amino acid concentrations. For example, although it is known that the progress of metabolic syndrome causes serious diseases such as cardiovascular events and cerebrovascular events in the future, no search has been conducted for a method of preventing these events using the profiles of amino acids in blood (see “Despres J P, Lemieux I, “Abdominal obesity and metabolic syndrome”, Nature, 2006, 444, 881-887″ and “Van Gaal L F, Mertens I L, DeBlock C E, “Mechanisms linking obesity with cardiovascular disease”, Nature, 2006, 444, 875-880”).

In biological state evaluation using an amino acid concentration in blood described in WO 2004/052191, WO 2006/098192, WO 2009/054351, WO 2008/015929, WO 2009/001862, WO 2009/054350, WO 2010/095682, WO 2013/002381, WO 2013/115283, and JP-A-2013-178238, examples of utilizing information on amino acids that are useful for evaluating the states of indicators of lifestyle-related diseases and the clinical significance of which is great are given, but there is a problem in that information on behavior of each amino acid is lost when information on a plurality of amino acids behavior of which differ between individuals is one-dimensionally compressed. Thus, based on the behavior of an amino acid concentration in blood for each individual, events of serious diseases such as cardiovascular events and cerebrovascular events caused by, for example, the progress of metabolic syndrome in the future need to be predicted.

### SUMMARY OF THE INVENTION

It is an object of the present invention to at least partially solve the problems in the conventional technology.

The present invention has been made in view of the problems described above, and an object of the present invention is to provide an evaluating method, an evaluating apparatus, an evaluating program product, an evaluating system, and a terminal apparatus, which can provide reliable information that may be helpful in knowing a future risk of lifestyle-related disease.

To solve the problem and achieve the object described above, an evaluating method according to one aspect of the present invention includes an evaluating step of evaluating a future risk of lifestyle-related disease for a subject to be evaluated using a concentration value of an amino acid included in amino acid concentration data on the concentration value of the amino acid in blood collected from the subject.

In the present specification, various amino acids are mainly written in abbreviations, the formal names of these are as follows.

| (Abbreviation) | (Formal name) |
|---|---|
| a-ABA | a-Aminobutyric acid |
| Ala | Alanine |
| Arg | Arginine |
| Asn | Asparagine |
| Cit | Citrulline |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Orn | Ornithine |
| Phe | Phenylalanine |
| Pro | Proline |
| Ser | Serine |
| Thr | Threonine |
| Trp | Tryptophan |
| Tyr | Tyrosine |
| Val | Valine |

Essential amino acids are His, Ile, Leu, Lys, Met, Phe, Thr, Trp, and Val. A semiessential amino acid is Arg, and examples thereof may further include cysteine (Cys) and Tyr.

In the present invention, lifestyle-related disease refers to a group of diseases onset and progress of which are associated with lifestyle including dietary habit, exercise habit, rest, smoking, drinking, and the like. Examples thereof include hypertension, fatty liver, high-risk fatty liver, diabetes, impaired glucose tolerance, obesity, morbid obesity, dyslipidemia, chronic nephropathy, arteriosclerosis, cerebral infarction, heart disease, metabolic syndrome, sympathetic nerve disorder, inflammatory disease, anemia, protein malnutrition, immune depression, obese physique, respiratory disease, cardiovascular disease, high blood pressure, kidney and urinary tract disease, gastrointestinal disease, liver disease, biliopancreatic disease, carbohydrate metabolism disorder, lipid metabolism disorder, uric acid metabolism disorder, blood disease, serum disease, eye disease, hearing abnormality, urinary system disease, high tumor marker levels, gynecologic disease, breast disease, encephalopathy, reduction in bone mineral density, atrial fibrillation, and arrhythmia.

The evaluating method according to another aspect of the present invention is the evaluating method, wherein at the evaluating step, if the concentration value of an amino acid included in the amino acid concentration data or a converted value of the concentration value is smaller than a predetermined value or is equal to or smaller than the predetermined value, or the concentration value or the converted value is equal to or greater than a predetermined value or is greater than the predetermined value, the future risk of lifestyle-related disease for the subject is evaluated.

The evaluating method according to still another aspect of the present invention is the evaluating method, wherein the amino acid concentration data includes concentration values of His, Ile, Leu, Lys, Met, Phe, Thr, Trp, Val, and Arg.

The evaluating method according to still another aspect of the present invention is the evaluating method, wherein at the evaluating step, if a concentration value or a converted value of the concentration value of at least one amino acid of His, Ile, Leu, Lys, Met, Phe, Thr, Trp, Val, and Arg is smaller than a predetermined value or is equal to or smaller than the predetermined value, or the concentration value or the converted value is equal to or greater than a predetermined value or is greater than the predetermined value, a future risk of developing at least one of cerebral infarction, anemia, atrial fibrillation, and arrhythmia for the subject is evaluated.

The evaluating method according to still another aspect of the present invention is the evaluating method, wherein at the evaluating step, at least one of (1) evaluation of a future risk of developing anemia if a concentration value or a converted value of the concentration value of at least one amino acid of Lys, Leu, and Trp is smaller than a predetermined value or is equal to or smaller than the predetermined value, (2) evaluation of a future risk of developing cerebral infarction if a concentration value or a converted value of the concentration value of at least one amino acid of His, Met, and Phe is smaller than a predetermined value or is equal to or smaller than the predetermined value, and (3) evaluation of a future risk of developing any one or both of atrial fibrillation and arrhythmia if a concentration value or a converted value of the concentration value of Thr or Arg is smaller than a predetermined value or is equal to or smaller than the predetermined value is performed.

The evaluating method according to still another aspect of the present invention is the evaluating method, wherein the converted value is an amino acid concentration standard score that is a value into which a concentration value of an amino acid is standardized, and the amino acid concentration standard score is used at the evaluating step.

An evaluating apparatus according to one aspect of the present invention is an evaluating apparatus including a control unit. The control unit includes an evaluating unit that evaluates a future risk of lifestyle-related disease for a subject to be evaluated using a concentration value of an amino acid included in amino acid concentration data of the subject on the concentration value of the amino acid in blood.

An evaluating method according to one aspect of the present invention is an evaluating method carried out with an information processing apparatus including a control unit. The evaluating method includes an evaluating step of evaluating a future risk of lifestyle-related disease for a subject to be evaluated using a concentration value of an amino acid included in amino acid concentration data of the subject on the concentration value of the amino acid in blood. The evaluating step is executed by the control unit.

An evaluating program product according to one aspect of the present invention is an evaluating program product having a non-transitory computer readable medium including programmed instructions for making an information processing apparatus including a control unit execute an evaluating method. The evaluating method includes an evaluating step of evaluating a future risk of lifestyle-related disease for a subject to be evaluated using a concentration value of an amino acid included in amino acid concentration data of the subject on the concentration value of the amino acid in blood. The evaluating step is executed by the control unit.

A recording medium according to one aspect of the present invention is a non-transitory computer-readable recording medium including the programmed instructions for making an information processing apparatus execute the evaluating method.

An evaluating system according to one aspect of the present invention is an evaluating system including (I) an evaluating apparatus including a control unit and (II) a terminal apparatus including a control unit to provide amino acid concentration data of a subject to be evaluated on a concentration value of an amino acid in blood that are connected to each other communicatively via a network. The control unit of the terminal apparatus includes (I) an amino acid concentration data-sending unit that transmits the amino acid concentration data of the subject to the evaluating apparatus and (II) a result-receiving unit that receives an evaluation result on a future risk of lifestyle-related disease for the subject, transmitted from the evaluating apparatus. The control unit of the evaluating apparatus includes (I) an amino acid concentration data-receiving unit that receives the amino acid concentration data of the subject transmitted from the terminal apparatus, (II) an evaluating unit that evaluates the future risk of lifestyle-related disease for the subject using the concentration value of the amino acid included in the amino acid concentration data of the subject received by the amino acid concentration data-receiving unit, and (III) a result-sending unit that transmits the evaluation result obtained by the evaluating unit to the terminal apparatus.

A terminal apparatus according to one aspect of the present invention is a terminal apparatus including a control unit. The control unit includes a result-obtaining unit that obtains an evaluation result on a future risk of lifestyle-related disease for a subject to be evaluated. The evaluation result is the result of evaluating the future risk of lifestyle-related disease for the subject using a concentration value of an amino acid included in amino acid concentration data of the subject on the concentration value of the amino acid in blood.

The terminal apparatus according to another aspect of the present invention is the terminal apparatus, wherein the apparatus is communicatively connected via a network to an evaluating apparatus that evaluates the future risk of lifestyle-related disease for the subject. The control unit further includes an amino acid concentration data-sending unit that transmits the amino acid concentration data of the subject to the evaluating apparatus. The result-obtaining unit receives the evaluation result transmitted from the evaluating apparatus.

An evaluating apparatus according to one aspect of the present invention is an evaluating apparatus including a control unit, being connected communicatively via a network to a terminal apparatus that provides amino acid concentration data of a subject to be evaluated on a concentration value of an amino acid in blood. The control unit includes (I) an amino acid concentration data-receiving unit that receives the amino acid concentration data of the subject transmitted from the terminal apparatus, (II) an evaluating unit that evaluates a future risk of lifestyle-related disease for the subject using the concentration value of the amino acid included in the amino acid concentration data of the subject received by the amino acid concentration data-receiving unit, and (III) a result-sending unit that transmits an evaluation result obtained by the evaluating unit to the terminal apparatus.

According to the present invention, the concentration value of the amino acid included in the amino acid concentration data on the concentration value of the amino acid in blood collected from the subject is used to evaluate the future risk of lifestyle-related disease for the subject. Thus, the present invention achieves the effect of being able to provide reliable information that may be helpful in knowing the future risk of lifestyle-related disease.

According to the present invention, by evaluating the future risk of lifestyle-related disease (degree of the possibility of developing lifestyle-related disease in the future), the risk can be known at preclinical stages of lifestyle-related disease or at earlier stages of lifestyle-related disease, which leads to prevention of lifestyle-related disease.

According to the present invention, by considering the concentration value of the amino acid in blood, suggestions (e.g., menu suggestions including any one, some, or all of intake of drug, amino acid, food, supplement, and the like, a diet, and exercise or the like) can be made to reduce the future risk of lifestyle-related disease.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of a first embodiment;

FIG. 2 is a principle configurational diagram showing a basic principle of a second embodiment;

FIG. 3 is a diagram showing an example of an entire configuration of a present system;

FIG. 4 is a diagram showing another example of an entire configuration of the present system;

FIG. 5 is a block diagram showing an example of a configuration of an evaluating apparatus 100 in the present system;

FIG. 6 is a chart showing an example of information stored in a user information file 106a;

FIG. 7 is a chart showing an example of information stored in an amino acid concentration data file 106b;

FIG. 8 is a chart showing an example of information stored in an index state information file 106c;

FIG. 9 is a chart showing an example of information stored in a designated index state information file 106d;

FIG. 10 is a chart showing an example of information stored in a candidate formula file 106e1;

FIG. 11 is a chart showing an example of information stored in a verification result file 106e2;

FIG. 12 is a chart showing an example of information stored in a selected index state information file 106e3;

FIG. 13 is a chart showing an example of information stored in an evaluation formula file 106e4;

FIG. 14 is a chart showing an example of information stored in an evaluation result file 106f;

FIG. 15 is a block diagram showing a configuration of an evaluation formula-preparing part 102h;

FIG. 16 is a block diagram showing a configuration of an evaluating part 102i;

FIG. 17 is a block diagram showing an example of a configuration of a client apparatus 200 in the present system;

FIG. 18 is a block diagram showing an example of a configuration of a database apparatus 400 in the present system;

FIG. 19 is a flowchart showing an example of an evaluation formula-preparing processing performed in the evaluating apparatus 100 in the present system;

FIG. 20 is a table of odds ratios for non-adjusted background factors;

FIG. 21 is a table of odds ratios for non-adjusted background factors;

FIG. 22 is a table of odds ratios for non-adjusted background factors;

FIG. 23 is a table of odds ratios for non-adjusted background factors;

FIG. 24 is a table of odds ratios for non-adjusted background factors;

FIG. 25 is a table of odds ratios for non-adjusted background factors;

FIG. 26 is a table of odds ratios for non-adjusted background factors;

FIG. 27 is a table of odds ratios for non-adjusted background factors;

FIG. 28 is a table of odds ratios for non-adjusted background factors;

FIG. 29 is a table of odds ratios for non-adjusted background factors;

FIG. 30 is a table of odds ratios for non-adjusted background factors;

FIG. 31 is a table of odds ratios for non-adjusted background factors;

FIG. 32 is a table of odds ratios for non-adjusted background factors;

FIG. 33 is a table of odds ratios for non-adjusted background factors;

FIG. 34 is a table of odds ratios for non-adjusted background factors;

FIG. 35 is a table of gender-adjusted odds ratios;

FIG. 36 is a table of gender-adjusted odds ratios;

FIG. 37 is a table of gender-adjusted odds ratios;

FIG. 38 is a table of gender-adjusted odds ratios;

FIG. 39 is a table of gender-adjusted odds ratios;

FIG. 40 is a table of gender-adjusted odds ratios;

FIG. 41 is a table of gender-adjusted odds ratios;

FIG. 42 is a table of gender-adjusted odds ratios;

FIG. 43 is a table of gender-adjusted odds ratios;

FIG. 44 is a table of gender-adjusted odds ratios;

FIG. 45 is a table of gender-adjusted odds ratios;

FIG. 46 is a table of gender-adjusted odds ratios;

FIG. 47 is a table of gender-adjusted odds ratios;

FIG. 48 is a table of gender-adjusted odds ratios;

FIG. 49 is a table of gender-adjusted odds ratios;

FIG. 50 is a table of age-adjusted odds ratios;

FIG. 51 is a table of age-adjusted odds ratios;

FIG. 52 is a table of age-adjusted odds ratios;

FIG. 53 is a table of age-adjusted odds ratios;

FIG. 54 is a table of age-adjusted odds ratios;

FIG. 55 is a table of age-adjusted odds ratios;

FIG. 56 is a table of age-adjusted odds ratios;

FIG. 57 is a table of age-adjusted odds ratios;

FIG. 58 is a table of age-adjusted odds ratios;

FIG. 59 is a table of age-adjusted odds ratios;

FIG. 60 is a table of age-adjusted odds ratios;

FIG. 61 is a table of age-adjusted odds ratios;

FIG. 62 is a table of age-adjusted odds ratios;

FIG. 63 is a table of age-adjusted odds ratios;

FIG. 64 is a table of BMI-adjusted odds ratios;

FIG. 65 is a table of BMI-adjusted odds ratios;

FIG. 66 is a table of BMI-adjusted odds ratios;

FIG. 67 is a table of BMI-adjusted odds ratios;

FIG. 68 is a table of BMI-adjusted odds ratios;

FIG. 69 is a table of BMI-adjusted odds ratios;

FIG. 70 is a table of BMI-adjusted odds ratios;

FIG. 71 is a table of BMI-adjusted odds ratios;

FIG. 72 is a table of BMI-adjusted odds ratios;

FIG. 73 is a table of BMI-adjusted odds ratios;

FIG. 74 is a table of BMI-adjusted odds ratios;

FIG. 75 is a table of gender and age-adjusted odds ratios;

FIG. 76 is a table of gender and age-adjusted odds ratios;

FIG. 77 is a table of gender and age-adjusted odds ratios;

FIG. 78 is a table of gender and age-adjusted odds ratios;

FIG. 79 is a table of gender and age-adjusted odds ratios;

FIG. 80 is a table of gender and age-adjusted odds ratios;

FIG. 81 is a table of gender and age-adjusted odds ratios;

FIG. 82 is a table of gender and age-adjusted odds ratios;

FIG. 83 is a table of gender and age-adjusted odds ratios;

FIG. 84 is a table of gender and age-adjusted odds ratios;

FIG. 85 is a table of gender and age-adjusted odds ratios;

FIG. 86 is a table of gender and age-adjusted odds ratios;

FIG. 87 is a table of gender and age-adjusted odds ratios;

FIG. 88 is a table of gender and age-adjusted odds ratios;

FIG. 89 is a table of gender and BMI-adjusted odds ratios;

FIG. 90 is a table of gender and BMI-adjusted odds ratios;

FIG. 91 is a table of gender and BMI-adjusted odds ratios;

FIG. 92 is a table of gender and BMI-adjusted odds ratios;

FIG. 93 is a table of gender and BMI-adjusted odds ratios;

FIG. 94 is a table of gender and BMI-adjusted odds ratios;

FIG. 95 is a table of gender and BMI-adjusted odds ratios;

FIG. 96 is a table of gender and BMI-adjusted odds ratios;

FIG. 97 is a table of gender and BMI-adjusted odds ratios;

FIG. 98 is a table of gender and BMI-adjusted odds ratios;

FIG. 99 is a table of gender and BMI-adjusted odds ratios;

FIG. 100 is a table of age and BMI-adjusted odds ratios;

FIG. 101 is a table of age and BMI-adjusted odds ratios;

FIG. 102 is a table of age and BMI-adjusted odds ratios;

FIG. 103 is a table of age and BMI-adjusted odds ratios;

FIG. 104 is a table of age and BMI-adjusted odds ratios;

FIG. 105 is a table of age and BMI-adjusted odds ratios;

FIG. 106 is a table of age and BMI-adjusted odds ratios;

FIG. 107 is a table of age and BMI-adjusted odds ratios;

FIG. 108 is a table of age and BMI-adjusted odds ratios;

FIG. 109 is a table of gender, age, and BMI-adjusted odds ratios;

FIG. 110 is a table of gender, age, and BMI-adjusted odds ratios;

FIG. 111 is a table of gender, age, and BMI-adjusted odds ratios;

FIG. 112 is a table of gender, age, and BMI-adjusted odds ratios;

FIG. 113 is a table of gender, age, and BMI-adjusted odds ratios;

FIG. 114 is a table of gender, age, and BMI-adjusted odds ratios;

FIG. 115 is a table of gender, age, and BMI-adjusted odds ratios;

FIG. 116 is a table of gender, age, and BMI-adjusted odds ratios;

FIG. 117 is a table of gender, age, and BMI-adjusted odds ratios;

FIG. 118 is a table of results indicating whether each predetermined condition is satisfied for each combination of amino acid concentration standard scores, values of Index Formulae 1 and 2, and disease events;

FIG. 119 is a table of odds ratios and 95%-confidence intervals thereof for each combination of amino acid concentration standard scores, values of Index Formulae 1 and 2, and disease events;

FIG. 120 is a table of odds ratios and 95%-confidence intervals thereof for each combination of amino acid concentration standard scores, values of Index Formulae 1 and 2, and disease events;

FIG. 121 is a table of odds ratios and 95%-confidence intervals thereof for each combination of amino acid concentration standard scores, values of Index Formulae 1 and 2, and disease events;

FIG. 122 is a table of odds ratios and 95%-confidence intervals thereof for each combination of amino acid concentration standard scores, values of Index Formulae 1 and 2, and disease events;

FIG. 123 is a table of odds ratios and 95%-confidence intervals thereof for each combination of amino acid concentration standard scores, values of Index Formulae 1 and 2, and disease events;

FIG. 124 is a table of odds ratios and 95%-confidence intervals thereof for each combination of amino acid concentration standard scores, values of Index Formulae 1 and 2, and disease events;

FIG. 125 is a table of results indicating whether each predetermined condition is satisfied for each combination of amino acid concentration standard scores corresponding to amino acid low values, amino acid concentration standard scores corresponding to essential amino acid low values, and disease events;

FIG. 126 is a table of odds ratios and 95%-confidence intervals thereof for each combination of amino acid concentration standard scores corresponding to amino acid low values, amino acid concentration standard scores corresponding to essential amino acid low values, and disease events;

FIG. 127 is a table of odds ratios and 95%-confidence intervals thereof for each combination of amino acid concentration standard scores corresponding to amino acid low values, amino acid concentration standard scores corresponding to essential amino acid low values, and disease events;

FIG. 128 is a table of odds ratios and 95%-confidence intervals thereof for each combination of amino acid concentration standard scores corresponding to amino acid low values, amino acid concentration standard scores corresponding to essential amino acid low values, and disease events;

FIG. 129 is a table of results indicating whether each predetermined condition is satisfied for each combination of amino acid concentration standard scores corresponding to amino acid high values, amino acid concentration standard scores corresponding to essential amino acid high values, and disease events;

FIG. 130 is a table of odds ratios and 95%-confidence intervals thereof for each combination of amino acid concentration standard scores corresponding to amino acid high values, amino acid concentration standard scores corresponding to essential amino acid high values, and disease events;

FIG. 131 is a table of odds ratios and 95%-confidence intervals thereof for each combination of amino acid concentration standard scores corresponding to amino acid high values, amino acid concentration standard scores corresponding to essential amino acid high values, and disease events;

FIG. 132 is a table of odds ratios and 95%-confidence intervals thereof for each combination of amino acid concentration standard scores corresponding to amino acid high values, amino acid concentration standard scores corresponding to essential amino acid high values, and disease events;

FIG. 133A is a table of amino acid sets and odds ratios for each combination of amino acid sets and disease events;

FIG. 133B is a table of amino acid sets and odds ratios for each combination of amino acid sets and disease events;

FIG. 134A is a table of amino acid sets and odds ratios for each combination of amino acid sets and disease events;

FIG. 134B is a table of amino acid sets and odds ratios for each combination of amino acid sets and disease events;

FIG. 135 is a table of amino acid sets and odds ratios for each combination of amino acid sets and disease events;

FIG. 136 is a table of amino acid sets and odds ratios for each combination of amino acid sets and disease events;

FIG. 137 is a table of appearance frequencies and appearance ratios for each amino acid;

FIG. 138A is a table of amino acid sets and odds ratios for each combination of amino acid sets and disease events;

FIG. 138B is a table of amino acid sets and odds ratios for each combination of amino acid sets and disease events;

FIG. 139 is a table of amino acid sets and odds ratios for each combination of amino acid sets and disease events; and

FIG. 140 is a table of appearance frequencies and appearance ratios for each amino acid.

### DETAILED DESCRIPTION OF THE PREFERRED EMBODIMENTS

Hereinafter, an embodiment (first embodiment) of the evaluating method according to the present invention and an embodiment (second embodiment) of the evaluating apparatus, the evaluating method, the evaluating program, the evaluating system, and the terminal apparatus according to the present invention are described in detail with reference to the drawings. The present invention is not limited to these embodiments.

### First Embodiment

1-1. Outline of First Embodiment

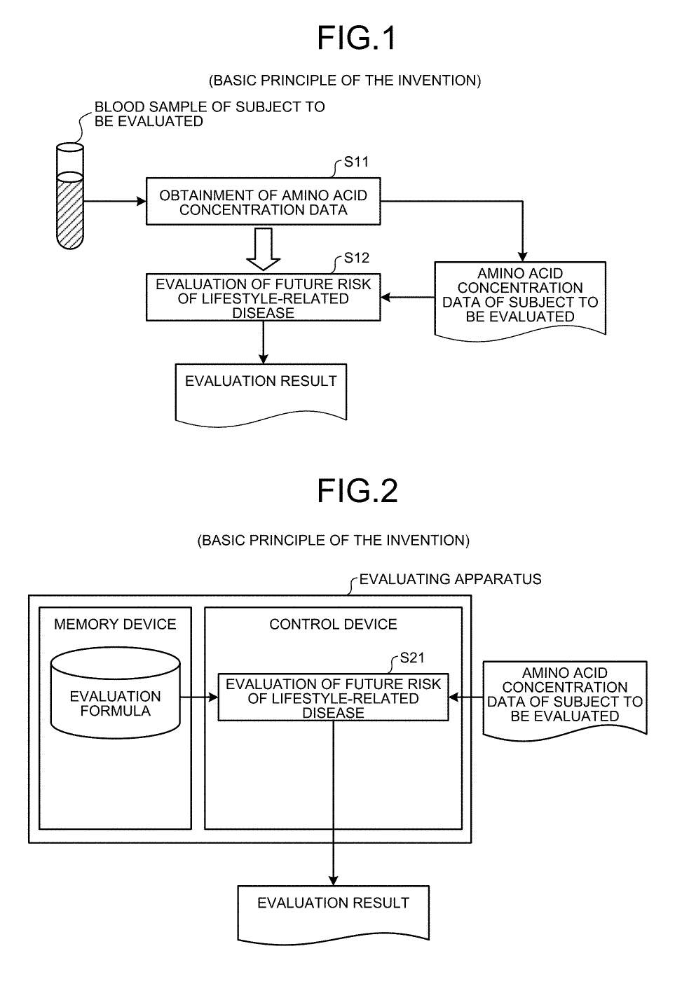

Here, an outline of the first embodiment will be described with reference to FIG. 1. FIG. 1 is a principle configurational diagram showing a basic principle of the first embodiment.

Amino acid concentration data on a concentration value of an amino acid in blood (including, for example, plasma or serum) collected from a subject to be evaluated (for example, an individual such as animal or human) is obtained (step S11).

In step S11, for example, the amino acid concentration data determined by a company or the like that performs amino acid concentration value measurements may be obtained, or the amino acid concentration data may be obtained by determining the concentration value of the amino acid by a measurement method such as, for example, the following method (A), (B), or (C) from blood collected from the subject. Here, the unit of the concentration value of the amino acid may be, for example, a molar concentration, a weight concentration, or one obtained by addition, subtraction, multiplication, and division of any constant with these concentrations.

(A) Plasma is separated from blood by centrifuging a collected blood sample. All plasma samples are frozen and stored at −80° C. until the amino acid concentration value is measured. At the time of measuring the amino acid concentration value, acetonitrile is added to perform a protein removal treatment, pre-column derivatization is then performed using a labeled reagent (3-aminopyridyl-N-hydroxysuccinimidyl carbamate), and an amino acid concentration value is analyzed by liquid chromatograph mass spectrometer (LC/MS) (see International Publication WO 2003/069328 and International Publication WO 2005/116629).

(B) Plasma is separated from blood by centrifuging a collected blood sample. All plasma samples are frozen and stored at −80° C. until the amino acid concentration value is measured. At the time of measuring the amino acid concentration value, sulfosalicylic acid is added to perform a protein removal treatment, and an amino acid concentration value is analyzed by an amino acid analyzer based on post-column derivatization using a ninhydrin reagent.

(C) Blood cell separation is performed on a collected blood sample by using a membrane, MEMS (Micro Electro Mechanical Systems) technology, or the principle of centrifugation, whereby plasma or serum is separated from blood. A plasma or serum sample the concentration value of which is not measured immediately after obtaining the plasma or the serum is frozen and stored at −80° C. until the concentration value is measured. At the time of measuring the concentration value, a molecule or the like that reacts with or binds to a target substance in blood, such as an enzyme or an aptamer, is used to perform quantitative analysis or the like on an increasing or decreasing substance or a spectroscopic value by substrate recognition, whereby the concentration value is analyzed.

A future risk of lifestyle-related disease for the subject is evaluated using, as an evaluation value for evaluating the future risk of lifestyle-related disease, the concentration value of the amino acid included in the amino acid concentration data obtained in step S11 (step S12). Before step S12 is executed, data such as defective and outliers may be removed from the amino acid concentration data obtained in step S11.

According to the first embodiment described above, the amino acid concentration data of the subject is obtained in step S11, and in step S12, the future risk of lifestyle-related disease for the subject is evaluated using, as the evaluation value, the concentration value of the amino acid included in the amino acid concentration data of the subject obtained in step S11. Hence, reliable information that may be helpful in knowing the future risk of lifestyle-related disease can be provided.

It may be decided that at least the concentration value of the amino acid reflects the future risk of lifestyle-related disease for the subject. The concentration value may be converted, for example, by the methods listed below, and it may be decided that the converted value reflects the future risk of lifestyle-related disease for the subject. In other words, the concentration value or the converted value may be treated per se as the evaluation result on the future risk of lifestyle-related disease for the subject.

The concentration value may be converted such that the possible range of the concentration value falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the concentration value, by conversion of the concentration value by a predetermined conversion method (for example, index transformation, logarithm transformation, angular transformation, square root transformation, probit transformation, reciprocal transformation, Box-Cox transformation, or power transformation), or by performing a combination of these computations on the concentration value. For example, the value of an exponential function with the concentration value as an exponent and Napier constant as the base may be further calculated (specifically, the value of p/(1−p) where a natural logarithm ln(p/(1−p)) is equal to the concentration value when the probability p that the future risk of lifestyle-related disease has a predetermined state is defined (for example, a state of exceeding a criterion value)), and a value (specifically, the value of probability p) may be further calculated by dividing the calculated value of the exponential function by the sum of 1 and the value of the exponential function.

The concentration value may be converted such that the converted value is a particular value when a particular condition is met. For example, the concentration value may be converted such that the converted value is 5.0 when the specificity is 80% and the converted value is 8.0 when the specificity is 95%.

For each amino acid, after normally distributing the amino acid concentration distribution, the concentration value may be standardized with a mean of 50 and a standard deviation of 10. This processing may be performed by gender.

The positional information about the position of the predetermined mark (for example, a circle sign or a star sign) corresponding to the concentration value or the converted value may be generated on the predetermined scale (for example, a graduated scale at least marked with graduations corresponding to the upper limit value and the lower limit value in the possible range of the concentration value or the converted value, or part of the range) visually presented on the display device such as the monitor or the physical medium such as paper for evaluating the future risk of lifestyle-related disease, using the concentration value of the amino acid or, if the concentration value is converted, the converted value. Then it may be decided that the generated positional information reflects the future risk of lifestyle-related disease for the subject.

If the amino acid concentration is lower than a predetermined value (mean±1SD, 2SD, 3SD, N quantile, N percentile, or a cutoff value the clinical significance of which is recognized, or the like) or is equal to or lower than the predetermined value, or the amino acid concentration is equal to or higher than the predetermined value or is higher than the predetermined value, the future risk of lifestyle-related disease may be evaluated for the subject. In this case, instead of the amino acid concentration itself, an amino acid concentration standard score (a value obtained by normally distributing the amino acid concentration distribution by gender, and then standardizing the amino acid concentration with a mean of 50 and a standard deviation of 10 for each amino acid) may be used. For example, if the amino acid concentration standard score is lower than the mean−2SD (when the amino acid concentration standard score<30), if the amino acid concentration standard score is higher than the mean+2SD (when the amino acid concentration standard score>70), if the amino acid concentration standard score of at least one of the essential amino acid and the semiessential amino acid is lower than the mean−2SD (when the amino acid concentration standard score<30), or if the amino acid concentration standard score of at least one of the essential amino acid and the semiessential amino acid is higher than the mean+2SD (when the amino acid concentration standard score>70), any one or both of in what lifestyle-related disease a risk exists and to what degree the risk exists may be evaluated for the subject.

The future risk of lifestyle-related disease for the subject may be evaluated by calculating a value of a formula using the concentration value of the amino acid and the formula including an explanatory variable to be substituted with the concentration value of the amino acid. In the present specification, the converted value of the concentration value may be substituted with the explanatory variable to be substituted with the concentration value.

It may be decided that the calculated value of the formula reflects the future risk of lifestyle-related disease for the subject. The value of the formula may be converted, for example, by the methods listed below, and it may be decided that the converted value reflects the future risk of lifestyle-related disease for the subject. In other words, the value of the formula or the converted value may be treated per se as the evaluation result on the future risk of lifestyle-related disease for the subject.

The value of the evaluation formula may be converted such that the possible range of the value of the evaluation formula falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given number with the value of the evaluation formula, by conversion of the value of the evaluation formula by a predetermined conversion method (for example, index transformation, logarithm transformation, angular transformation, square root transformation, probit transformation, reciprocal transformation, Box-Cox transformation, or power transformation), or by performing a combination of these computations on the value of the evaluation formula. For example, the value of an exponential function with the value of the evaluation formula as an exponent and Napier constant as the base may be further calculated (specifically, the value of p/(1−p) where a natural logarithm ln(p/(1−p)) is equal to the value of the evaluation formula when the probability p that the future risk of lifestyle-related disease has a predetermined state is defined (for example, a state of exceeding a criterion value)), and a value (specifically, the value of probability p) may be further calculated by dividing the calculated value of the exponential function by the sum of 1 and the value of the exponential function.

The value of the evaluation formula may be converted such that the converted value is a particular value when a particular condition is met. For example, the value of the evaluation formula may be converted such that the converted value is 5.0 when the specificity is 80% and the converted value is 8.0 when the specificity is 95%.

The value of the evaluation formula may be standardized with a mean of 50 and a standard deviation of 10. This processing may be performed by gender.

The evaluation value in the present specification may be the value of the evaluation formula per se or may be the the converted value of the value of the evaluation formula.

The positional information about the position of the predetermined mark (for example, a circle sign or a star sign) corresponding to the value of the formula or the converted value may be generated on the predetermined scale (for example, a graduated scale at least marked with graduations corresponding to the upper limit value and the lower limit value in the possible range of the value of the formula or the converted value, or part of the range) visually presented on the display device such as the monitor or the physical medium such as paper for evaluating the future risk of lifestyle-related disease, using the value of the formula or, if the value of the formula is converted, the converted value. Then it may be decided that the generated positional information reflects the future risk of lifestyle-related disease for the subject.

The degree of the future risk of lifestyle-related disease in the subject may be qualitatively or quantitatively evaluated.

The subject may be classified into any one of a plurality of categories defined at least considering the degree of the future risk of lifestyle-related disease, using “the concentration value of the amino acid and the one or more preset thresholds” or “the concentration value of the amino acid, the formula including the explanatory variable to be substituted with the concentration value of the amino acid, and the one or more preset thresholds”. The categories may include (i) a category to which a subject whose future risk of lifestyle-related disease (the degree of the possibility of developing lifestyle-related disease in the future) is high belongs, (ii) a category to which a subject whose future risk of lifestyle-related disease is low belongs, and (iii) a category to which a subject whose future risk of lifestyle-related disease is intermediate belongs. The categories may include (i) the category to which a subject whose future risk of lifestyle-related disease is high belongs and (ii) the category to which a subject whose future risk of lifestyle-related disease is low belongs.

If the future risk of lifestyle-related disease can be measured with successive numerical values, the value of the future risk of lifestyle-related disease in the subject may be estimated using the concentration value of the amino acid or using the concentration value of the amino acid and the formula including the explanatory variable to be substituted with the concentration value of the amino acid.

The concentration value or the value of the formula may be converted by a predetermined method, and the converted value may be used to classify the subject into any one of the plurality of the categories or estimate the value of the future risk of lifestyle-related disease in the subject.

The degree of the amount of insulin in the subject (for example, the amount of insulin in the subject's blood) may be qualitatively or quantitatively evaluated.

The subject may be classified into any one of a plurality of categories defined at least considering the degree of the amount of insulin, using “the concentration value of the amino acid and the one or more preset thresholds” or “the concentration value of the amino acid, the formula including the explanatory variable to be substituted with the concentration value of the amino acid, and the one or more preset thresholds”. The categories may include (i) a category to which a subject whose amount of insulin (for example, the 120-minute OGTT insulin level) is large belongs, (ii) a category to which a subject whose amount of insulin (for example, the 120-minute OGTT insulin level) is small belongs, and (iii) a category to which a subject whose amount of insulin (for example, the 120-minute OGTT insulin level) is medium belongs. The categories may include (i) a category to which a subject whose amount of insulin (for example, the 120-minute OGTT insulin level) is equal to or greater than a criterion value (for example, 40 μU/ml) belongs and (ii) a category to which a subject whose amount of insulin (for example, the 120-minute OGTT insulin level) is equal to or smaller than the criterion value (for example, 40 μU/ml) belongs. The categories may include (i) a category to which a subject with whom the possibility that the 120-minute OGTT insulin level is equal to or greater than 40 μU/ml is high belongs, (ii) a category to which a subject with whom the possibility is low belongs, and (iii) a category to which a subject with whom the possibility is intermediate belongs. The categories may include (i) the category to which a subject with whom the possibility that the 120-minute OGTT insulin level is equal to or greater than 40 μU/ml is high belongs and (ii) the category to which a subject with whom the possibility is low belongs.

The amount of insulin in the subject may be estimated using the concentration value of the amino acid and the formula including the explanatory variable to be substituted with the concentration value of the amino acid.

The concentration value or the value of the formula may be converted by a predetermined method, and the converted value may be used to classify the subject into any one of the plurality of the categories or estimate the amount of insulin in the subject.

The degree of the amount of visceral fat (for example, the value of the fat area in the axial section of the abdomen) in the subject may be evaluated.

The subject may be classified into any one of a plurality of categories defined at least considering the degree of the amount of visceral fat, using “the concentration value of the amino acid and the one or more preset thresholds” or “the concentration value of the amino acid, the formula including the explanatory variable to be substituted with the concentration value of the amino acid, and the one or more preset thresholds”. The categories may include (i) a category to which a subject who has a large amount of visceral fat (for example, a visceral fat area value) belongs, (ii) a category to which a subject who has a small amount of visceral fat (for example, a visceral fat area value) belongs, and (iii) a category to which a subject who has a medium amount of visceral fat (for example, a visceral fat area value) belongs. The categories may include (i) a category to which a subject whose amount of visceral fat (for example, a visceral fat area value) is equal to or greater than a criterion value (for example, 100 cm²) belongs and (ii) a category to which a subject whose amount of visceral fat (for example, a visceral fat area value) is equal to or smaller than the criterion value (for example, 100 cm²) belongs. The categories may include (i) a category to which a subject with whom the possibility that the visceral fat area value is equal to or greater than 100 cm² is high belongs, (ii) a category to which a subject with whom the possibility is low belongs, and (iii) a category to which a subject with whom the possibility is intermediate belongs. The categories may include (i) the category to which a subject with whom the possibility that the visceral fat area value is equal to or greater than 100 cm² is high belongs and (ii) the category to which a subject with whom the possibility is low belongs.

The amount of visceral fat in the subject may be estimated using the concentration value of the amino acid and the formula including the explanatory variable to be substituted with the concentration value of the amino acid.

The concentration value or the value of the formula may be converted by a predetermined method, and the converted value may be used to classify the subject into any one of the plurality of the categories or estimate the amount of visceral fat in the subject.

When the classification or the estimation is conducted, the BMI value of the subject and the formula further including the explanatory variable to be substituted with the BMI value may be used.

The degree of the possibility of fatty liver, that is, the degree of the possibility that the subject's liver is in a state of having a certain amount or more of fat (for example, the amount of fat exceeding 5% of the weight of the liver, the amount of fat equivalent to 30% or more of hepatocytes, or the amount of fat determined by doctors to be a fatty liver) may be evaluated.

The subject may be classified into any one of a plurality of categories defined at least considering the degree of the possibility that the liver is in the state above, using “the concentration value of the amino acid and the one or more preset thresholds” or “the concentration value of the amino acid, the formula including the explanatory variable to be substituted with the concentration value of the amino acid, and the one or more preset thresholds”. The categories may include (i) a category to which a subject with whom the possibility that the liver is in the state above is high belongs, (ii) a category to which a subject with whom the possibility that the liver is in the state above is low belongs, and (iii) a category to which a subject with whom the possibility that the liver is in the state above is intermediate belongs. The categories may include (i) the category to which a subject with whom the possibility that the liver is in the state above is high belongs and (ii) the category to which a subject with whom the possibility that the liver is in the state above is low belongs.

The concentration value or the value of the formula may be converted by a predetermined method, and the converted value may be used to classify the subject into any one of the plurality of the categories.

The formula may be any one of a logistic regression equation, a fractional expression, a linear discriminant, a multiple regression equation, a formula prepared by support vector machine, a formula prepared by Mahalanobis' generalized distance method, a formula prepared by canonical discriminant analysis, and a formula prepared by decision tree.

Among a plurality of items defined as diagnosis criteria items for metabolic syndrome, the number of items applicable to the subject may be evaluated using the concentration value of the amino acid and any one of a plurality of the formulae (for example, the formula used for evaluating the state of insulin, the formula used for evaluating the state of visceral fat, and the formula used for evaluating the state of fatty liver).

The number of lifestyle-related diseases that the subject has may be evaluated using the concentration value of the amino acid and any one of the plurality of the formulae (for example, the formula used for evaluating the state of insulin, the formula used for evaluating the state of visceral fat, and the formula used for evaluating the state of fatty liver).

The degree of the possibility that the subject is affected by lifestyle-related disease may be evaluated using the concentration value of the amino acid and any one of the plurality of the formulae (for example, the formula used for evaluating the state of insulin, the formula used for evaluating the state of visceral fat, and the formula used for evaluating the state of fatty liver).

In addition to the formulae described in the present specification, the formulae described in the international patent applications, filed by the present applicant, WO 2008/016111, WO 2008/075662, WO 2008/075663, WO 2009/099005, WO 2009/154296, and WO 2009/154297 can be additionally employed as the evaluation formulae to evaluate a state of the future risk of lifestyle-related disease.

The formula employed as the evaluation formula may be prepared by a method described in WO 2004/052191 that is an international application filed by the present applicant or by a method described in WO 2006/098192 that is an international application filed by the present applicant. Any formulae obtained by these methods can be preferably used in the evaluation of the state of the future risk of lifestyle-related disease, regardless of the unit of the amino acid concentration value in the amino acid concentration data as input data.

The formula employed as the evaluation formula refers to a form of equation used generally in multivariate analysis and includes, for example, the fractional expression, the multiple regression equation, the multiple logistic regression equation, the linear discriminant function, the Mahalanobis' generalized distance, the canonical discriminant function, the support vector machine, the decision tree, and an equation shown by the sum of different forms of equations. In the multiple regression equation, the multiple logistic regression equation, and the canonical discriminant function, a coefficient and a constant term are added to each explanatory variable, and the coefficient and the constant term may be preferably real numbers, more preferably values in the range of 99% confidence interval for the coefficient and the constant term obtained from data for the various kinds of classifications described above, more preferably in the range of 95% confidence interval for the coefficient and the constant term obtained from data for the various kinds of classifications described above. The value of each coefficient and the confidence interval thereof may be those multiplied by a real number, and the value of the constant term and the confidence interval thereof may be those having an arbitrary actual constant added or subtracted or those multiplied or divided by an arbitrary actual constant. When an expression such as the logistic regression, the linear discriminant, and the multiple regression equation is used as the evaluation formula, a linear transformation of the expression (addition of a constant and multiplication by a constant) and a monotonic increasing (decreasing) transformation (for example, a logit transformation) of the expression do not alter evaluation performance and thus are equivalent to before transformation. Therefore, the expression includes an expression that is subjected to a linear transformation and a monotonic increasing (decreasing) transformation.

In the fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, α+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In a fractional expression and the one in which explanatory variables in the numerator and explanatory variables in the denominator in the fractional expression are switched with each other, the positive and negative signs are generally reversed in correlation with objective explanatory variables, but because their correlation is maintained, the evaluation performance can be assumed to be equivalent. The fractional expression therefore also includes the one in which explanatory variables in the numerator and explanatory variables in the denominator in the fractional expression are switched with each other.

When the state of the future risk of lifestyle-related disease is evaluated, the concentration value of the amino acid other than the 21 kinds of amino acids above may be additionally used. When the state of the future risk of lifestyle-related disease is evaluated, the value related to other biological information (for example, values listed in 1. to 4. below) may further be used in addition to the concentration value of the amino acid. The formulae adopted as the evaluation formula may additionally include one or more explanatory variables to be substituted with the concentration values of amino acids other than the 21 kinds of amino acids. The formulae adopted as the evaluation formula may additionally include one or more explanatory variables to be substituted with the value related to other biological information (for example, values listed in 1. to 4. below) in addition to the explanatory variable to be substituted with the concentration value of the amino acid.

1. Concentration values of metabolites in blood other than amino acids (amino acid metabolites, carbohydrates, lipids, and the like), proteins, peptides, minerals, hormones, and the like.

2. Blood test values such as albumin, total protein, triglyceride, HbA1c, glycoalbumin, insulin resistance index, total cholesterol, LDL cholesterol, HDL cholesterol, amylase, total bilirubin, creatinine, estimated glomerular filtration rate (eGFR), uric acid.

3. Values obtained from image information such as ultrasonic echo, X ray, CT (Computer Tomography), and MRI (Magnetic Resonance Imaging).

4. Values of biological indices such as age, height, weight, BMI, abdominal girth, systolic blood pressure, diastolic blood pressure, gender, smoking information, dietary information, drinking information, exercise information, stress information, sleeping information, family medical history information, and disease history information (for example, diabetes).

When, before step S11 is executed, the desired substance group consisting of one or more substances is administered to the subject, and then blood is collected from the subject, and in step S11, the amino acid concentration data of the subject is obtained, a substance ameliorating the state of the future risk of lifestyle-related disease may be searched by judging whether or not the administered substance group ameliorates the state of the future risk of lifestyle-related disease, using the evaluation result obtained in step S12.

Before step S11 is executed, a suitable combination of an existing drug, amino acid, food and supplement capable of administration to humans (for example, a suitable combination of drugs known to be effective in amelioration of the future risk of lifestyle-related disease) may be administered over a predetermined period (for example in the range of 1 day to 12 months) in a predetermined amount at predetermined frequency and timing (for example 3 times per day, after food) by a predetermined administration method (for example, oral administration). The administration method, dose, and dosage form may be suitably combined depending on the condition of a patient. The dosage form may be determined based on known techniques. The dose is not particularly limited, and for example, a drug containing 1 μg to 100 g active ingredient may be given.

When the judgement result that the administered substance group ameliorates the state of the future risk of lifestyle-related disease is obtained, the administered substance group may be searched as a substance ameliorating the state of the future risk of lifestyle-related disease. The substance group searched by the searching method includes, for example, the amino acid group including the 21 kinds of amino acids.

Substances that restore normal values to the concentration values of the amino acid group including the 21 kinds of amino acids or the values of the evaluation formulae can be selected using the evaluating method in the first embodiment or the evaluating apparatus in the second embodiment.

Searching for a substance ameliorating the state of the future risk of lifestyle-related disease includes not only discovery of a novel substance effective in ameliorating the future risk of lifestyle-related disease, but also (i) new discovery of use of a known substance in ameliorating the future risk of lifestyle-related disease, (ii) discovery of a novel composition consisting of a combination of existing drugs, supplements etc. having efficacy expectable for amelioration of the future risk of lifestyle-related disease, (iii) discovery of the suitable usage, dose and combination described above to form them into a kit, (iv) presentation of a preventing and therapeutic menu including a diet, exercise etc., and (v) presentation of a necessary change in menu for each individual by monitoring the effect of the preventing and therapeutic menu.

### Second Embodiment

### 2-1. Outline of the Second Embodiment

Here, outlines of the second embodiment will be described in detail with reference to FIG. 2. FIG. 2 is a principle configurational diagram showing a basic principle of the second embodiment. In the description of the present second embodiment, description duplicating that of the first embodiment is sometimes omitted. In particular, herein, when the future risk of lifestyle-related disease is evaluated, a case of using the value of the evaluation formula or the converted value thereof is described as one example. However, for example, the concentration value of the amino acid or the converted value thereof (for example, the amino acid concentration standard score) may be used.

A control device evaluates the future risk of lifestyle-related disease for the subject by calculating the value of the formula using (i) the concentration value of the amino acid included in the previously obtained amino acid concentration data of the subject (for example, an individual such as animal or human) on the concentration value of the amino acid and (ii) the formula previously stored in a memory device including the explanatory variable to be substituted with the concentration value of the amino acid (step S21).

According to the second embodiment described above, in step S21, the future risk of lifestyle-related disease for the subject is evaluated by calculating the value of the evaluation formula using (i) the concentration value of the amino acid included in the amino acid concentration data of the subject and (ii) the formula stored in the memory device as the evaluation formula, including the explanatory variable to be substituted with the concentration value of the amino acid. Hence, reliable information that may be helpful in knowing the future risk of lifestyle-related disease can be provided.

Here, the summary of the evaluation formula-preparing processing (steps 1 to 4) is described in detail. The processing described below is merely one example, and the method of preparing the evaluation formula is not limited thereto.

First, the control device prepares a candidate formula (e.g., y=a₁x₁+a₂x₂+ . . . +aₙxₙ, y: lifestyle-related disease index data, xᵢ: amino acid concentration data, aᵢ: constant, i=1, 2, . . . , n) that is a candidate for the evaluation formula, based on a predetermined formula-preparing method from index state information previously stored in the memory device containing the amino acid concentration data and lifestyle-related disease index data on a state of an index of lifestyle-related disease (step 1). Data containing defective and outliers may be removed in advance from the index state information.

In step 1, a plurality of the candidate formulae may be prepared from the index state information by using a plurality of the different formula-preparing methods (including those for multivariate analysis such as principal component analysis, discriminant analysis, support vector machine, multiple regression analysis, logistic regression analysis, k-means method, cluster analysis, and decision tree). Specifically, a plurality of the candidate formulae may be prepared simultaneously and concurrently by using a plurality of different algorithms with the index state information which is multivariate data composed of the amino acid concentration data and the lifestyle-related disease index data obtained by analyzing blood obtained from a large number of healthy groups and groups having the index of lifestyle-related disease of being a predetermined state (for example, a state of exceeding a criterion value). For example, the two different candidate formulae may be formed by performing discriminant analysis and logistic regression analysis simultaneously with the different algorithms. Alternatively, the candidate formula may be formed by converting the index state information with the candidate formula prepared by performing principal component analysis and then performing discriminant analysis of the converted index state information. In this way, it is possible to finally prepare the most suitable evaluation formula.

The candidate formula prepared by principal component analysis is a linear expression including each amino acid explanatory variable maximizing the variance of all amino acid concentration data. The candidate formula prepared by discriminant analysis is a high-powered expression (including exponential and logarithmic expressions) including each amino acid explanatory variable minimizing the ratio of the sum of the variances in respective groups to the variance of all amino acid concentration data. The candidate formula prepared by using support vector machine is a high-powered expression (including kernel function) including each amino acid explanatory variable maximizing the boundary between groups. The candidate formula prepared by using multiple regression analysis is a high-powered expression including each amino acid explanatory variable minimizing the sum of the distances from all amino acid concentration data. The candidate formula prepared by using logistic regression analysis is a linear model expressing logarithmic odds of probability, and a linear expression including each amino acid explanatory variable maximizing the likelihood of the probability. The k-means method is a method of searching k pieces of neighboring amino acid concentration data in various groups, designating the group containing the greatest number of the neighboring points as its data-belonging group, and selecting the amino acid explanatory variable that makes the group to which input amino acid concentration data belong agree well with the designated group. The cluster analysis is a method of clustering (grouping) the points closest in entire amino acid concentration data. The decision tree is a method of ordering amino acid explanatory variables and predicting the group of amino acid concentration data from the pattern possibly held by the higher-ordered amino acid explanatory variable.

Returning to the description of the evaluation formula-preparing processing, the control device verifies (mutually verifies) the candidate formula prepared in step 1 based on a particular verifying method (step 2). The verification of the candidate formula is performed on each other to each candidate formula prepared in step 1.

In step 2, at least one of discrimination rate, sensitivity, specificity, information criterion, ROC_AUC (area under the curve in a receiver operating characteristic curve), and the like of the candidate formula may be verified by at least one of the bootstrap method, holdout method, N-fold method, leave-one-out method, and the like. In this way, it is possible to prepare the candidate formula higher in predictability or reliability, by taking the index state information and the evaluation condition into consideration.

The discrimination rate is a rate in which the negative state of the indicator of lifestyle-related disease evaluated as a true state in the present embodiment (for example, the result of definite diagnosis) is correctly evaluated as being negative and the positive state as a true state is correctly evaluated as being positive. The sensitivity refers to a rate in which the positive state of the indicator of lifestyle-related disease evaluated as a true state in the present embodiment is correctly evaluated as being positive. The specificity refers to a rate in which the negative state of the indicator of lifestyle-related disease evaluated as a true state in the present embodiment is correctly evaluated as being negative. The Akaike information criterion is a criterion representing how observation data agrees with a statistical model, for example, in regression analysis, and it is determined that the model in which the value defined by “−2×(maximum log-likelihood of statistical model)+2×(the number of free parameters of statistical model)” is smallest is the best. ROC_AUC (the area under the receiver operating characteristics curve) is defined as the area under the receiver operating characteristics curve (ROC) created by plotting (x, y)=(1-specificity, sensitivity) on two-dimensional coordinates. The value of ROC_AUC is 1 in perfect discrimination, and the closer this value is to 1, the higher the discriminative characteristic. The predictability is the average of discrimination rates, sensitivities, or specificities obtained by repeating the validation of the candidate formula. The robustness refers to the variance of discrimination rates, sensitivities, or specificities obtained by repeating the validation of the candidate formula.

Returning to the description of the evaluation formula-preparing processing, the control device selects a combination of the amino acid concentration data contained in the index state information used in preparing the candidate formula, by selecting an explanatory variable of the candidate formula based on a predetermined explanatory variable-selecting method (step 3). The selection of the amino acid explanatory variable may be performed on each candidate formula prepared in step 1. In this way, it is possible to select the amino acid explanatory variable of the candidate formula properly. The step 1 is executed once again by using the index state information including the amino acid concentration data selected in step 3.

In step 3, the amino acid explanatory variable of the candidate formula may be selected based on at least one of the stepwise method, best path method, local search method, and genetic algorithm from the verification result obtained in step 2.

The best path method is a method of selecting an amino acid explanatory variable by optimizing an evaluation index of the candidate formula while eliminating the amino acid explanatory variables contained in the candidate formula one by one.

Returning to the description of the evaluation formula-preparing processing, the control device prepares the evaluation formula by repeatedly performing the steps 1, 2 and 3, and based on verification results thus accumulated, selecting the candidate formula used as the evaluation formula from a plurality of the candidate formulae (step 4). In the selection of the candidate formula, there are cases where the optimum formula is selected from the candidate formulae prepared in the same formula-preparing method or the optimum formula is selected from all candidate formulae.

As described above, in the evaluation formula-preparing processing, the processing for the preparation of the candidate formulae, the verification of the candidate formulae, and the selection of the explanatory variables in the candidate formulae are performed based on the index state information in a series of operations in a systematized manner, whereby the evaluation formula most appropriate for evaluating the state of the indicator of lifestyle-related disease can be prepared. In other words, in the evaluation formula-preparing processing, the amino acid concentration is used in multivariate statistical analysis, and for selecting the optimum and robust combination of the explanatory variables, the explanatory variable-selecting method is combined with cross-validation to extract the evaluation formula having high evaluation performance. Logistic regression equation, linear discriminant, support vector machine, Mahalanobis' generalized distance method, multiple regression analysis, cluster analysis, Cox proportional-hazards model, and the like can be used as the evaluation formula.

### 2-2. System Configuration

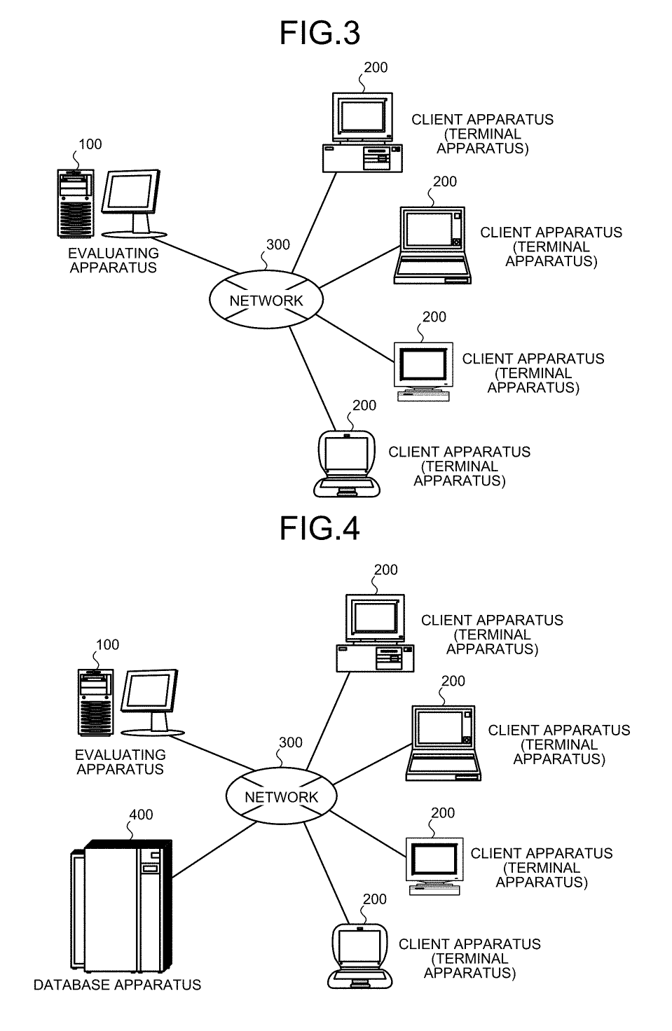

Hereinafter, the configuration of the evaluating system according to the second embodiment (hereinafter referred to sometimes as the present system) will be described with reference to FIGS. 3 to 18. This system is merely one example, and the present invention is not limited thereto. In particular, herein, when the future risk of lifestyle-related disease is evaluated, a case of using the value of the evaluation formula or the converted value thereof is described as one example. However, for example, the concentration value of the amino acid or the converted value thereof (for example, the amino acid concentration standard score) may be used.

First, an entire configuration of the present system will be described with reference to FIGS. 3 and 4. FIG. 3 is a diagram showing an example of the entire configuration of the present system. FIG. 4 is a diagram showing another example of the entire configuration of the present system. As shown in FIG. 3, the present system is constituted in which the evaluating apparatus 100 that evaluates the future risk of lifestyle-related disease in the individual as the subject, and the client apparatus 200 (corresponding to the terminal apparatus of the present invention) that provides the amino acid concentration data of the individual on the concentration value of the amino acid, are communicatively connected to each other via a network 300.

In the present system as shown in FIG. 4, in addition to the evaluating apparatus 100 and the client apparatus 200, the database apparatus 400 storing, for example, the index state information used in preparing the evaluation formula and the evaluation formula used in evaluating the future risk of lifestyle-related disease in the evaluating apparatus 100, may be communicatively connected via the network 300. In this configuration, reliable information that may be helpful in knowing the future risk of lifestyle-related disease or the like is provided via the network 300 from the evaluating apparatus 100 to the client apparatuses 200 and the database apparatus 400, or from the client apparatuses 200 and the database apparatus 400 to the evaluating apparatus 100. The reliable information that may be helpful in knowing the future risk of lifestyle-related disease is, for example, information on the measured value of a particular item as to the future risk of lifestyle-related disease of organisms including human. The reliable information that may be helpful in knowing the future risk of lifestyle-related disease is generated in the evaluating apparatus 100, client apparatus 200, or other apparatuses (e.g., various measuring apparatuses) and stored mainly in the database apparatus 400.

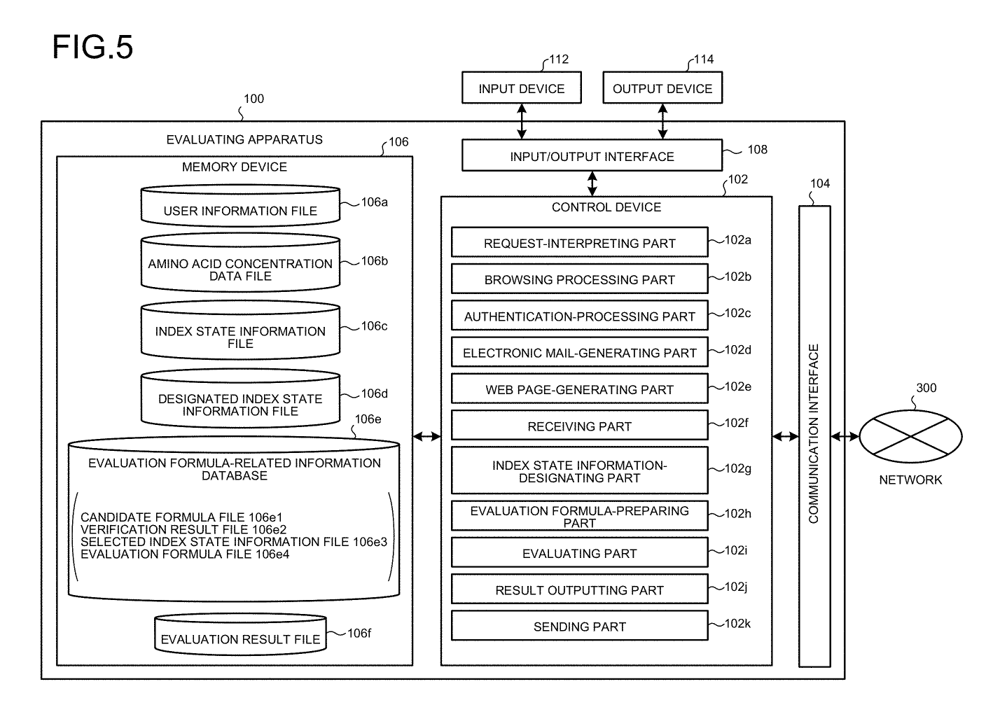

Now, the configuration of the evaluating apparatus 100 in the present system will be described with reference to FIGS. 5 to 16. FIG. 5 is a block diagram showing an example of the configuration of the evaluating apparatus 100 in the present system, showing conceptually only the region relevant to the present invention.

The evaluating apparatus 100 includes (I) a control device 102, such as CPU (Central Processing Unit), that integrally controls the evaluating apparatus, (II) a communication interface 104 that connects the evaluating apparatus to the network 300 communicatively via communication apparatuses such as a router and wired or wireless communication lines such as a private line, (III) a memory device 106 that stores various databases, tables, files and others, and (IV) an input/output interface 108 connected to an input device 112 and an output device 114, and these parts are connected to each other communicatively via any communication channel. The evaluating apparatus 100 may be present together with various analyzers (e.g., amino acid analyzer) in a same housing. For example, the evaluating apparatus 100 may be a compact analyzing device including components (hardware and software) that calculate (measure) the concentration value of an amino acid in blood and output (e.g., print or display on a monitor) the calculated concentration value, wherein the compact analyzing device is characterized by further including the evaluating part 102i described later, and using the components to output results obtained by the evaluating part 102i.

The memory device 106 is a storage means, and examples thereof include memory apparatuses such as RAM (Random Access Memory) and ROM (Read Only Memory), fixed disk drives such as a hard disk, a flexible disk, an optical disk, and the like. The memory device 106 stores computer programs giving instructions to the CPU for various processings, together with OS (Operating System). As shown in the figure, the memory device 106 stores the user information file 106a, the amino acid concentration data file 106b, the index state information file 106c, the designated index state information file 106d, an evaluation formula-related information database 106e, and the evaluation result file 106f.

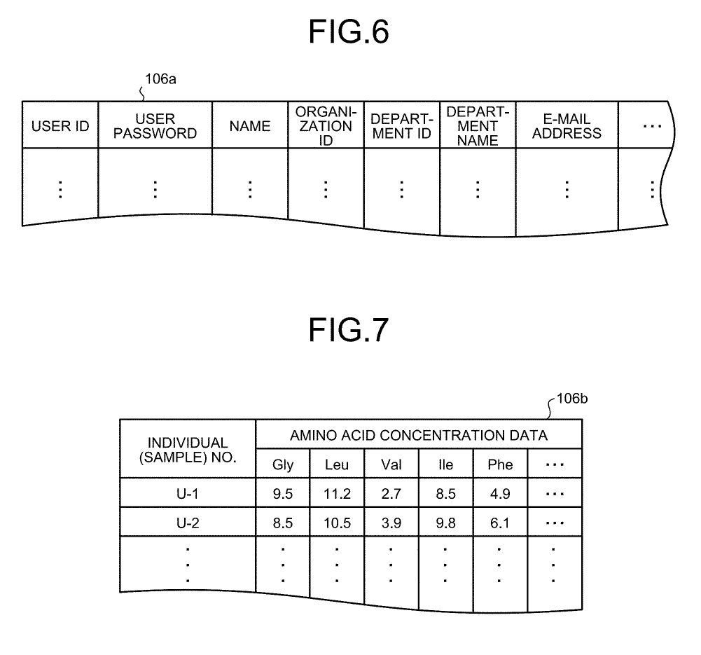

The user information file 106a stores user information on users. FIG. 6 is a chart showing an example of information stored in the user information file 106a. As shown in FIG. 6, the information stored in the user information file 106a includes a user ID (identification) for identifying a user uniquely, a user password for authentication of the user, a user name, an organization ID for uniquely identifying an organization of the user, a department ID for uniquely identifying a department of the user organization, a department name, and an electronic mail address of the user that are correlated to one another.

Returning to FIG. 5, the amino acid concentration data file 106b stores the amino acid concentration data on the concentration value of the amino acid. FIG. 7 is a chart showing an example of information stored in the amino acid concentration data file 106b. As shown in FIG. 7, the information stored in the amino acid concentration data file 106b includes an individual number for uniquely identifying the individual (sample) as the subject and the amino acid concentration data that are correlated to one another. In FIG. 7, the amino acid concentration data is assumed to be numerical values, i.e., on a continuous scale, but the amino acid concentration data may be expressed on a nominal scale or an ordinal scale. In the case of the nominal or ordinal scale, any number may be allocated to each state for analysis. The amino acid concentration data may be combined with the concentration value of the amino acid other than the 21 kinds of amino acids or the value of other biological information (for example, values listed in 1. to 4. below).

1. Concentration values of metabolites in blood other than amino acids (amino acid metabolites, carbohydrates, lipids, and the like), proteins, peptides, minerals, hormones, and the like.

2. Blood test values such as albumin, total protein, triglyceride, HbA1c, glycoalbumin, insulin resistance index, total cholesterol, LDL cholesterol, HDL cholesterol, amylase, total bilirubin, creatinine, estimated glomerular filtration rate (eGFR), uric acid.

3. Values obtained from image information such as ultrasonic echo, X ray, CT, and MRI.

4. Values of biological indices such as age, height, weight, BMI, abdominal girth, systolic blood pressure, diastolic blood pressure, gender, smoking information, dietary information, drinking information, exercise information, stress information, sleeping information, family medical history information, and disease history information (for example, diabetes).

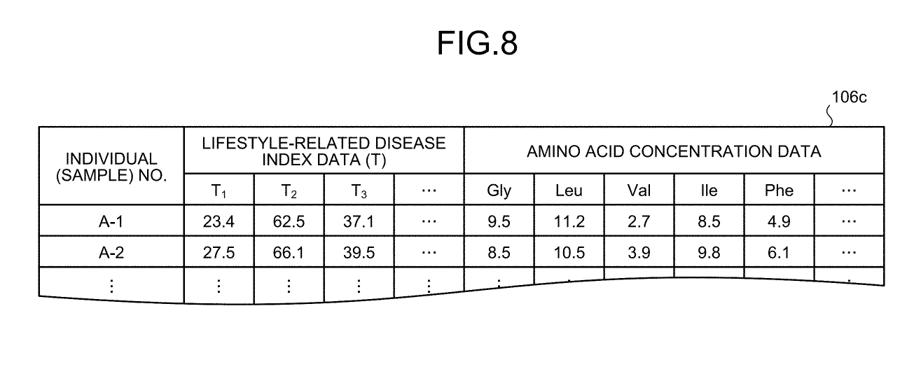

Returning to FIG. 5, the index state information file 106c stores the index state information used in preparing the evaluation formula. FIG. 8 is a chart showing an example of information stored in the index state information file 106c. As shown in FIG. 8, the information stored in the index state information file 106c includes the individual (sample) number, the lifestyle-related disease index data (T) on the state of an index (index T₁, index T₂, index T₃ . . . ) of lifestyle-related disease, and the amino acid concentration data that are correlated to one another. In FIG. 8, the lifestyle-related disease index data and the amino acid concentration data are assumed to be numerical values, i.e., on a continuous scale, but the lifestyle-related disease index data and the amino acid concentration data may be expressed on a nominal scale or an ordinal scale. In the case of the nominal or ordinal scale, any number may be allocated to each state for analysis. The lifestyle-related disease index data is a known index serving as a marker of lifestyle-related disease, and so on, and numerical data may be used.

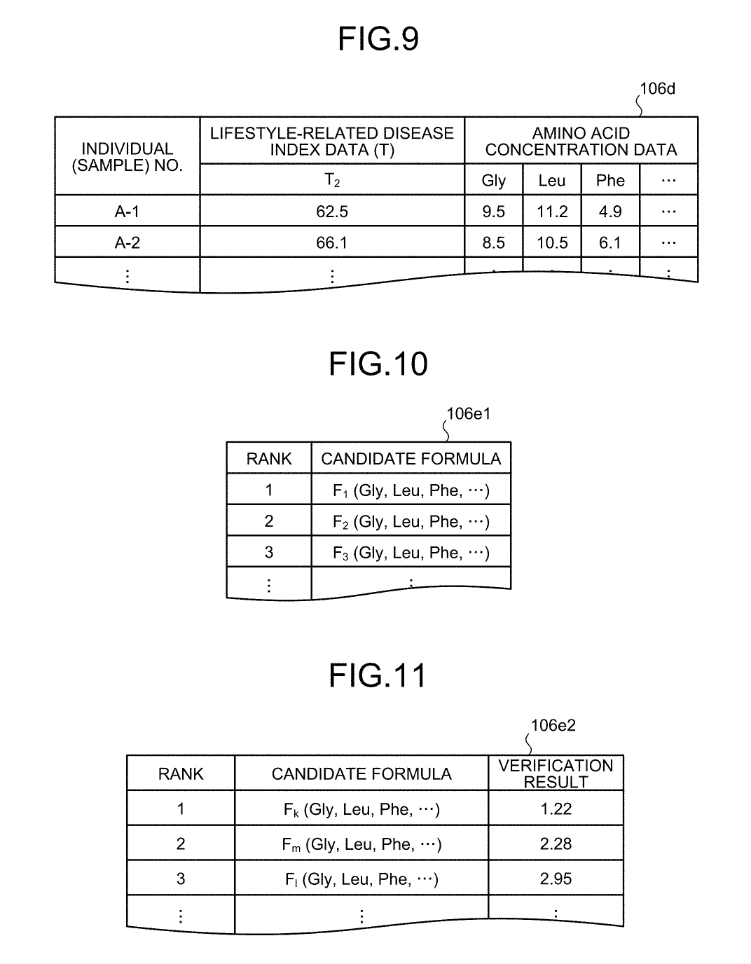

Returning to FIG. 5, the designated index state information file 106d stores the index state information designated in an index state information-designating part 102g described below. FIG. 9 is a chart showing an example of information stored in the designated index state information file 106d. As shown in FIG. 9, the information stored in the designated index state information file 106d includes the individual number, the designated lifestyle-related disease index data, and the designated amino acid concentration data that are correlated to one another.

Returning to FIG. 5, the evaluation formula-related information database 106e is composed of (I) the candidate formula file 106e1 storing the candidate formula prepared in a candidate formula-preparing part 102h1 described below, (II) the verification result file 106e2 storing the verification results obtained in a candidate formula-verifying part 102h2 described below, (III) the selected index state information file 106e3 storing the index state information containing the combination of the amino acid concentration data selected in an explanatory variable-selecting part 102h3 described below, and (IV) the evaluation formula file 106e4 storing the evaluation formula prepared in the evaluation formula-preparing part 102h described below.

The candidate formula file 106e1 stores the candidate formulae prepared in the candidate formula-preparing part 102h1 described below. FIG. 10 is a chart showing an example of information stored in the candidate formula file 106e1. As shown in FIG. 10, the information stored in the candidate formula file 106e1 includes a rank, and the candidate formula (e.g., F₁ (Gly, Leu, Phe, . . . ), F₂ (Gly, Leu, Phe, . . . ), or F₃ (Gly, Leu, Phe, . . . ) in FIG. 10) that are correlated to each other.

Returning to FIG. 5, the verification result file 106e2 stores the verification results obtained in the candidate formula-verifying part 102h2 described below. FIG. 11 is a chart showing an example of information stored in the verification result file 106e2. As shown in FIG. 11, the information stored in the verification result file 106e2 includes the rank, the candidate formula (e.g., Fₖ (Gly, Leu, Phe, . . . ), Fₘ (Gly, Leu, Phe, . . . ), F₁ (Gly, Leu, Phe, . . . ) in FIG. 11), and the verification result of each candidate formula (e.g., the evaluation value of each candidate formula) that are correlated to one another.

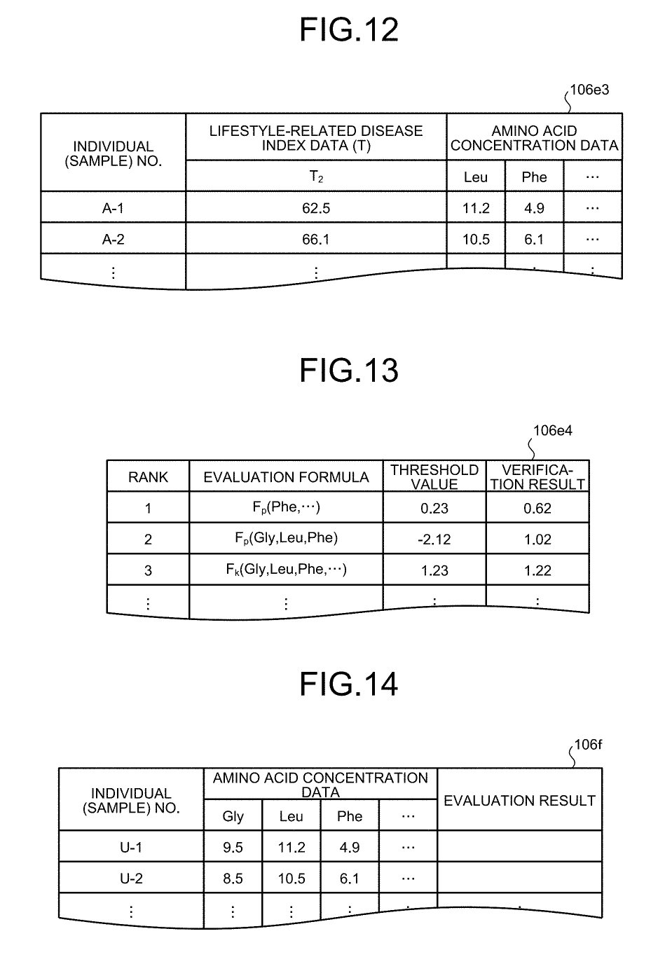

Returning to FIG. 5, the selected index state information file 106e3 stores the index state information including the combination of the amino acid concentration data corresponding to the explanatory variables selected in the explanatory variable-selecting part 102h3 described below. FIG. 12 is a chart showing an example of information stored in the selected index state information file 106e3. As shown in FIG. 12, the information stored in the selected index state information file 106e3 includes the individual number, the lifestyle-related disease index data designated in the index state information-designating part 102g described below, and the amino acid concentration data selected in the explanatory variable-selecting part 102h3 described below that are correlated to one another.

Returning to FIG. 5, the evaluation formula file 106e4 stores the evaluation formulae prepared in the evaluation formula-preparing part 102h described below. FIG. 13 is a chart showing an example of information stored in the evaluation formula file 106e4. As shown in FIG. 13, the information stored in the evaluation formula file 106e4 includes the rank, the evaluation formula (e.g., Fₚ (Phe, . . . ), Fₚ (Gly, Leu, Phe), Fₖ (Gly, Leu, Phe, . . . ) in FIG. 13), the threshold corresponding to each formula-preparing method, and the verification result of each evaluation formula (e.g., the evaluation value of each evaluation formula) that are correlated to one another.

Returning to FIG. 5, the evaluation result file 106f stores the evaluation results obtained in the evaluating part 102i described below. FIG. 14 is a chart showing an example of information stored in the evaluation result file 106f. The information stored in the evaluation result file 106f includes the individual number for uniquely identifying the individual (sample) as the subject, the previously obtained amino acid concentration data of the individual, and the evaluation result on the state of the indicator of lifestyle-related disease (for example, the value of the evaluation formula calculated by a calculating part 102i1 described below, the converted value of the evaluation formula by a converting part 102i2 described below, the positional information generated by a generating part 102i3 described below, or the classification result obtained by a classifying part 102i4 described below), that are correlated to one another.

Returning to FIG. 5, the memory device 106 stores various Web data for providing the client apparatuses 200 with web site information, CGI programs, and others as information other than the information described above. The Web data include data for displaying the Web pages described below and others, and the data are generated as, for example, a HTML (HyperText Markup Language) or XML (Extensible Markup Language) text file. Files for components and files for operation for generation of the Web data, and other temporary files, and the like are also stored in the memory device 106. In addition, the memory device 106 may store as needed sound files of sounds for transmission to the client apparatuses 200 in WAVE format or AIFF (Audio Interchange File Format) format and image files of still images or motion pictures in JPEG (Joint Photographic Experts Group) format or MPEG2 (Moving Picture Experts Group phase 2) format.

The communication interface 104 allows communication between the evaluating apparatus 100 and the network 300 (or communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as a monitor 114). A keyboard, a mouse, a microphone, or a monitor functioning as a pointing device together with a mouse may be used as the input device 112.

The control device 102 has an internal memory storing control programs such as OS (Operating System), programs for various processing procedures, and other needed data, and performs various information processings according to these programs. As shown in the figure, the control device 102 includes mainly a request-interpreting part 102a, a browsing processing part 102b, an authentication-processing part 102c, an electronic mail-generating part 102d, a Web page-generating part 102e, a receiving part 102f, the index state information-designating part 102g, the evaluation formula-preparing part 102h, the evaluating part 102i, a result outputting part 102j and a sending part 102k. The control device 102 performs data processings such as removal of data including defective, removal of data including many outliers, and removal of explanatory variables for the defective-including data in the index state information transmitted from the database apparatus 400 and in the amino acid concentration data transmitted from the client apparatus 200.

The request-interpreting part 102a interprets the requests transmitted from the client apparatus 200 or the database apparatus 400 and sends the requests to other parts in the control device 102 according to results of interpreting the requests. Upon receiving browsing requests for various screens transmitted from the client apparatus 200, the browsing processing part 102b generates and transmits web data for these screens. Upon receiving authentication requests transmitted from the client apparatus 200 or the database apparatus 400, the authentication-processing part 102c performs authentication. The electronic mail-generating part 102d generates electronic mails including various kinds of information. The Web page-generating part 102e generates Web pages for users to browse with the client apparatus 200.

The receiving part 102f receives, via the network 300, information (specifically, the amino acid concentration data, the index state information, the evaluation formula, etc.) transmitted from the client apparatus 200 and the database apparatus 400. The index state information-designating part 102g designates objective lifestyle-related disease index data and objective amino acid concentration data in preparing the evaluation formula.

The evaluation formula-preparing part 102h generates the evaluation formula based on the index state information received in the receiving part 102f and the index state information designated in the index state information-designating part 102g. Specifically, the evaluation formula-preparing part 102h generates the evaluation formula by selecting the candidate formula used as the evaluation formula from the plurality of the candidate formulae, based on the verification results accumulated by repeating processings in the candidate formula-preparing part 102h1, the candidate formula-verifying part 102h2, and the explanatory variable-selecting part 102h3 from the index state information.

If the evaluation formulae are stored previously in a predetermined region of the memory device 106, the evaluation formula-preparing part 102h may generate the evaluation formula by selecting the desired evaluation formula out of the memory device 106. Alternatively, the evaluation formula-preparing part 102h may generate the evaluation formula by selecting and downloading the desired evaluation formula from the evaluation formulae previously stored in another computer apparatus (e.g., the database apparatus 400).

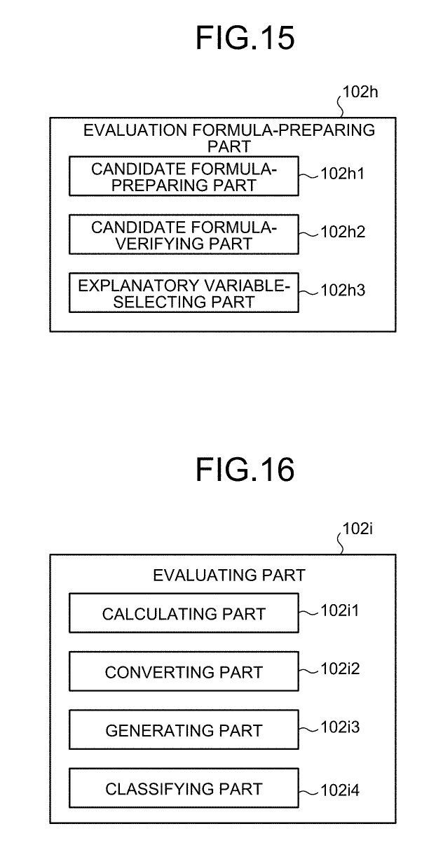

Hereinafter, a configuration of the evaluation formula-preparing part 102h will be described with reference to FIG. 15. FIG. 15 is a block diagram showing the configuration of the evaluation formula-preparing part 102h, and only a part in the configuration related to the present invention is shown conceptually. The evaluation formula-preparing part 102h includes the candidate formula-preparing part 102h1, the candidate formula-verifying part 102h2, and the explanatory variable-selecting part 102h3, additionally. The candidate formula-preparing part 102h1 generates the candidate formula that is the candidate of the evaluation formula, from the index state information based on the predetermined formula-preparing method. The candidate formula-preparing part 102h1 may generate the plurality of the candidate formulae from the index state information, by using the plurality of the different formula-preparing methods. The candidate formula-verifying part 102h2 verifies the candidate formula prepared by the candidate formula-preparing part 102h1 based on the particular verifying method. The candidate formula-verifying part 102h2 may verify at least one of the discrimination rate, sensitivity, specificity, information criterion, and ROC_AUC (area under the curve in a receiver operating characteristic curve) of the candidate formulae based on at least one of the bootstrap method, holdout method, N-fold method, and leave-one-out method. The explanatory variable-selecting part 102h3 selects the combination of the amino acid concentration data contained in the index state information used in preparing the candidate formula, by selecting the explanatory variables of the candidate formula based on the particular explanatory variable-selecting method. The explanatory variable-selecting part 102h3 may select the explanatory variables of the candidate formula based on at least one of the stepwise method, best path method, local search method, and genetic algorithm from the verification results.

Returning to FIG. 5, the evaluating part 102i evaluates the future risk of lifestyle-related disease for the individual by calculating the value of the evaluation formula using the previously obtained formula (for example, the evaluation formula prepared by the evaluation formula-preparing part 102h or the evaluation formula received by the receiving part 102f) and the amino acid concentration data received by the receiving part 102f. The evaluating part 102i may evaluate the future risk of lifestyle-related disease for the individual using the concentration value of the amino acid or the converted value of the concentration value (for example, the amino acid concentration standard score).

Hereinafter, a configuration of the evaluating part 102i will be described with reference to FIG. 16. FIG. 16 is a block diagram showing the configuration of the evaluating part 102i, and only a part in the configuration related to the present invention is shown conceptually. The evaluating part 102i includes the calculating part 102i1, the converting part 102i2, the generating part 102i3, and the classifying part 102i4, additionally.

The calculating part 102i1 calculates the value of the evaluation formula using the concentration value of the amino acid and the evaluation formula including the explanatory variable to be substituted with the concentration value of the amino acid. The evaluating part 102i may store the value of the evaluation formula calculated by the calculating part 102i1 as the evaluation result in a predetermined region of the evaluation result file 106f. The evaluation formula may be any one of the logistic regression equation, the fractional expression, the linear discriminant, the multiple regression equation, the formula prepared by support vector machine, the formula prepared by Mahalanobis' generalized distance method, the formula prepared by canonical discriminant analysis, and the formula prepared by decision tree. If the future risk of lifestyle-related disease can be measured with successive numerical values, the evaluating part 102i may regard the value of the evaluation formula calculated by the calculating part 102i1 as the estimation value of the future risk of lifestyle-related disease.

The converting part 102i2 converts the value of the evaluation formula calculated by the calculating part 102i1, for example, by the conversion method described above. The evaluating part 102i may store the converted value by the converting part 102i2 as the evaluation result in a predetermined region of the evaluation result file 106f. If the future risk of lifestyle-related disease can be measured with successive numerical values, the evaluating part 102i may regard the converted value by the converting part 102i2 as the estimation value of the future risk of lifestyle-related disease. The converting part 102i2 may convert the concentration value of the amino acids included in the amino acid concentration data, for example, by the conversion method described above. For example, the converting part 102i2 may convert the concentration value of the amino acid into the amino acid concentration standard score (standardization).

The generating part 102i3 generates the positional information about the position of the predetermined mark (for example, a circle sign or a star sign) corresponding to the value of the formula or the converted value (the concentration value or the converted value of the concentration value may be used as well) on the predetermined scale (for example, a graduated scale at least marked with graduations corresponding to the upper limit value and the lower limit value in the possible range of the value of the formula or the converted value (the concentration value or the converted value of the concentration value may be used as well), or part of the range) visually presented on the display device such as the monitor or the physical medium such as paper for evaluating the future risk of lifestyle-related disease, using the value of the formula calculated by the calculating part 102i1 or the converted value by the converting part 102i2 (the concentration value or the converted value of the concentration value may be used as well). The evaluating part 102i may store the positional information generated by the generating part 102i3 as the evaluation result in a predetermined region of the evaluation result file 106f.

The classifying part 102i4 classifies the individual into any one of the plurality of the categories defined at least considering the degree of the future risk of lifestyle-related disease, using the value of the evaluation formula calculated by the calculating part 102i1 or the converted value by the converting part 102i2 (the concentration value or the converted value of the concentration value may be used as well).

Returning to FIG. 5, the result outputting part 102j outputs, into the output device 114, the processing results in each processing part in the control device 102 (including the evaluation results obtained by the evaluating part 102i) etc.

The sending part 102k transmits the evaluation results to the client apparatus 200 that is a sender of the amino acid concentration data of the individual, and transmits the evaluation formulae prepared in the evaluating apparatus 100 and the evaluation results to the database apparatus 400.

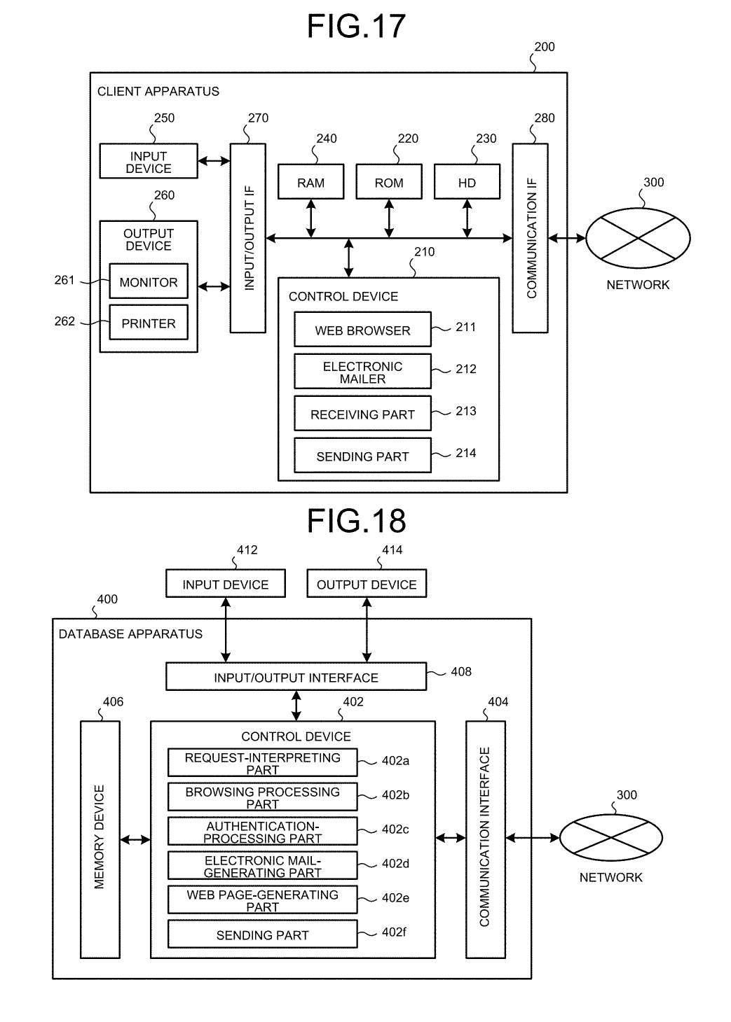

Hereinafter, a configuration of the client apparatus 200 in the present system will be described with reference to FIG. 17. FIG. 17 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel.

The control device 210 has a Web browser 211, an electronic mailer 212, a receiving part 213, and a sending part 214. The Web browser 211 performs browsing processings of interpreting Web data and displaying the interpreted Web data on a monitor 261 described below. The Web browser 211 may have various plug-in softwares, such as stream player, having functions to receive, display and feedback streaming screen images. The electronic mailer 212 sends and receives electronic mails using a particular protocol (e.g., SMTP (Simple Mail Transfer Protocol) or POPS (Post Office Protocol version 3)). The receiving part 213 receives various kinds of information, such as the evaluation results transmitted from the evaluating apparatus 100, via the communication IF 280. The sending part 214 sends various kinds of information such as the amino acid concentration data of the individual, via the communication IF 280, to the evaluating apparatus 100.

The input device 250 is for example a keyboard, a mouse or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatuses 200 are connected to the network 300 via a communication apparatus such as a modem, TA (Terminal Adapter) or a router, and a telephone line, or a private line. In this way, the client apparatuses 200 can access to the evaluating apparatus 100 by using a particular protocol.

The client apparatus 200 may be realized by installing softwares (including programs, data and others) for a Web data-browsing function and an electronic mail-processing function to an information processing apparatus (for example, an information processing terminal such as a known personal computer, a workstation, a family computer, Internet TV (Television), PHS (Personal Handyphone System) terminal, a mobile phone terminal, a mobile unit communication terminal or PDA (Personal Digital Assistants)) connected as needed with peripheral devices such as a printer, a monitor, and an image scanner.

All or a part of processings of the control device 210 in the client apparatus 200 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

The control device 210 may include an evaluating part 210a (including a calculating part 210a1, a converting part 210a2, a generating part 210a3, and a classifying part 210a4) having the same functions as the functions of the evaluating part 102i in the control device 102 of the evaluating apparatus 100. When the control device 210 includes the evaluating part 210a, the evaluating part 210a may convert the value of the formula in the converting part 210a2, generate the positional information corresponding to the value of the formula or the converted value (the concentration value or the converted value of the concentration value may be used as well) in the generating part 210a3, and classify the individual into any one of the categories using the value of the formula or the converted value (the concentration value or the converted value of the concentration value may be used as well) in the classifying part 210a4, in accordance with information included in the evaluation result transmitted from the evaluating apparatus 100.

Hereinafter, the network 300 in the present system will be described with reference to FIGS. 3 and 4. The network 300 has a function to connect the evaluating apparatus 100, the client apparatuses 200, and the database apparatus 400 mutually, communicatively to one another, and is for example the Internet, an intranet, or LAN (Local Area Network (including both wired and wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (registered trademark) (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

Hereinafter, the configuration of the database apparatus 400 in the present system will be described with reference to FIG. 18. FIG. 18 is a block diagram showing an example of the configuration of the database apparatus 400 in the present system, showing conceptually only the region relevant to the present invention.

The database apparatus 400 has functions to store, for example, (i) the index state information used in preparing the evaluation formulae in the evaluating apparatus 100 or in the database apparatus, (ii) the evaluation formulae prepared in the evaluating apparatus 100, and (iii) the evaluation results obtained in the evaluating apparatus 100. As shown in FIG. 18, the database apparatus 400 includes (I) a control device 402, such as CPU, which integrally controls the entire database apparatus, (II) a communication interface 404 connecting the database apparatus to the network 300 communicatively via a communication apparatus such as a router and via wired or wireless communication circuits such as a private line, (III) a memory device 406 storing various databases, tables and files (for example, files for Web pages), and (IV) an input/output interface 408 connected to an input device 412 and an output device 414, and these parts are connected communicatively to each other via any communication channel.

The memory device 406 is a storage means, and may be, for example, memory apparatus such as RAM or ROM, a fixed disk drive such as a hard disk, a flexible disk, an optical disk, and the like. The memory device 406 stores, for example, various programs used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414 (hereinafter, the output device 414 may be described as a monitor 414). A keyboard, a mouse, a microphone, or a monitor functioning as a pointing device together with a mouse may be used as the input device 412.

The control device 402 has an internal memory storing control programs such as OS (Operating System), programs for various processing procedures, and other needed data, and performs various information processings according to these programs. As shown in the figure, the control device 402 includes mainly a request-interpreting part 402a, a browsing processing part 402b, an authentication-processing part 402c, an electronic mail-generating part 402d, a Web page-generating part 402e, and a sending part 402f.

The request-interpreting part 402a interprets the requests transmitted from the evaluating apparatus 100 and sends the requests to other parts in the control device 402 according to results of interpreting the requests. Upon receiving browsing requests for various screens transmitted from the evaluating apparatus 100, the browsing processing part 402b generates and transmits web data for these screens. Upon receiving authentication requests transmitted from the evaluating apparatus 100, the authentication-processing part 402c performs authentication. The electronic mail-generating part 402d generates electronic mails including various kinds of information. The Web page-generating part 402e generates Web pages for users to browse with the client apparatus 200. The sending part 402f transmits various kinds of information such as the index state information and the evaluation formulae to the evaluating apparatus 100.

### 2-3. Specific Example of the Second Embodiment

Here, a specific example of the second embodiment will be described.

First, the client apparatus 200 accesses the evaluating apparatus 100 when the user specifies the Web site address (such as URL) provided from the evaluating apparatus 100, via the input device 250 on the screen displaying the Web browser 211. Specifically, when the user instructs update of the Web browser 211 screen on the client apparatus 200, the Web browser 211 sends the Web site address provided from the evaluating apparatus 100 by a particular protocol to the evaluating apparatus 100, thereby transmitting requests demanding a transmission of Web page corresponding to an amino acid concentration data transmission screen to the evaluating apparatus 100 based on a routing of the address.

Then, upon receipt of the request transmitted from the client apparatus 200, the request-interpreting part 102a in the evaluating apparatus 100 analyzes the transmitted requests and sends the requests to other parts in the control device 102 according to analytical results. Specifically, when the transmitted requests are requests to send the Web page corresponding to the amino acid concentration data transmission screen, mainly the browsing processing part 102b in the evaluating apparatus 100 obtains the Web data for display of the Web page stored in a predetermined region of the memory device 106 and sends the obtained Web data to the client apparatus 200. More specifically, upon receiving the requests to transmit the Web page corresponding to the amino acid concentration data transmission screen by the user, the control device 102 in the evaluating apparatus 100 demands inputs of user ID and user password from the user. If the user ID and password are input, the authentication-processing part 102c in the evaluating apparatus 100 examines the input user ID and password by comparing them with the user ID and user password stored in the user information file 106a for authentication. Only when the user is authenticated, the browsing processing part 102b in the evaluating apparatus 100 sends the Web data for displaying the Web page corresponding to the amino acid concentration data transmission screen to the client apparatus 200. The client apparatus 200 is identified with the IP (Internet Protocol) address transmitted from the client apparatus 200 together with the transmission requests.

Then, the client apparatus 200 receives, in the receiving part 213, the Web data (for displaying the Web page corresponding to the amino acid concentration data transmission screen) transmitted from the evaluating apparatus 100, interprets the received Web data with the Web browser 211, and displays the amino acid concentration data transmission screen on the monitor 261.

When the user inputs and selects, via the input device 250, for example the amino acid concentration data of the individual on the amino acid concentration data transmission screen displayed on the monitor 261, the sending part 214 of the client apparatus 200 transmits an identifier for identifying input information and selected items to the evaluating apparatus 100, thereby transmitting the amino acid concentration data of the individual to the evaluating apparatus 100 (step SA21). In step SA21, the transmission of the amino acid concentration data may be realized for example by using an existing file transfer technology such as FTP (File Transfer Protocol).

Then, the request-interpreting part 102a of the evaluating apparatus 100 interprets the identifier transmitted from the client apparatus 200 thereby interpreting the requests from the client apparatus 200, and requests the database apparatus 400 to send the evaluation formula.

Then, the request-interpreting part 402a in the database apparatus 400 interprets the transmission requests from the evaluating apparatus 100 and transmits, to the evaluating apparatus 100, the evaluation formula (for example, the updated newest evaluation formula) stored in a predetermined region of the memory device 406 (step SA22). Specifically, in step SA22, one or more evaluation formulae (for example, any one of the logistic regression equation, the fractional expression, the linear discriminant, the multiple regression equation, the formula prepared by support vector machine, the formula prepared by Mahalanobis' generalized distance method, the formula prepared by canonical discriminant analysis, and the formula prepared by decision tree) are transmitted to the evaluating apparatus 100.

Then, the evaluating apparatus 100 receives, in the receiving part 102f, the amino acid concentration data of the individual transmitted from the client apparatuses 200 and the evaluation formula transmitted from the database apparatus 400, and stores the received amino acid concentration data in a predetermined memory region of the amino acid concentration data file 106b and the received evaluation formula in a predetermined memory region of the evaluation formula file 106e4 (step SA23).

Then, the control device 102 in the evaluating apparatus 100 removes data such as defective and outliers from the amino acid concentration data of the individual received in step SA23 (step SA24).

The evaluating part 102i then uses the amino acid concentration data of the individual from which the data such as defective and outliers has been removed in step SA24 and the evaluation formula received in step SA23 to calculate the value of the evaluation formula in the calculating part 102i1 (step SA25).

The evaluating part 102i estimates the future risk of lifestyle-related disease for the indivisual using the value of evaluation formula calculated in step SA25. The evaluating part 102i classifies the individual into any one of the plurality of the categories defined at least considering the degree of the future risk of lifestyle-related disease, using the value of the evaluation formula (the evaluation value) calculated in step SA25 and the preset thethold in the classifying part 102i4. The evaluating part 102i stores the evaluation results including the obtained estimation result and the classification result in a predetermined memory region of the evaluation result file 106f (step SA26).

The sending part 102k in the evaluating apparatus 100 sends, to the client apparatus 200 that has sent the amino acid concentration data and to the database apparatus 400, the evaluation results obtained in step SA26 (step SA27). Specifically, the evaluating apparatus 100 first generates a Web page for displaying the evaluation results in the Web page-generating part 102e and stores the Web data corresponding to the generated Web page in a predetermined memory region of the memory device 106. Then, the user is authenticated as described above by inputting a predetermined URL (Uniform Resource Locator) into the Web browser 211 of the client apparatus 200 via the input device 250, and the client apparatus 200 sends a Web page browsing request to the evaluating apparatus 100. The evaluating apparatus 100 then interprets the browsing request transmitted from the client apparatus 200 in the browsing processing part 102b and reads the Web data corresponding to the Web page for displaying the evaluation results, out of the predetermined memory region of the memory device 106. The sending part 102k in the evaluating apparatus 100 then sends the read-out Web data to the client apparatus 200 and simultaneously sends the Web data or the evaluation results to the database apparatus 400.

In step SA27, the control device 102 in the evaluating apparatus 100 may notify the evaluation results to the user client apparatus 200 by electronic mail. Specifically, the electronic mail-generating part 102d in the evaluating apparatus 100 first acquires the user electronic mail address by referencing the user information stored in the user information file 106a based on the user ID and the like at the transmission timing. The electronic mail-generating part 102d in the evaluating apparatus 100 then generates electronic mail data with the acquired electronic mail address as its mail address, including the user name and the evaluation results. The sending part 102k in the evaluating apparatus 100 then sends the generated electronic mail data to the user client apparatus 200.

Also in step SA27, the evaluating apparatus 100 may send the evaluation results to the user client apparatus 200 by using, for example, an existing file transfer technology such as FTP.

The control device 402 in the database apparatus 400 receives the evaluation results or the Web data transmitted from the evaluating apparatus 100 and stores (accumulates) the received evaluation results or the received Web data in a predetermined memory region of the memory device 406 (step SA28).

The receiving part 213 of the client apparatus 200 receives the Web data transmitted from the evaluating apparatus 100, and the received Web data is interpreted with the Web browser 211, to display on the monitor 261 the Web page screen displaying the evaluation results of the individual (step SA29). When the evaluation results are sent from the evaluating apparatus 100 by electronic mail, the electronic mail transmitted from the evaluating apparatus 100 is received at any timing, and the received electronic mail is displayed on the monitor 261 with the known function of the electronic mailer 212 in the client apparatus 200.

In this way, the user can confirm the evaluation results by browsing the Web page displayed on the monitor 261. The user may print out the content of the Web page displayed on the monitor 261 by the printer 262.

When the evaluation results are transmitted by electronic mail from the evaluating apparatus 100, the user reads the electronic mail displayed on the monitor 261, whereby the user can confirm the evaluation results. The user may print out the content of the electronic mail displayed on the monitor 261 by the printer 262.

As described in details above, the client apparatus 200 transmits individual amino acid concentration data to the evaluating apparatus 100. The database apparatus 400 transmits the evaluation formula to the evaluating apparatus 100 in response to a request from the evaluating apparatus 100. The evaluating apparatus 100 then (i) receives the amino acid concentration data from the client apparatus 200 and receives the evaluation formula from the database apparatus 400, (ii) calculates the evaluation value using the received amino acid concentration data and evaluation formula, (iii) estimates the future risk of lifestyle-related disease for the indivisual using the calculated evaluation value and classifies the individual into any one of the plurality of the categories for the future risk of lifestyle-related disease, using the calculated evaluation value and the threshold, and (iv) transmits the obtained evaluation results to the client apparatus 200 and the database apparatus 400. The client apparatus 200 then receives and displays the evaluation results transmitted from the evaluating apparatus 100, and the database apparatus 400 receives and stores the evaluation results transmitted from the evaluating apparatus 100.

In the present description, the evaluating apparatus 100 executes the reception of the amino acid concentration data, the calculation of the value of the evaluation formula, the estimation of the future risk of lifestyle-related disease, the classification of the individual into the category, and the transmission of the evaluation results, while the client apparatus 200 executes the reception of the evaluation results, described as an example. However, when the client apparatus 200 includes the evaluating unit 210a, the evaluating apparatus 100 only has to execute the calculation of the value of the evaluation formula. For example, the conversion of the value of the evaluation formula, the generation of the positional information, the estimation of the future risk of lifestyle-related disease, and the classification of the individual into the category may be appropriately shared between the evaluating apparatus 100 and the client apparatus 200.

For example, when the client apparatus 200 receives the value of the formula from the evaluating apparatus 100, the evaluating unit 210a may convert the value of the formula in the converting unit 210a2, estimate the future risk of lifestyle-related disease using the value of the formula or the converted value, generate the positional information corresponding to the value of the formula or the converted value in the generating unit 210a3, and classify the individual into any one of the plurality of categories for the future risk of lifestyle-related disease using the value of the formula or the converted value in the classifying unit 210a4.

When the client apparatus 200 receives the converted value from the evaluating apparatus 100, the evaluating unit 210a may estimate the future risk of lifestyle-related disease using the converted value, generate the positional information corresponding to the converted value in the generating unit 210a3, and classify the individual into any one of the plurality of categories for the future risk of lifestyle-related disease using the converted value in the classifying unit 210a4.

When the client apparatus 200 receives the value of the formula or the converted value and the positional information from the evaluating apparatus 100, the evaluating unit 210a may estimate the future risk of lifestyle-related disease using the value of the formula or the converted value and classify the individual into any one of categories for the future risk of lifestyle-related disease using the value of the formula or the converted value in the classifying unit 210a4.

### 2-4. Other Embodiments

In addition to the second embodiment described above, the evaluating apparatus, the evaluating method, the evaluating program product, the evaluating system, and the terminal apparatus according to the present invention can be practiced in various different embodiments within the technological scope of the claims.

Of the processings described in the second embodiment, all or a part of the processings described as automatically performed ones may be manually performed, or all or a part of the processings described as manually performed ones may be also automatically performed by known methods.

In addition, the processing procedures, the control procedures, the specific names, the information including parameters such as registered data of various processings and retrieval conditions, the screen examples, and the database configuration shown in the description and the drawings may be arbitrarily modified unless otherwise specified.

The components of the evaluating apparatus 100 shown in the figures are functionally conceptual and therefore not be physically configured as shown in the figures.

For example, for the operational functions provided in the evaluating apparatus 100, in particular, for the operational functions performed in the control device 102, all or part thereof may be implemented by a CPU (Central Processing Unit) and programs interpreted and executed in the CPU, or may be implemented by wired-logic hardware. The program is recorded in a non-transitory computer-readable recording medium including programmed instructions for making an information processing apparatus execute the evaluating method according to the present invention, and is mechanically read as needed by the evaluating apparatus 100. More specifically, computer programs to give instructions to the CPU in cooperation with an OS (operating system) to perform various processes are recorded in the memory device 106 such as ROM or a HDD (hard disk drive). The computer programs are executed by being loaded to RAM, and form the control unit in cooperation with the CPU.

The computer programs may be stored in an application program server connected to the evaluating apparatus 100 via an arbitrary network, and all or part thereof can be downloaded as necessary.

The evaluating program according to the present invention may be stored in the non-transitory computer-readable recording medium, or can be configured as a program product. The “recording medium” mentioned here includes any “portable physical medium” such as a memory card, a USB (universal serial bus) memory, an SD (secure digital) card, a flexible disk, a magneto-optical disc, ROM, EPROM (erasable programmable read only memory), EEPROM (registered trademark) (electronically erasable and programmable read only memory), CD-ROM (compact disk read only memory), MO (magneto-optical disk), DVD (digital versatile disk), and a Blu-ray (registered trademark) Disc.

The “program” mentioned here is a data processing method described in an arbitrary language or description method, and therefore any form such as a source code and a binary code is acceptable. The “program” is not necessarily limited to a program configured as a single unit, and, therefore, includes those dispersively configured as a plurality of modules and libraries and those in which the function of the program is achieved in cooperation with separate programs represented as OS (operating system). Any known configuration and procedures can be used as a specific configuration and reading procedure to read a recording medium by each apparatus shown in the embodiments or as an installation procedure after the reading, or the like.

The various databases and the like stored in the memory device 106 is a storage unit which is a memory device such as RAM and ROM, a fixed disk drive such as a hard disk, a flexible disk, and an optical disc, or the like. The memory device 106 stores therein various programs, tables, databases, files for Web (World Wide Web) pages, and the like used to perform various processes and to provide Web sites.

The evaluating apparatus 100 may be configured as an information processing apparatus such as known personal computer and work station, or may be configured as the information processing apparatus connected to an arbitrary peripheral device. The evaluating apparatus 100 may be provided by installing software (including the programs and the data, etc.) to cause the information processing apparatus to implement the evaluating method according to the present invention.

Furthermore, a specific configuration of dispersion or integration of the apparatuses is not limited to the shown one. The apparatuses can be configured by functionally or physically dispersing or integrating all or part of the apparatuses in arbitrary units according to various types of additions or the like or according to functional loads. In other words, the embodiments may be implemented in arbitrary combinations thereof or an embodiment may be selectively implemented.

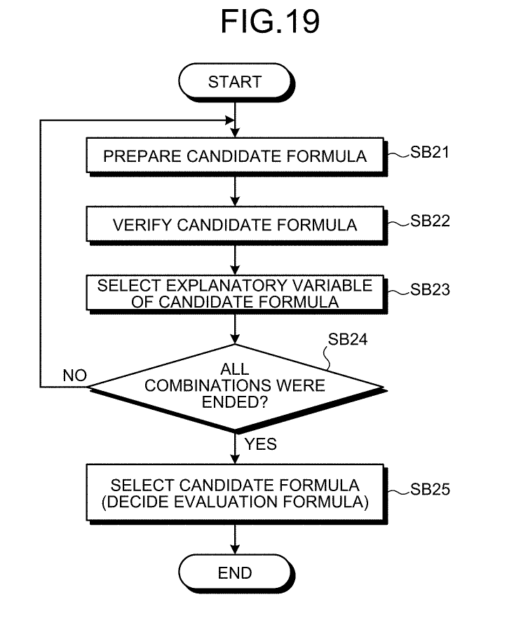

Finally, an example of the evaluation formula-preparing processing performed in the evaluating apparatus 100 is described in detail with reference to FIG. 19. The processing described below is merely one example, and the method of preparing evaluation formula is not limited thereto. FIG. 19 is a flowchart showing an example of the evaluation formula-preparing processing. The evaluation formula-preparing processing may be performed in the database apparatus 400 handling the index state information.

In the present description, the evaluating apparatus 100 stores the index state information previously obtained from the database apparatus 400 in a predetermined memory region of the index state information file 106c. The evaluating apparatus 100 shall store, in a predetermined memory region of the designated index state information file 106d, the index state information including the lifestyle-related disease index data and the amino acid concentration data (the one including the concentration values of the 21 kinds of amino acids) designated previously in the index state information-designating part 102g.

The candidate formula-preparing part 102h1 in the evaluation formula-preparing part 102h first prepares the candidate formulae based on a predetermined formula-preparing method from the index state information stored in a predetermine memory region of the designated index state information file 106d, and stores the prepared candidate formulae in a predetermined memory region of the candidate formula file 106e1 (step SB21). Specifically, the candidate formula-preparing part 102h1 in the evaluation formula-preparing part 102h first selects a desired method out of a plurality of different formula-preparing methods (including those for multivariate analysis such as principal component analysis, discriminant analysis, support vector machine, multiple regression analysis, logistic regression analysis, k-means method, cluster analysis, and decision tree) and determines the form of the candidate formula to be prepared (the form of formula) based on the selected formula-preparing method. The candidate formula-preparing part 102h1 in the evaluation formula-preparing part 102h then performs various calculation corresponding to the selected formula-selecting method (e.g., average or variance), based on the index state information. The candidate formula-preparing part 102h1 in the evaluation formula-preparing part 102h then determines the parameters for the calculation result and the determined candidate formula. In this way, the candidate formula is generated based on the selected formula-preparing method. When the candidate formulae are generated simultaneously and concurrently (in parallel) by using a plurality of different formula-preparing methods in combination, the processings described above may be executed concurrently for each selected formula-preparing method. Alternatively when the candidate formulae are generated in series by using a plurality of different formula-preparing methods in combination, for example, the candidate formulae may be generated by converting the index state information with the candidate formulae prepared by performing principal component analysis and performing discriminant analysis of the converted index state information.

The candidate formula-verifying part 102h2 in the evaluation formula-preparing part 102h verifies (mutually verifies) the candidate formula prepared in step SB21 according to a particular verifying method and stores the verification result in a predetermined memory region of the verification result file 106e2 (step SB22). Specifically, the candidate formula-verifying part 102h2 in the evaluation formula-preparing part 102h first generates the verification data to be used in verification of the candidate formula, based on the index state information stored in a predetermined memory region of the designated index state information file 106d, and verifies the candidate formula according to the generated verification data. If a plurality of the candidate formulae is generated by using a plurality of different formula-preparing methods in step SB21, the candidate formula-verifying part 102h2 in the evaluation formula-preparing part 102h verifies each candidate formula corresponding to each formula-preparing method according to a particular verifying method. Here in step SB22, at least one of the discrimination rate, sensitivity, specificity, information criterion, ROC_AUC (area under the curve in a receiver operating characteristic curve), and the like of the candidate formula may be verified based on at least one method of the bootstrap method, holdout method, N-fold method, leave-one-out method, and the like. Thus, it is possible to select the candidate formula higher in predictability or reliability, by taking the index state information and evaluation condition into consideration.

Then, the explanatory variable-selecting part 102h3 in the evaluation formula-preparing part 102h selects a combination of the amino acid concentration data contained in the index state information used in preparing the candidate formula by selecting an explanatory variable of the candidate formula according to a predetermined explanatory variable-selecting method, and stores the index state information including the selected combination of the amino acid concentration data in a predetermined memory region of the selected index state information file 106e3 (step SB23). When a plurality of the candidate formulae is generated by using a plurality of different formula-preparing methods in step SB21 and each candidate formula corresponding to each formula-preparing method is verified according to a predetermined verifying method in step SB22, the explanatory variable-selecting part 102h3 in the evaluation formula-preparing part 102h may select the explanatory variable of the candidate formula for each candidate formula according to a predetermined explanatory variable-selecting method in step SB23. Here in step SB23, the explanatory variable of the candidate formula may be selected from the verification results according to at least one of the stepwise method, best path method, local search method, and genetic algorithm. The best path method is a method of selecting an explanatory variable by optimizing an evaluation index of the candidate formula while eliminating the explanatory variables contained in the candidate formula one by one. In step SB23, the explanatory variable-selecting part 102h3 in the evaluation formula-preparing part 102h may select a combination of the amino acid concentration data based on the index state information stored in a predetermined memory region of the designated index state information file 106d.

The evaluation formula-preparing part 102h then judges whether all combinations of the amino acid concentration data contained in the index state information stored in a predetermined memory region of the designated index state information file 106d are processed, and if the judgment result is “End” (Yes in step SB24), the processing advances to the next step (step SB25), and if the judgment result is not “End” (No in step SB24), it returns to step SB21. The evaluation formula-preparing part 102h may judge whether the processing is performed a predetermined number of times, and if the judgment result is “End” (Yes in step SB24), the processing may advance to the next step (step SB25), and if the judgment result is not “End” (No in step SB24), it may return to step SB21. The evaluation formula-preparing part 102h may judge whether the combination of the amino acid concentration data selected in step SB23 is the same as the combination of the amino acid concentration data contained in the index state information stored in a predetermined memory region of the designated index state information file 106d or the combination of the amino acid concentration data selected in the previous step SB23, and if the judgment result is “the same” (Yes in step SB24), the processing may advance to the next step (step SB25) and if the judgment result is not “the same” (No in step SB24), it may return to step SB21. If the verification result is specifically the evaluation value for each candidate formula, the evaluation formula-preparing part 102h may advance to step SB25 or return to step SB21, based on the comparison of the evaluation value with a particular threshold corresponding to each formula-preparing method.

Then, the evaluation formula-preparing part 102h determines the evaluation formula by selecting the candidate formula used as the evaluation formula based on the verification results from a plurality of the candidate formulae, and stores the determined formula (the selected candidate formula) in particular memory region of the evaluation formula file 106e4 (step SB25). Here, in step SB25, for example, there are cases where the optimal evaluation formula is selected from the candidate formulae prepared in the same formula-preparing method or the optimal evaluation formula is selected from all candidate formulae.

Given the foregoing description, the explanation of the evaluation formula-preparing processing is finished.

### Example 1

Background data of examinees measured in health screening and data of amino acid concentrations in blood samples taken in the health screening are obtained (in total, 7685 people). To normally distribute and standardize the amino acid concentrations in blood, the following method is conducted. To begin with, from 7685 health screening examinees (4694 males, 2991 females), a reference population of 3885 people (1970 males, 1915 females) are selected based on the following exclusion criteria based on academic society guidelines and the like. Specifically, the reference population is determined by excluding (1) persons regularly receiving drug therapy for chronic disease, (2) persons diagnosed as being in an abnormal level, or having anemia or inflammation in medical checkups (specifically, persons matching at least one of the following conditions on test values), (3) persons whose amino acid concentration in plasma record a high value or a low value by four standard deviations (SD) or more. Distributions of amino acid concentration data by gender of these 3885 people are obtained as follows.

The test values of TP are equal to or smaller than 6.3 g/dl, or are equal to or greater than 8.4 g/dl.
The test values of Alb are equal to or smaller than 3.7 g/dl, or are equal to or greater than 5.3 g/dl.
The test values of T-Bil are equal to or greater than 2.0 mg/dl.
The test values of WBC are equal to or smaller than 1.5×10³/mm³.
The test values of RBC are equal to or smaller than 330×10⁴/mm³.
The test values of Hb are equal to or smaller than 10 g/dl.
The test values of MCV are equal to or smaller than 70 fl.
The test values of UA are equal to or smaller than 1.5 mg/dl, or are equal to or greater than 9.0 mg/dl.
The test values of TG are equal to or greater than 300 mg/dl.
The test values of T-cho are equal to or greater than 300 mg/dl.
The test values of Glucose are equal to or greater than 121 mg/dl.
The test values of γGT are equal to or greater than 100 U/L.
The test values of ALT are equal to or greater than 60 U/L.
The test values of CK are equal to or greater than 350 U/L.
The test values of CRP are equal to or greater than 0.8 mg/dl.
The test values of BMI are equal to or smaller than 14, or are equal to or greater than 30.

TABLE 1

| | Male Lower | Male | Male Upper |
|---|---|---|---|
| | Limit | Median | Limit |
| | 2.5 Percentiles | 50 Percentiles | 97.5 Percentiles |
| Glu | 19.1 | 42.9 | 79.2 |
| Ser | 81.7 | 110.1 | 146.9 |
| Asn | 35.4 | 45.9 | 59.0 |
| Gly | 147.6 | 200.0 | 284.4 |
| Gln | 461.2 | 570.8 | 705.1 |
| His | 66.6 | 82.4 | 101.0 |
| Thr | 87.1 | 122.0 | 170.2 |
| Ala | 234.5 | 350.3 | 493.8 |
| Cit | 20.5 | 30.3 | 43.7 |
| Arg | 64.6 | 90.8 | 124.1 |
| Pro | 93.5 | 141.6 | 236.6 |
| a-ABA | 11.7 | 19.6 | 32.9 |
| Tyr | 47.2 | 63.6 | 86.1 |
| Val | 177.8 | 233.6 | 301.8 |
| Met | 20.2 | 26.9 | 34.5 |
| Orn | 36.0 | 52.5 | 77.1 |
| Lys | 144.3 | 193.0 | 249.7 |
| Ile | 47.3 | 65.5 | 91.0 |
| Leu | 97.6 | 129.8 | 169.5 |
| Phe | 46.8 | 59.1 | 74.6 |
| Trp | 46.2 | 60.3 | 77.7 |

(The unit is μM.)

TABLE 2

| | Female Lower | Female | Female Upper |
|---|---|---|---|
| | Limit | Median | Limit |
| | 2.5 Percentiles | 50 Percentiles | 97.5 Percentiles |
| Glu | 12.5 | 28.8 | 58.9 |
| Ser | 79.0 | 113.7 | 157.0 |
| Asn | 32.8 | 42.9 | 58.9 |
| Gly | 145.8 | 213.5 | 382.8 |
| Gln | 415.6 | 550.8 | 672.2 |
| His | 62.6 | 76.9 | 94.7 |
| Thr | 70.0 | 113.0 | 172.6 |
| Ala | 203.0 | 305.1 | 458.3 |
| Cit | 18.0 | 28.9 | 42.5 |
| Arg | 50.9 | 84.8 | 120.3 |
| Pro | 70.0 | 109.5 | 196.3 |
| a-ABA | 10.7 | 18.1 | 30.9 |
| Tyr | 40.1 | 56.5 | 77.0 |
| Val | 137.8 | 186.6 | 252.2 |
| Met | 17.0 | 22.8 | 30.1 |
| Orn | 25.3 | 45.2 | 71.0 |
| Lys | 119.5 | 171.4 | 226.0 |
| Ile | 35.1 | 48.6 | 68.0 |
| Leu | 73.7 | 98.9 | 129.8 |
| Phe | 42.0 | 52.6 | 67.1 |
| Trp | 39.6 | 52.8 | 68.3 |

(The unit is μM.)

Because the amino acid concentrations are not always normally distributed, Box-Cox transformation is performed by gender for each amino acid to transform the corresponding distribution to a normal distribution. The value λ in the Box-Cox transformation formula below is calculated by a maximum likelihood method.

$${f}_{λ} ( x ) = {\int }_{1}^{x} {t}^{λ - 1} dt + 1 = \{ \begin{matrix}log x + 1 & ( λ = 0 ) \\ \frac{{x}^{λ} - 1}{λ} + 1 & ( λ \neq 0 )\end{matrix} \qquad [ FORMULA 1 ]$$

Subsequently, the resultant distribution is converted with a mean of 50 and a standard deviation of 10, and a formula for converting an amino acid concentration to a standard score (amino acid concentration standard score) is obtained by gender for each amino acid concentration.

### Example 2

Blood samples taken from examinees in health screening and the 120-minute OGTT blood glucose level of the examinees measured in health screening are obtained (in total, 650 people). Blood samples taken from examinees in health screening and the visceral fat area values of the examinees measured in abdominal CT image diagnosis conducted in health screening are obtained (in total, 650 people). Blood samples taken from examinees in health screening and the diagnosis results as to fatty liver by ultrasonography conducted in health screening (the diagnosis results of fatty liver patients (465) and non-fatty liver subjects (1,535)) are obtained (in total, 2,000 people).

From a plurality of multiple regression equations that include, as explanatory variables, “four amino acids Gly, Tyr, Asn, and Ala” and “two amino acids” selected from the 15 kinds of amino acids excluding the four amino acids from the 19 kinds of amino acids(Ala, Arg, Asn, Cit, Gln, Gly, His, Ile, Leu, Lys, Met, Orn, Phe, Pro, Ser, Thr, Trp, Tyr, and Val) using the explanatory variable coverage method in light of correlation to the visceral fat area value and in which the p value in the covariant (age) likelihood ratio test is greater than 0.05, a multiple regression equation with the highest adjusted R-squared is selected. As a result, Index Formula 1 below is selected. From a plurality of logistic regression equations that include, as explanatory variables, “four amino acids Gly, Tyr, Asn, and Ala” and “two amino acids” selected from the 15 kinds of amino acids using the explanatory variable coverage method in light of discriminating between fatty liver and non-fatty liver and in which the p value in the covariant (age) likelihood ratio test is greater than 0.05, a logistic regression equation with the lowest Akaike information criterion is selected. As a result, Index Formula 2 below is selected.

“a₁×Asn+b₁×Gly+c₁×Ala+d₁×Val+e₁×Tyr+f₁×Trp+g₁” Index Formula 1:

“a₂×Asn+b₂×Gly+c₂×Ala+d₂×Cit+e₂×Leu+f₂×Tyr+g₂” Index Formula 2:
In Index Formula 1, a₁, b₁, c₁, d₁, e₁, and f₁ each are a real number other than zero, and g₁ is a real number.
In Index Formula 2, a₂, b₂, c₂, d₂, e₂, and f₂ each are a real number other than zero, and g₂ is a real number.

### Example 3

Persons who took health screening consecutive five years are targeted (4297 people). From among the targeted examinees, examinees who did not develop a disease event on the first year are extracted for each disease event shown in 1. to 41. below. The amino acid concentration standard scores and the values of Index Formulae 1 and 2 (function values) are calculated for each disease event using the amino acid concentrations of the extracted examinees.

For each amino acid concentration, when the corresponding amino acid concentration standard score is lower than the mean−2SD (when the amino acid concentration standard score<30), the amino acid concentration is defined as an amino acid low value (e.g., Glu low value). When the corresponding amino acid concentration standard score is higher than mean+2SD (when the amino acid concentration standard score>70), the amino acid concentration is defined as an amino acid high value (e.g., Glu high value). Out of 10 kinds of amino acids including Arg that is a semiessential amino acid in addition to the essential amino acids (Val, Leu, Ile, Phe, His, Thr, Lys, Met, Trp), when at least one amino acid concentration standard score is lower than the mean−2SD (the amino acid concentration standard score<30), the amino acid concentration is defined as an essential amino acid low value. When at least one amino acid concentration standard score is higher than the mean+2SD (the amino acid concentration standard score>70), the amino acid concentration is defined as an essential amino acid high value.

For each of 41 kinds of disease events described below, an odds ratio for event development within four years is calculated by logistic regression. For amino acid concentration standard scores, all of odds ratios the p values of which are smaller than 0.05 when the deviation increases by 1SD are calculated. For amino acid low values, amino acid high values, essential amino acid low values, essential amino acid high values, odds ratios that are equal to or greater than one and the p values of which are smaller than 0.05, which determine whether to fall under each group, are calculated. For Index Formulae 1 and 2, odds ratios that are equal to or greater than one and the p values of which are smaller than 0.05 when the function value increases by one are calculated.

1. Hypertension
If systolic blood pressure is equal to or greater than 140 mmHg or if diastolic blood pressure is equal to or greater than 90 mmHg, hypertension is diagnosed.

2. Fatty Liver
If evidence of fatty liver is observed based on a liver-to-spleen contrast ratio in an abdominal ultrasound test, fatty liver is diagnosed.

3. High Risk Fatty Liver
If fatty liver is diagnosed and if AST (GOT) is higher than 38 U/l, high-risk fatty liver is diagnosed.

4. Diabetes
If any one of Items 1 to 3 below and Item 4 are identified, diabetes is diagnosed.

Item 1: Early morning fasting blood glucose level equal to or greater than 126 mg/dl.

Item 2: 120-minute 75 g-OGTT blood glucose level equal to or greater than 200 mg/dl.

Item 3: Casual blood glucose level equal to or greater than 200 mg/dl.

Item 4: HbA1C (JDS value) equal to or greater than 6.1% [HbA1C (international standard) equal to or greater than 6.5%].

5. Impaired Glucose Tolerance
If the 120-minute 75 g-OGTT blood glucose level is equal to or greater than 140 mg/dl and equal to or smaller than 199 mg/dl, impaired glucose tolerance is diagnosed.

6. Obesity
If “the waist is equal to or greater than 85 cm for males, equal to or greater than 90 cm for females” (guideline of visceral fat area value being equal to or greater than 100 cm²) or if “the BMI is equal to or greater than 25”, obesity is diagnosed.

7. Morbid Obesity
If the BMI is equal to or greater than 30, morbid obesity is diagnosed.

8. Dyslipidemia
If “triglyceride (TG) is equal to or greater than 150 mg/dl, if HDL cholesterol is less than 40 mg/dl, or if LDL cholesterol is equal to or greater than 140 mg/dl”, dyslipidemia is diagnosed.

9. Chronic Nephropathy
If the estimated glomerular filtration rate (eGFR) is less than 60, chronic nephropathy is diagnosed.

10. Arteriosclerosis
If evidence of hardening is observed in arteriosclerosis screening, arteriosclerosis is diagnosed.

11. Cerebral Infarction
If evidence of cerebral infarction is observed in a head MRI or an MRA test, cerebral infarction is diagnosed.

12. Risk of Heart Disease
If Minnesota code falls out of the normal range, the presence of risk of heart disease is diagnosed.

13. Metabolic Syndrome
If Item 1 below is applicable, when at least two of Items 2 to 4 below are applicable, metabolic syndrome is diagnosed.

Item 1: “Waist equal to or greater than 85 cm for males, equal to or greater than 90 cm for females” (guideline for the visceral fat area value equal to or greater than 100 cm²) or “BMI equal to or greater than 25”.

Item 2: “Triglyceride equal to or greater than 150 mg/dl” and/or “HDL cholesterol less than 40 mg/dl”.

Item 3: “Systolic blood pressure equal to or greater than 130 mmHg” and/or “diastolic blood pressure equal to or greater than 85 mmHg”.

Item 4: “Fasting blood glucose equal to or greater than 110 mg/dl”.

14. Risk of Sympathetic Nerve Disorder

If the heart rate is equal to or higher than 90/min, or if the neutrophil ratio is equal to or higher than 79%, the presence of risk of sympathetic nerve disorder is diagnosed.

15. Risk of Inflammatory Disease

If the CRP value is equal to or larger than 0.3 mg/dl, the presence of risk of inflammatory disease is diagnosed.

16. Risk of Anemia

For males, if the hemoglobin content is equal to or lower than 13.5 g/dl, if the hematocrit value is equal to or smaller than 39.8%, or if the red blood cell count is equal to or lower than 427×10⁴/mm³, the presence of risk of anemia is diagnosed. For females, if the hemoglobin content is equal to or lower than 11.3 g/dl, if the hematocrit value is equal to or smaller than 33.4%, if the red blood cell count is equal to or lower than 376×10⁴/mm³, or if serum iron is equal to or less than 48 μg/dl, the presence of risk of anemia is diagnosed.

17. Risk of Protein Malnutrition

If blood albumin is less than 4 mg/dl, or total blood protein is less than 6.7 mg/dl, the presence of risk of protein malnutrition is diagnosed.

18. Risk of Immune Depression

If the lymphocyte ratio is equal to or lower than 25%, the presence of risk of immune depression is diagnosed.

19. Risk of Physique (Obese Physique)

If the diagnostic result of this item in health screening is “careful attention is required in daily life”, “treatment is required”, “thorough examination is required”, or “treatment continues”, the presence of risk is diagnosed.

20. Risk of Respiratory Disease

If the diagnostic result of this item in health screening is “careful attention is required in daily life”, “treatment is required”, “thorough examination is required”, or “treatment continues”, the presence of risk is diagnosed.

21. Risk of Cardiovascular Disease

If the diagnostic result of this item in health screening is “careful attention is required in daily life”, “treatment is required”, “thorough examination is required”, or “treatment continues”, the presence of risk is diagnosed.

22. Risk of High Blood Pressure

If the diagnostic result of this item in health screening is “careful attention is required in daily life”, “treatment is required”, “thorough examination is required”, or “treatment continues”, the presence of risk is diagnosed.

23. Risk of Kidney And Urinary Tract Disease

If the diagnostic result of this item in health screening is “careful attention is required in daily life”, “treatment is required”, “thorough examination is required”, or “treatment continues”, the presence of risk is diagnosed.

24. Risk of Gastrointestinal Disease

If the diagnostic result of this item in health screening is “careful attention is required in daily life”, “treatment is required”, “thorough examination is required”, or “treatment continues”, the presence of risk is diagnosed.

25. Risk of Liver Disease

If the diagnostic result of this item in health screening is “careful attention is required in daily life”, “treatment is required”, “thorough examination is required”, or “treatment continues”, the presence of risk is diagnosed.

26. Risk of Biliopancreatic Disease

If the diagnostic result of this item in health screening is “careful attention is required in daily life”, “treatment is required”, “thorough examination is required”, or “treatment continues”, the presence of risk is diagnosed.

27. Risk of Carbohydrate Metabolism Disorder

If the diagnostic result of this item in health screening is “careful attention is required in daily life”, “treatment is required”, “thorough examination is required”, or “treatment continues”, the presence of risk is diagnosed.

28. Risk of Lipid Metabolism Disorder

If the diagnostic result of this item in health screening is “careful attention is required in daily life”, “treatment is required”, “thorough examination is required”, or “treatment continues”, the presence of risk is diagnosed.

29. Risk of Uric Acid Metabolism Disorder

If the diagnostic result of this item in health screening is “careful attention is required in daily life”, “treatment is required”, “thorough examination is required”, or “treatment continues”, the presence of risk is diagnosed.

30. Risk of Blood Disease

If the diagnostic result of this item in health screening is “careful attention is required in daily life”, “treatment is required”, “thorough examination is required”, or “treatment continues”, the presence of risk is diagnosed.

31. Risk of Serum Disease

If the diagnostic result of this item in health screening is “careful attention is required in daily life”, “treatment is required”, “thorough examination is required”, or “treatment continues”, the presence of risk is diagnosed.

32. Risk of Eye Disease

If the diagnostic result of this item in health screening is “careful attention is required in daily life”, “treatment is required”, “thorough examination is required”, or “treatment continues”, the presence of risk is diagnosed.

33. Hearing Abnormality

If the diagnostic result of this item in health screening is “careful attention is required in daily life”, “treatment is required”, “thorough examination is required”, or “treatment continues”, the presence of risk is diagnosed.

34. Risk of Urinary System Disease

If the diagnostic result of this item in health screening is “careful attention is required in daily life”, “treatment is required”, “thorough examination is required”, or “treatment continues”, the presence of risk is diagnosed.

35. Height Tumor Marker Levels

If the diagnostic result of this item in health screening is “careful attention is required in daily life”, “treatment is required”, “thorough examination is required”, or “treatment continues”, the presence of risk is diagnosed.

36. Risk of Gynecologic Disease

If the diagnostic result of this item in health screening is “careful attention is required in daily life”, “treatment is required”, “thorough examination is required”, or “treatment continues”, the presence of risk is diagnosed.

37. Risk of Breast Disease

If the diagnostic result of this item in health screening is “careful attention is required in daily life”, “treatment is required”, “thorough examination is required”, or “treatment continues”, the presence of risk is diagnosed.

38. Risk of Encephalopathy

If the diagnostic result of this item in health screening is “careful attention is required in daily life”, “treatment is required”, “thorough examination is required”, or “treatment continues”, the presence of risk is diagnosed.

39. Risk of Arteriosclerosis

If the diagnostic result of this item in health screening is “careful attention is required in daily life”, “treatment is required”, “thorough examination is required”, or “treatment continues”, the presence of risk is diagnosed.

40. Risk of Reduction in Bone Mineral Density

If the diagnostic result of this item in health screening is “careful attention is required in daily life”, “treatment is required”, “thorough examination is required”, or “treatment continues”, the presence of risk is diagnosed.

41. Risk of Other Disease

If the diagnostic result of this item in health screening is “careful attention is required in daily life”, “treatment is required”, “thorough examination is required”, or “treatment continues”, the presence of risk is diagnosed.

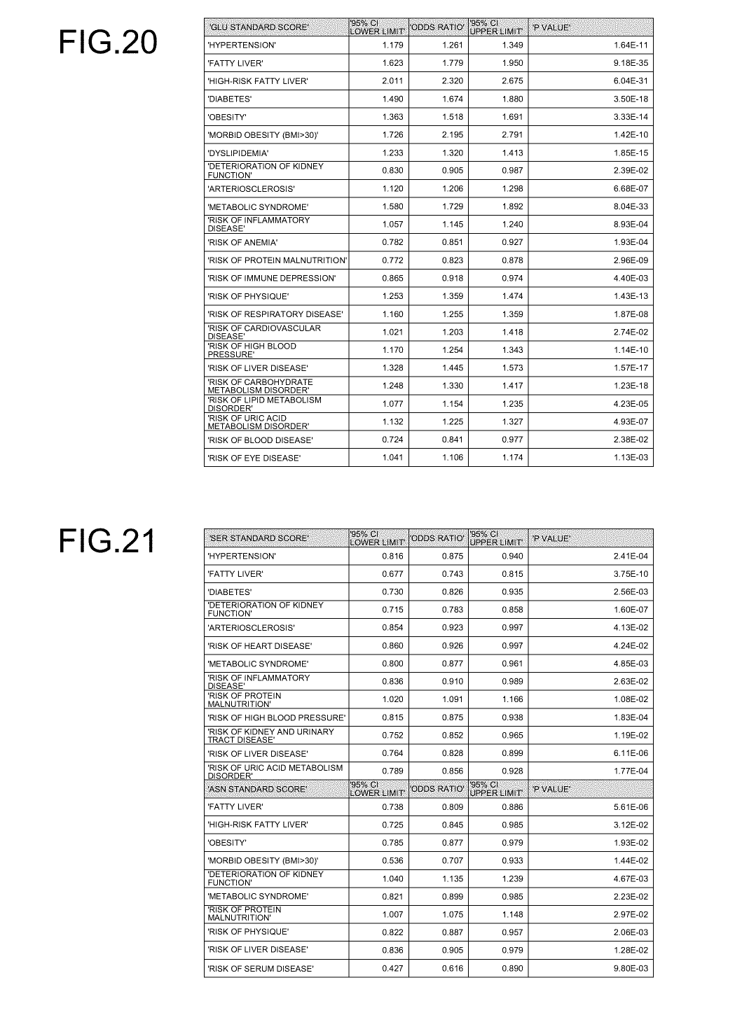

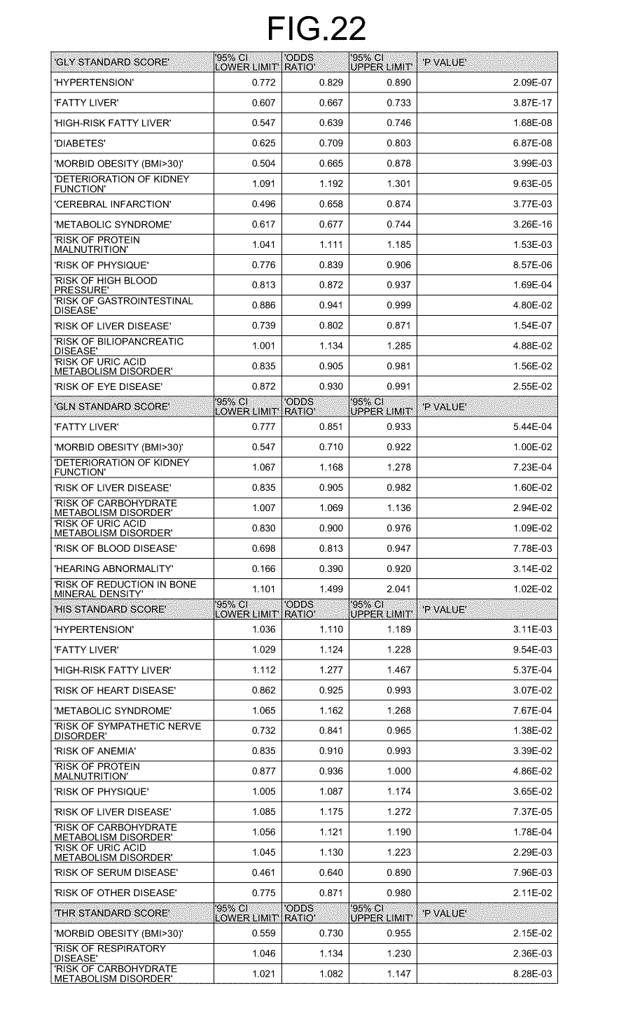

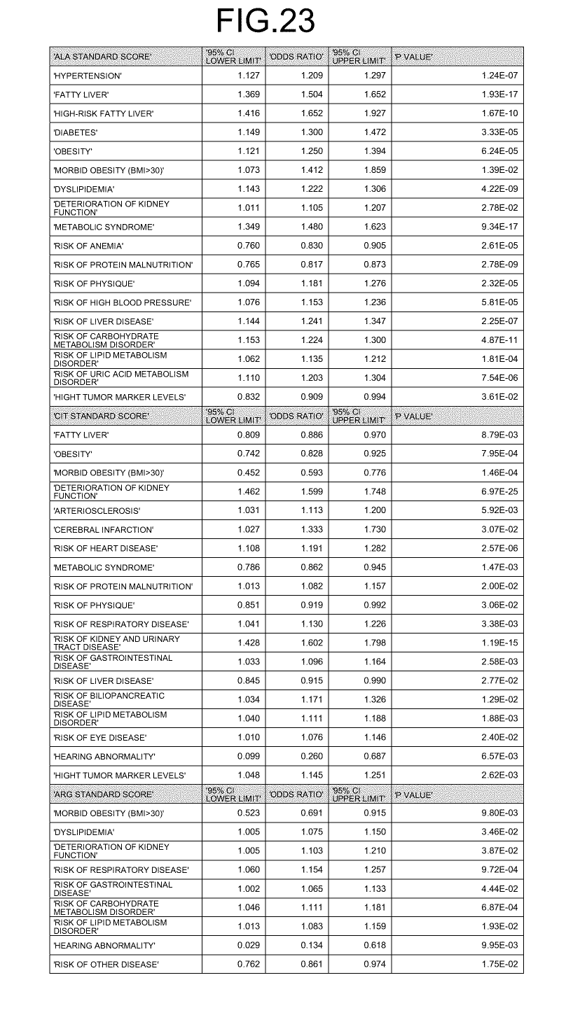

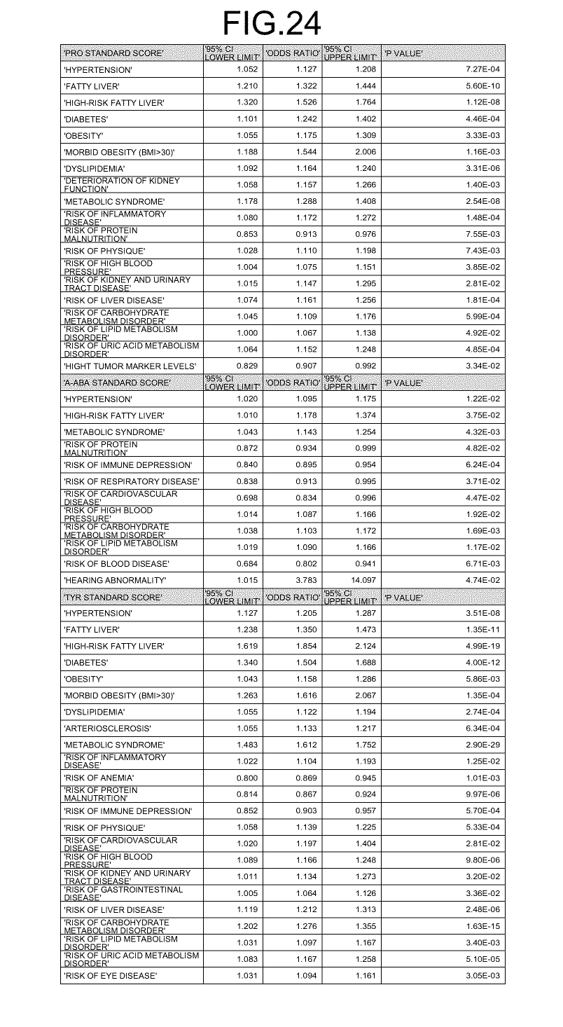

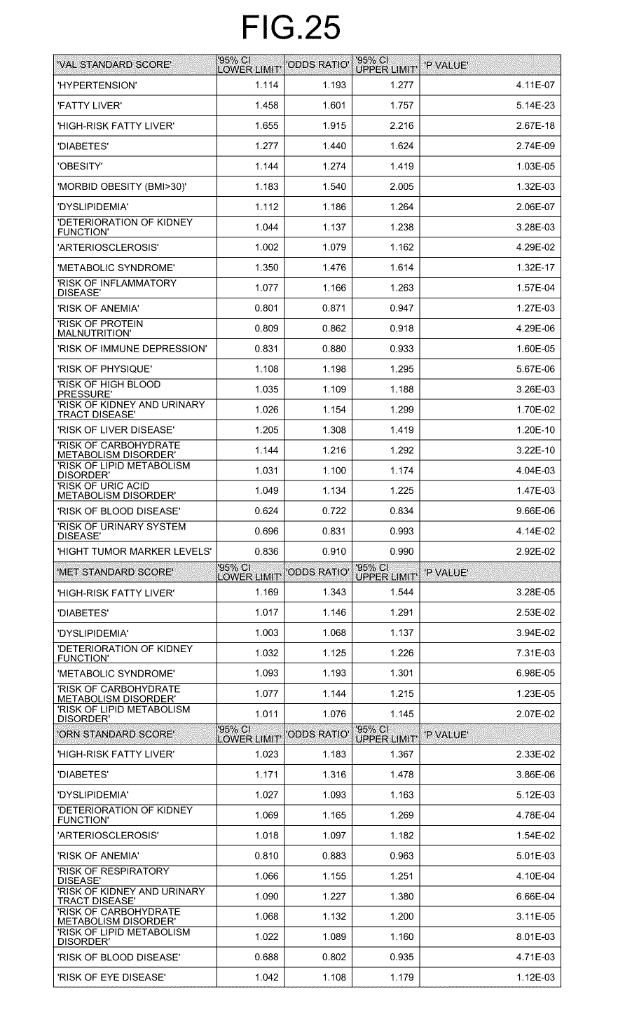

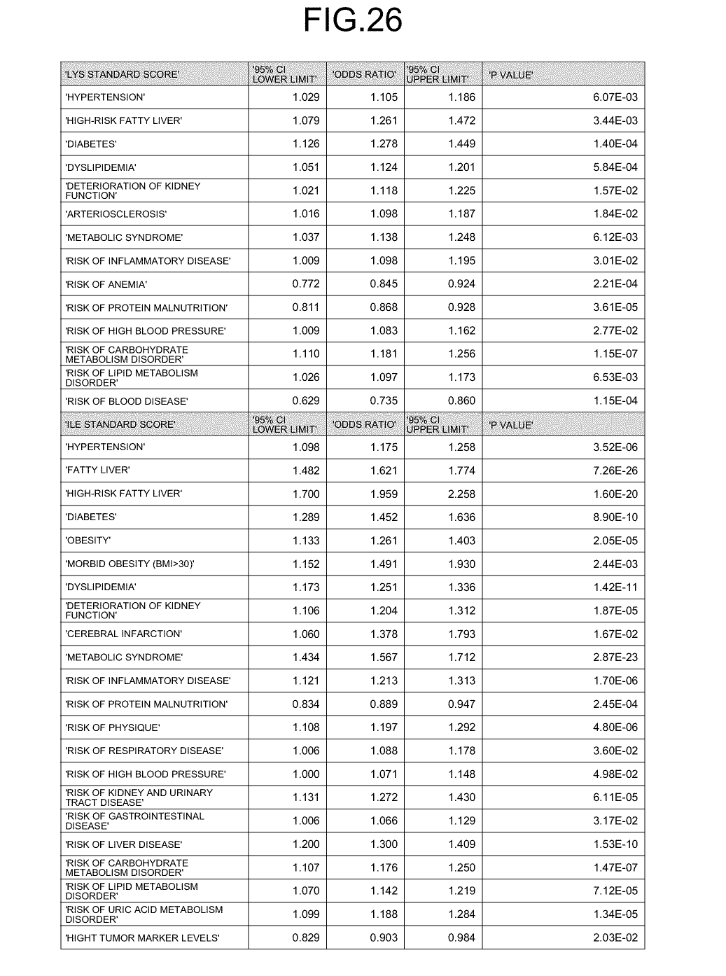

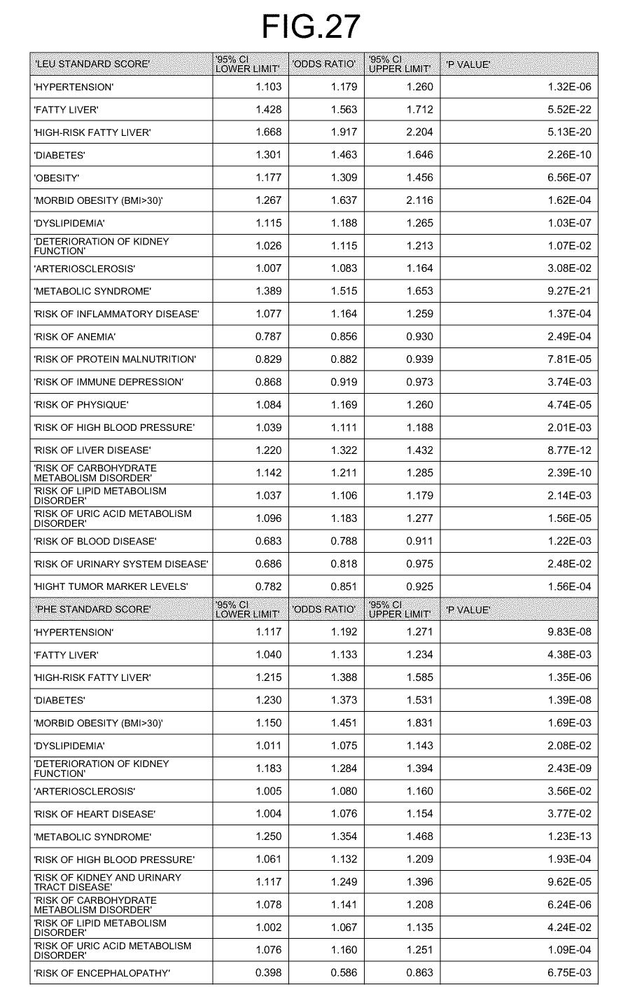

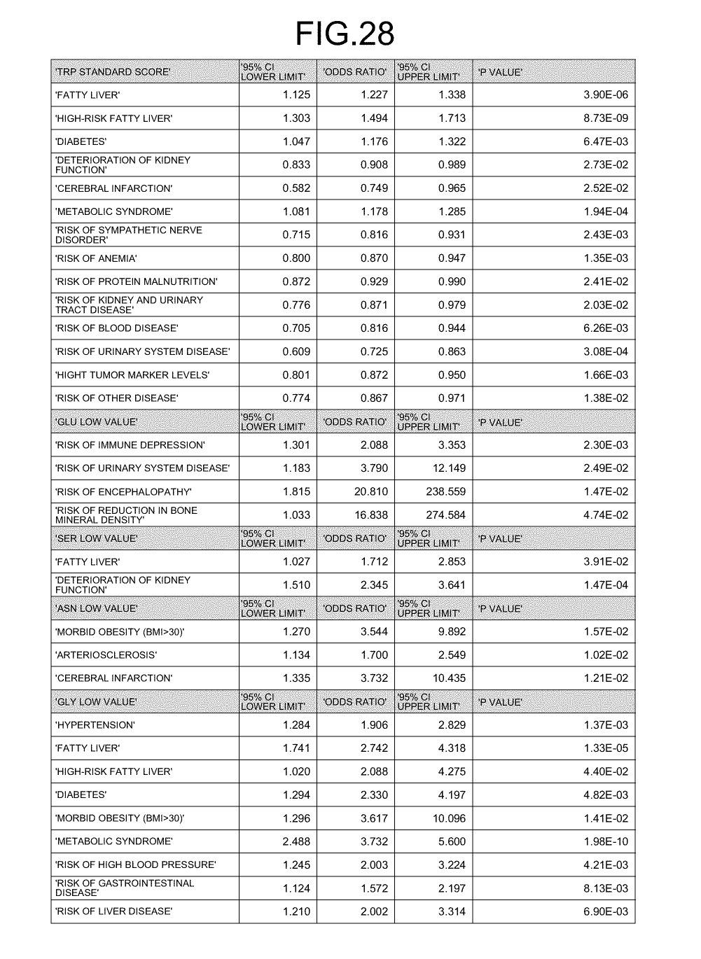

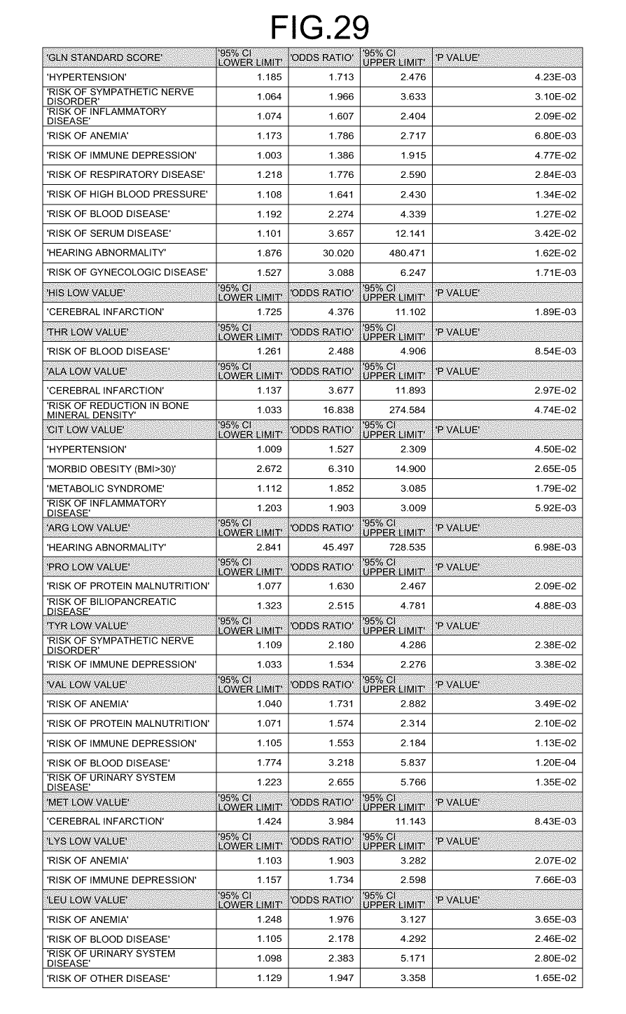

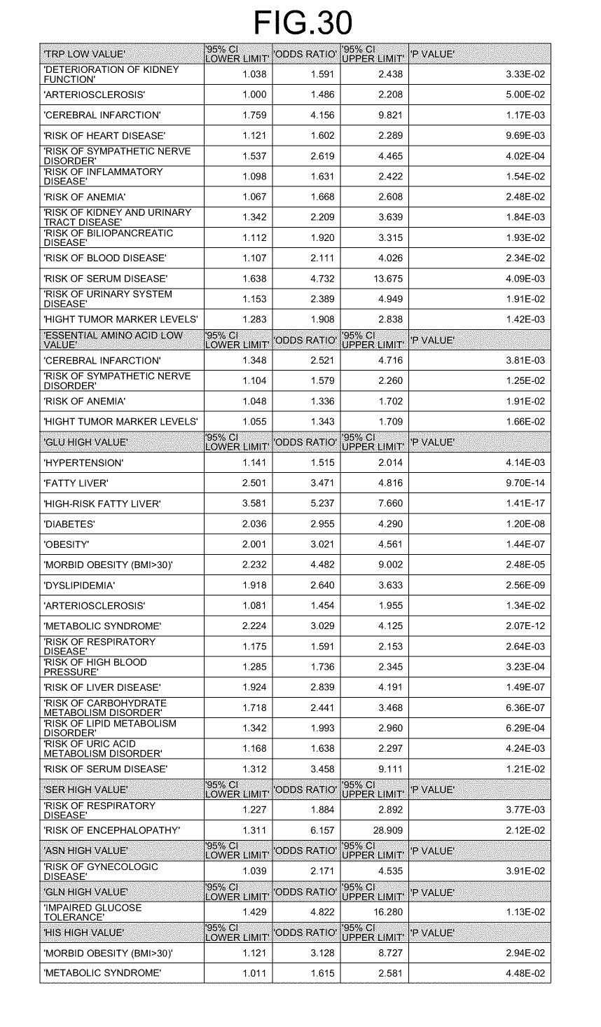

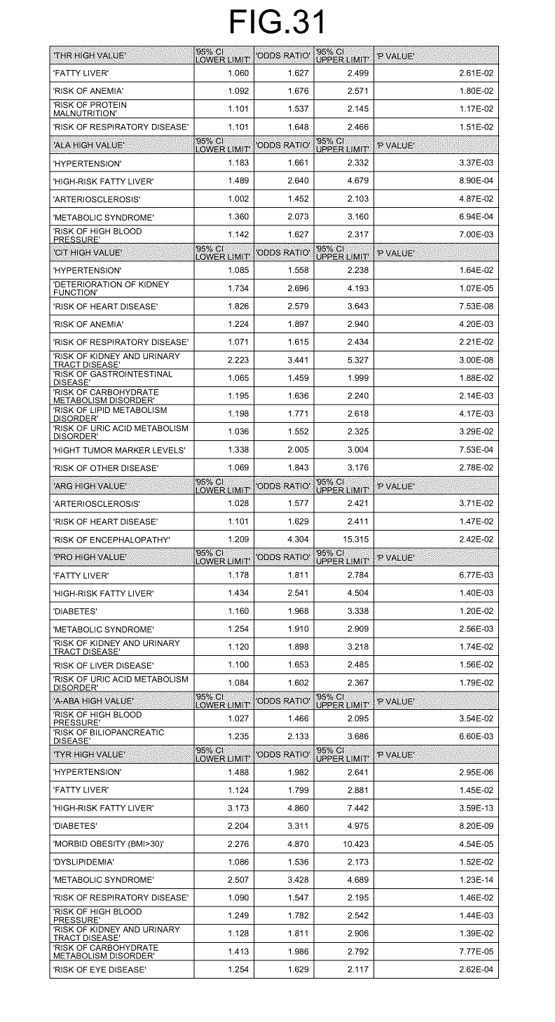

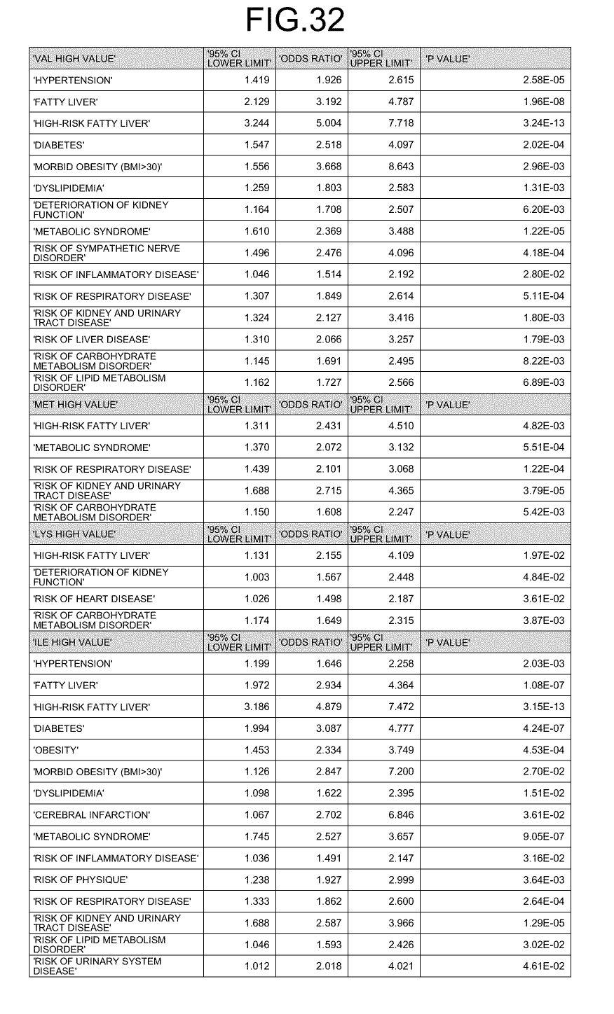

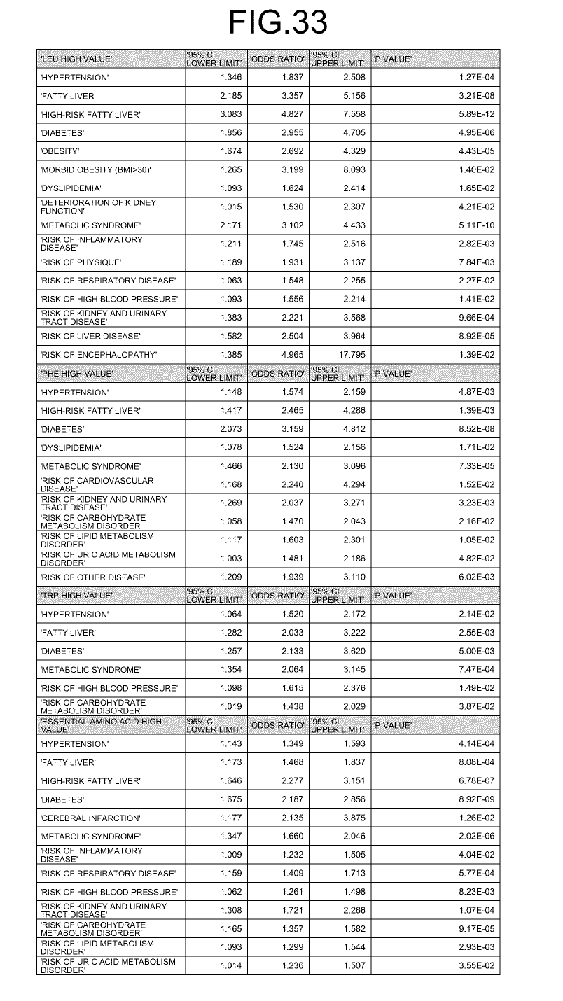

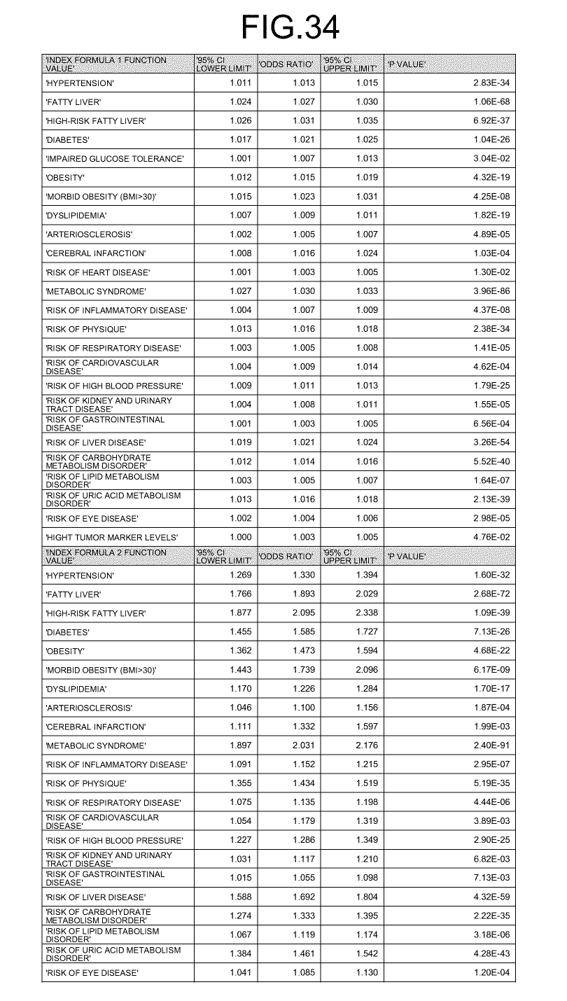

In FIG. 20 to FIG. 34, odds ratios when background factors are not adjusted, 95%-confidence interval lower limits and 95%-confidence interval upper limits thereof, and p values (p<0.05) of the odds ratios are each listed.

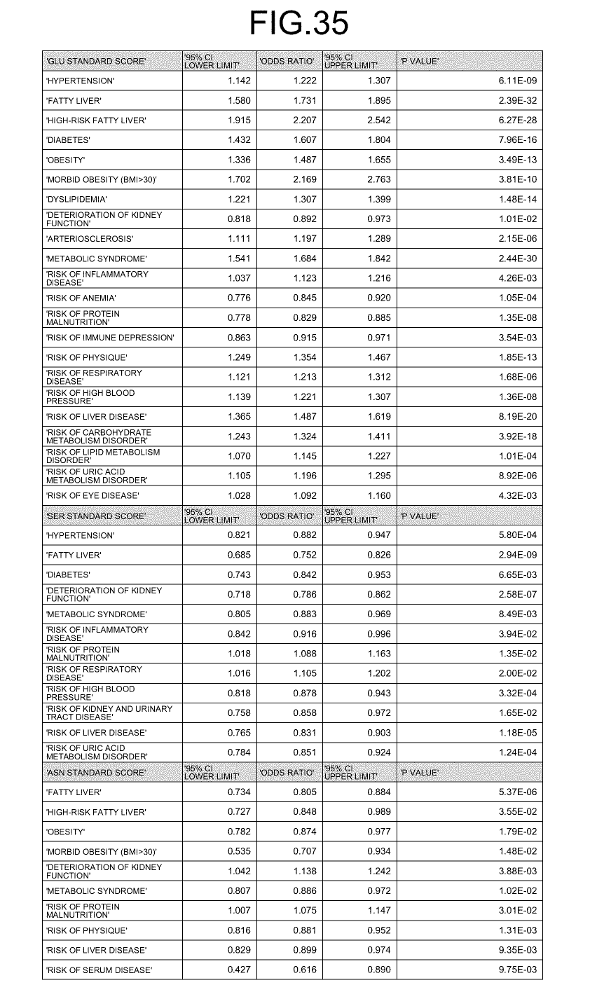

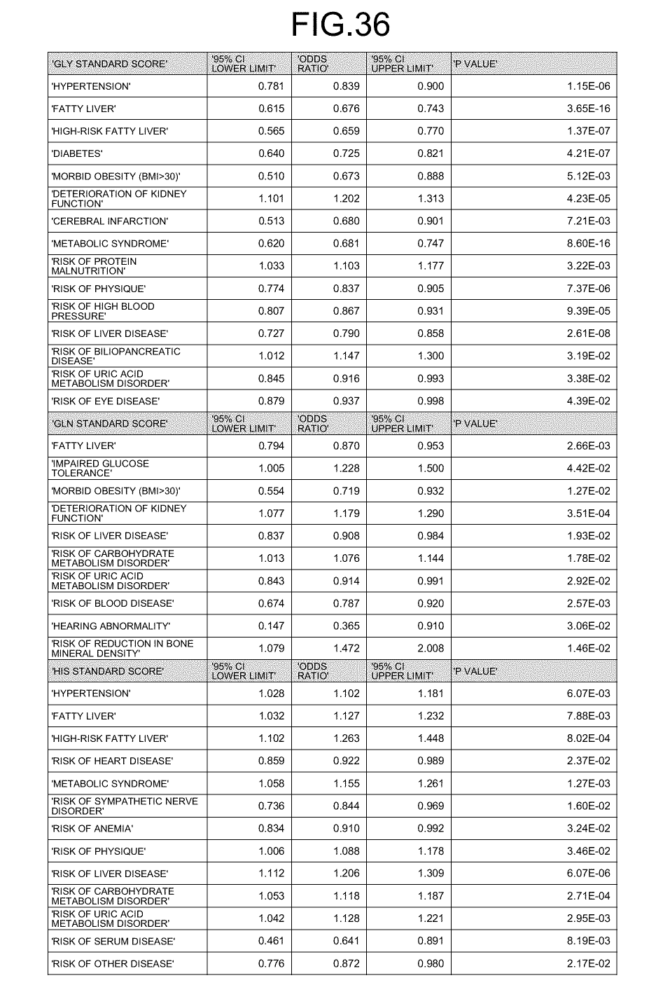

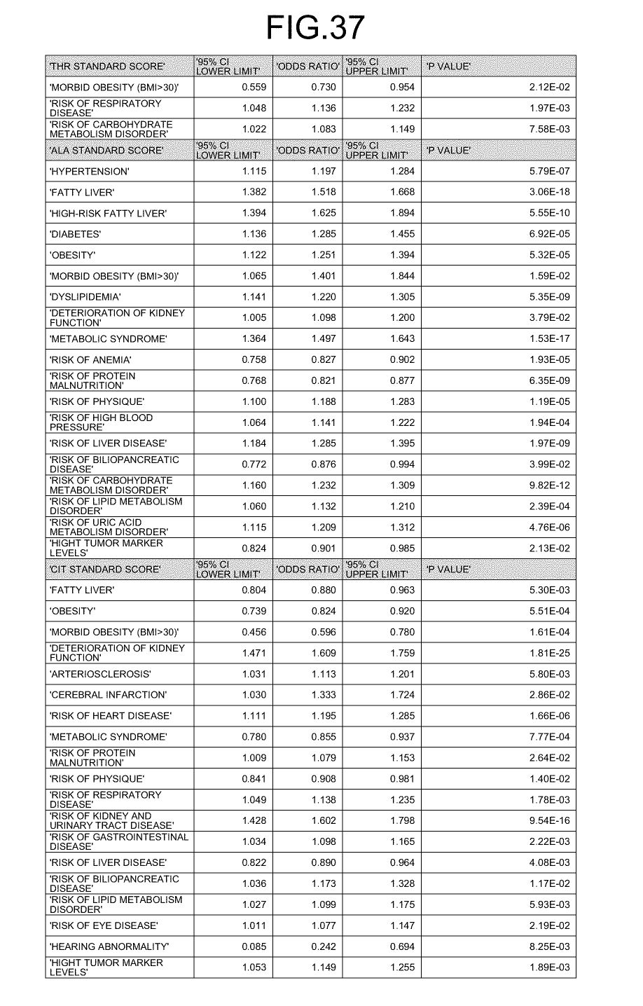

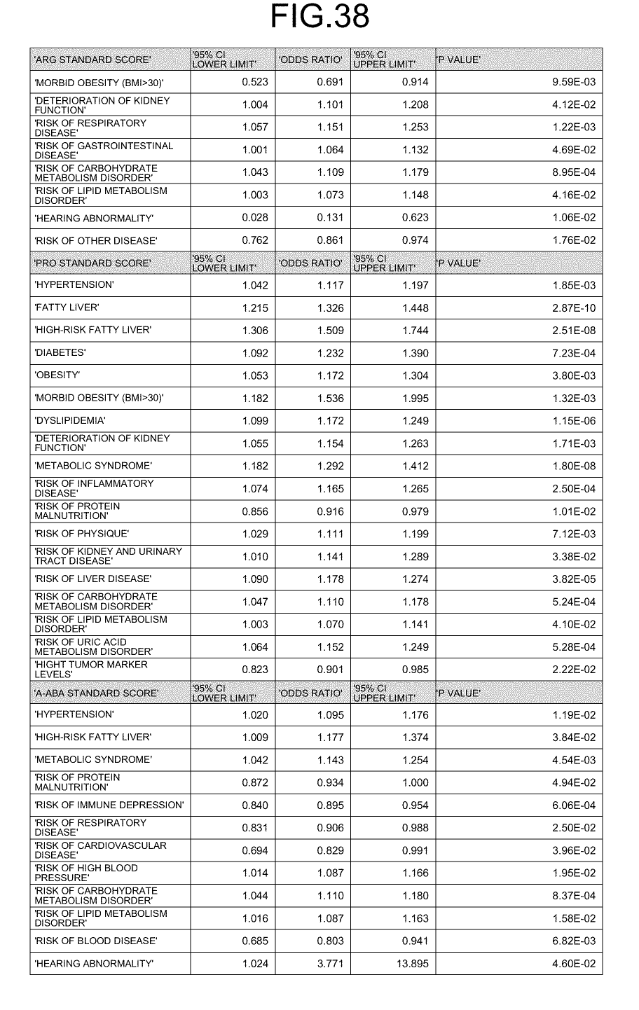

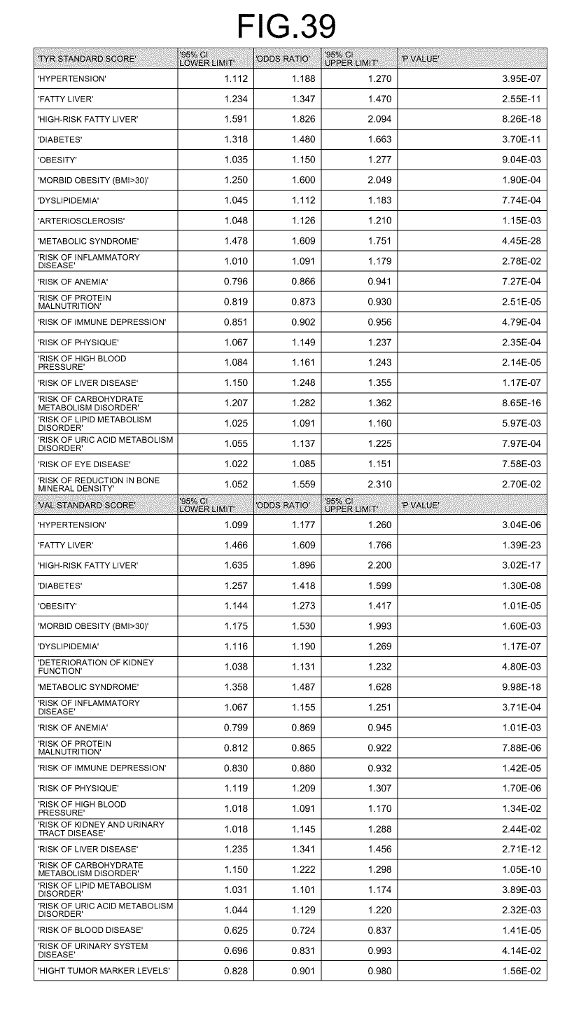

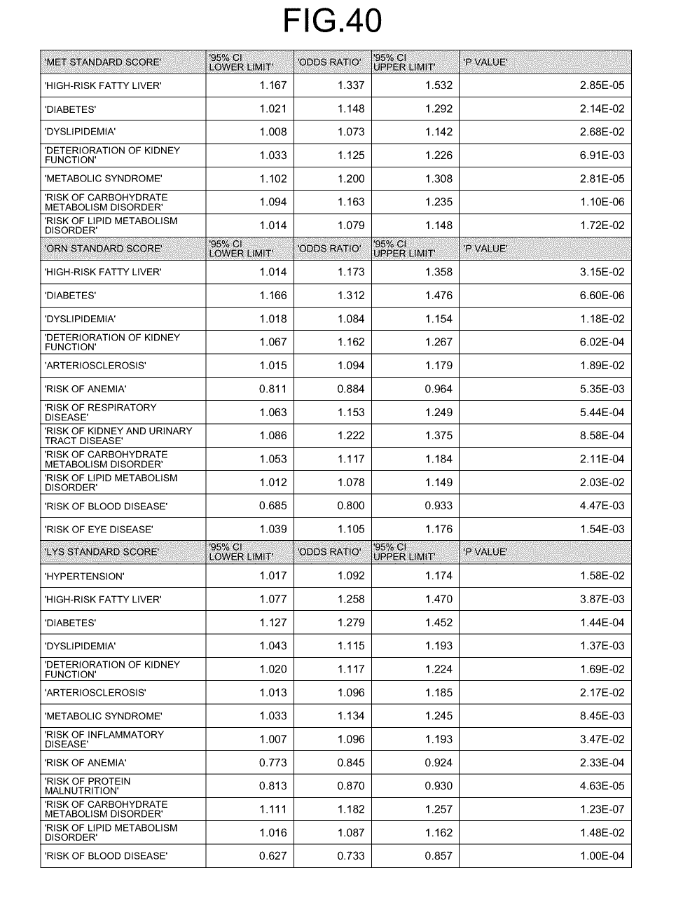

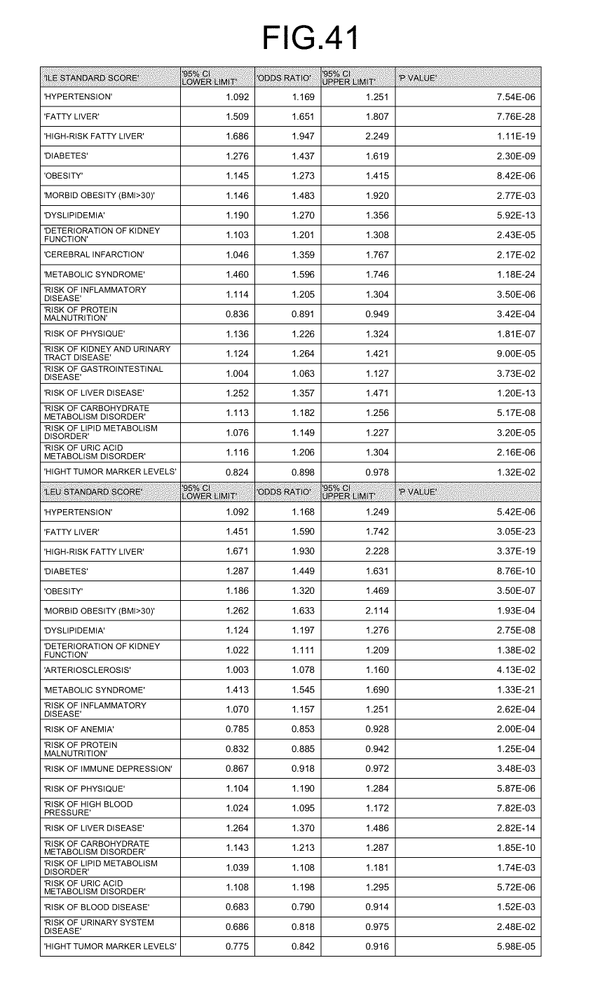

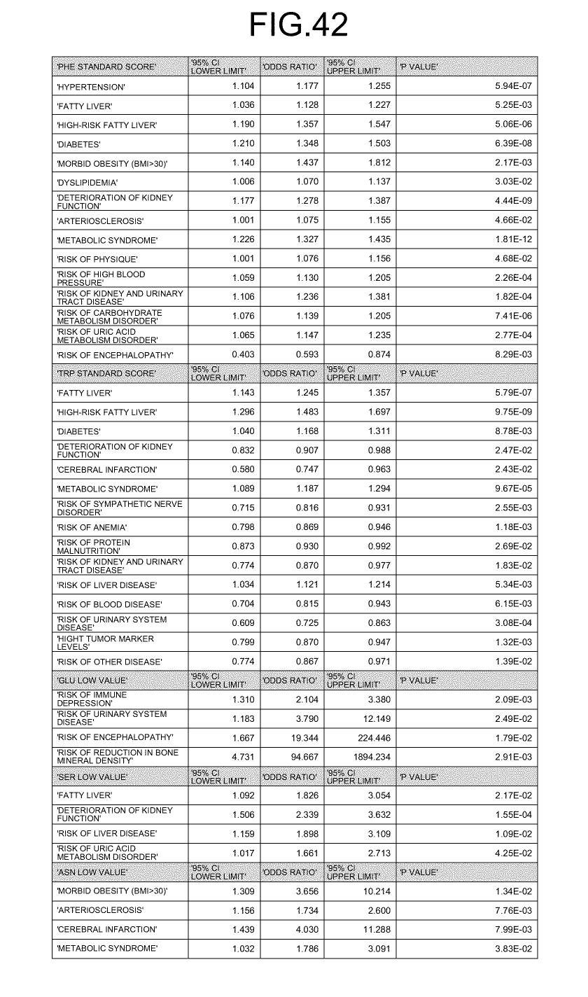

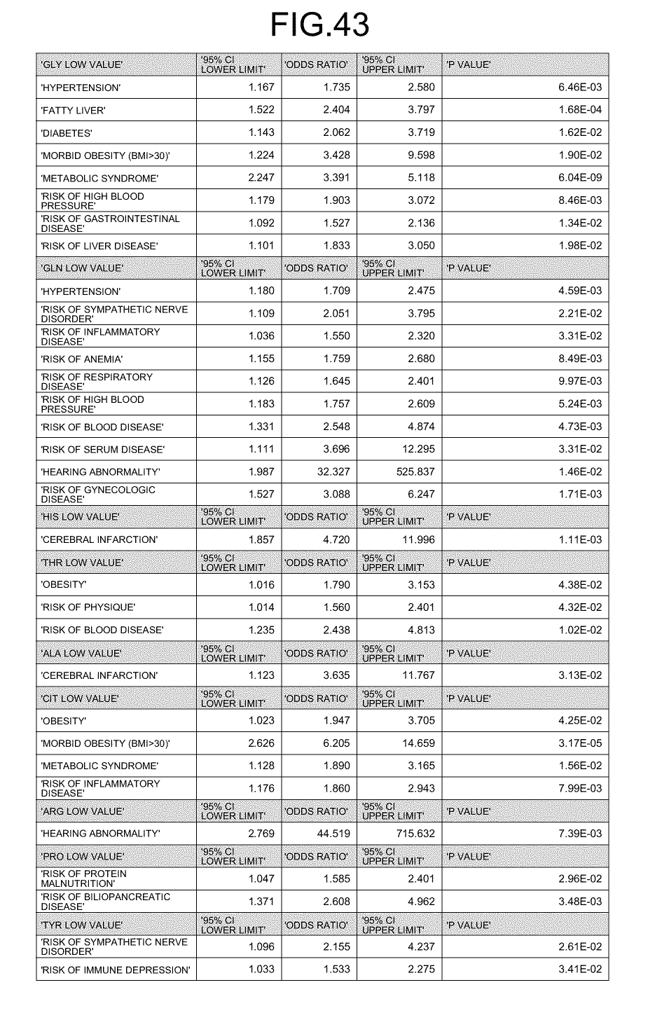

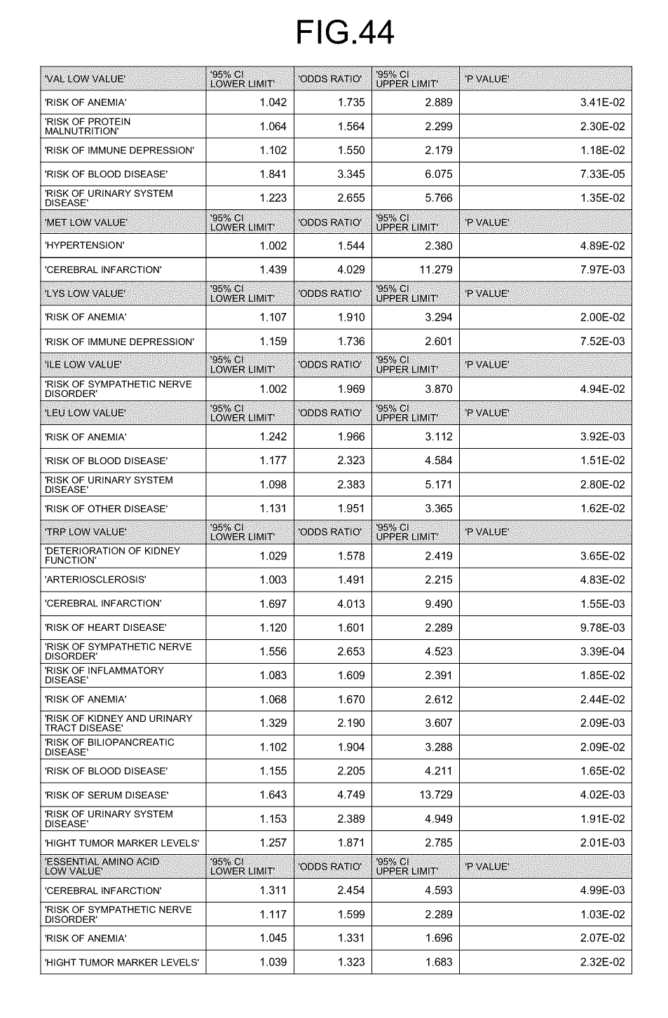

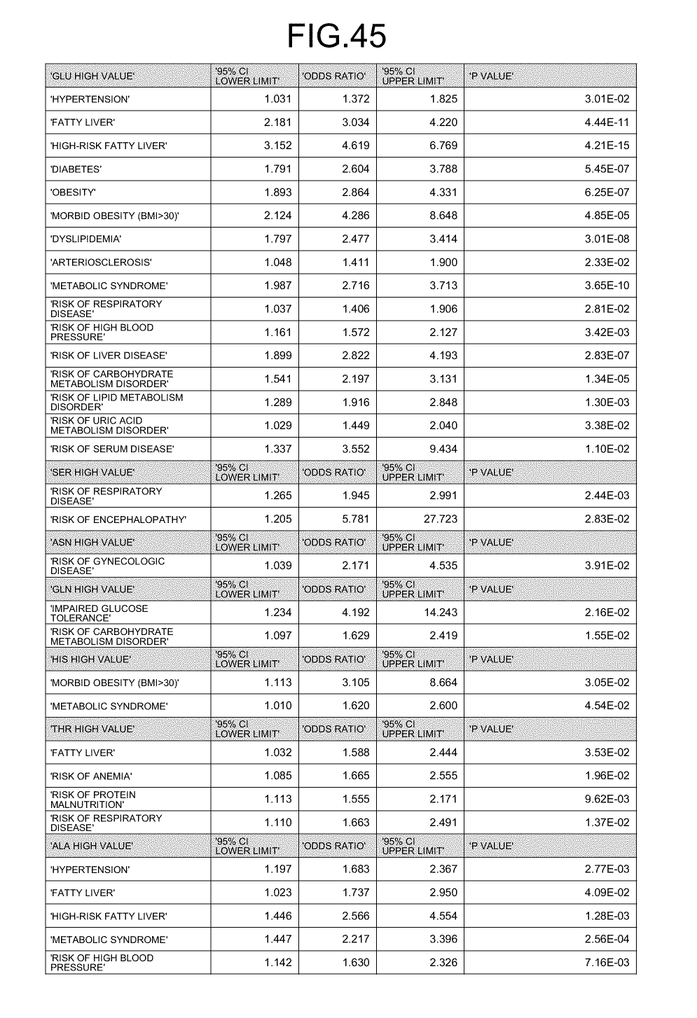

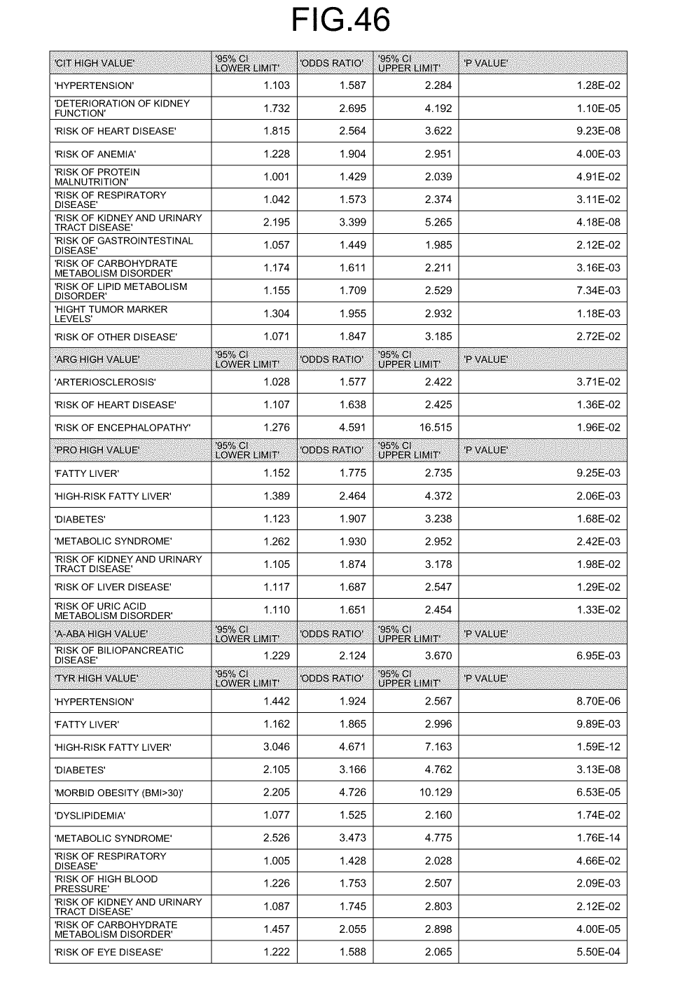

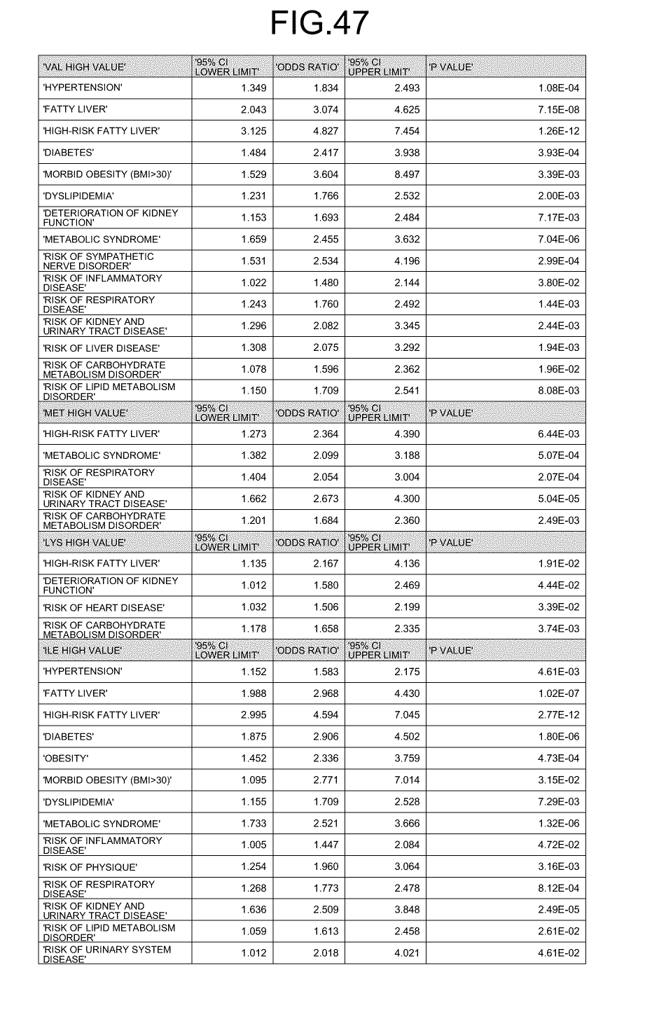

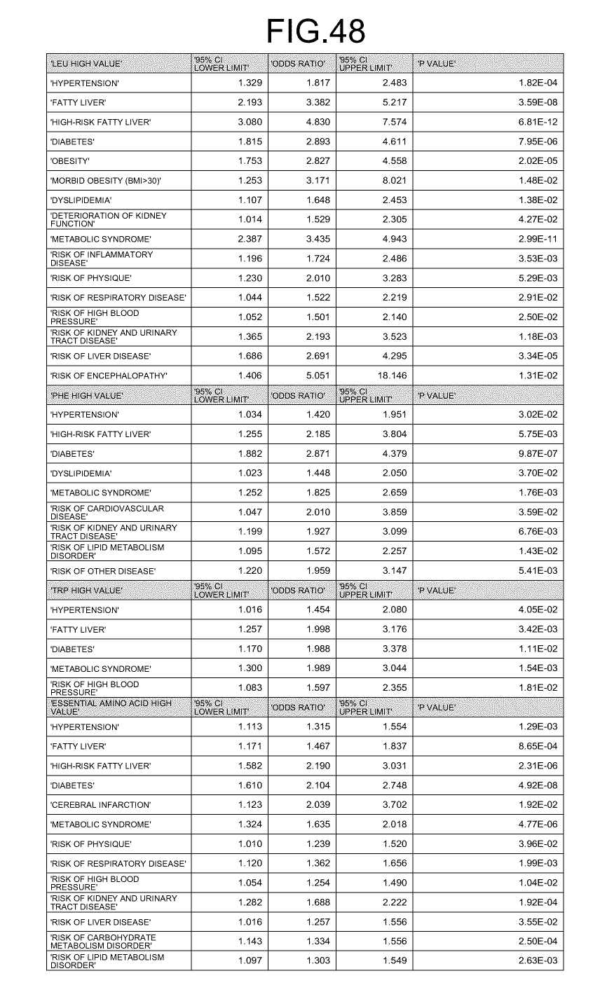

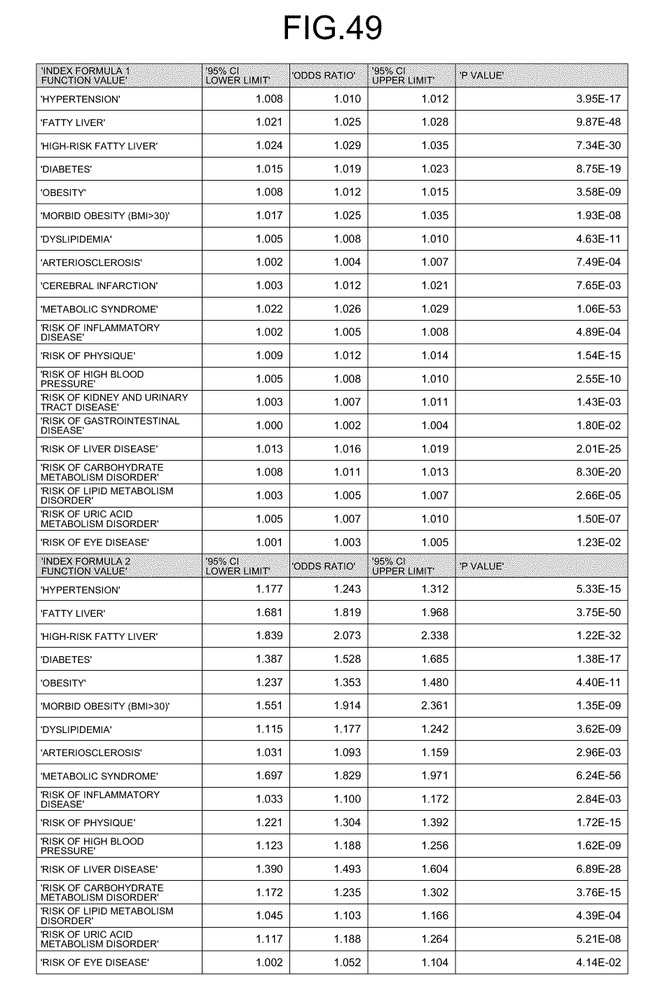

In FIG. 35 to FIG. 49, odds ratios when gender as a background factor is adjusted, 95%-confidence interval lower limits and 95%-confidence interval upper limits thereof, and p values (p<0.05) of the odds ratios are each listed.

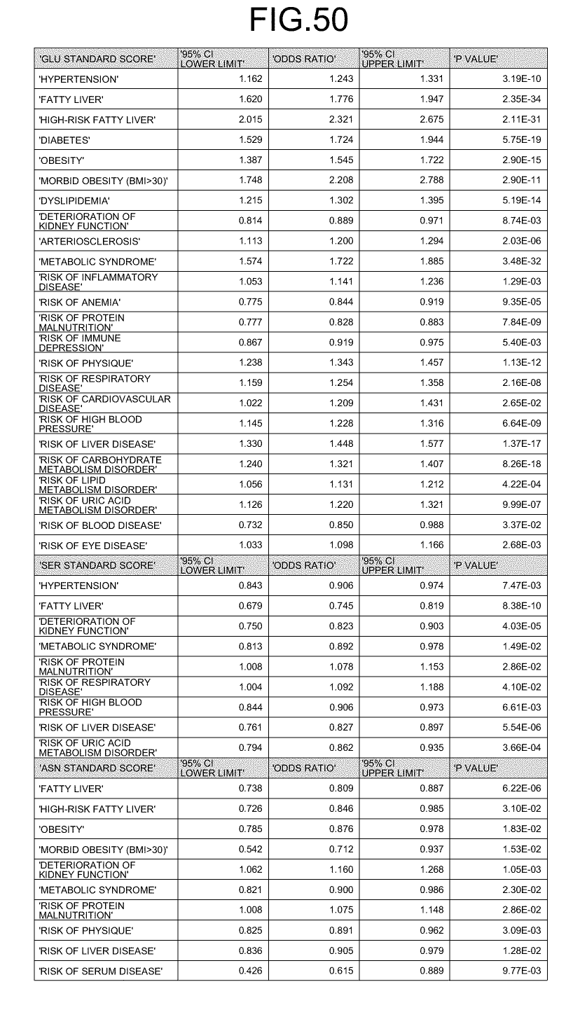

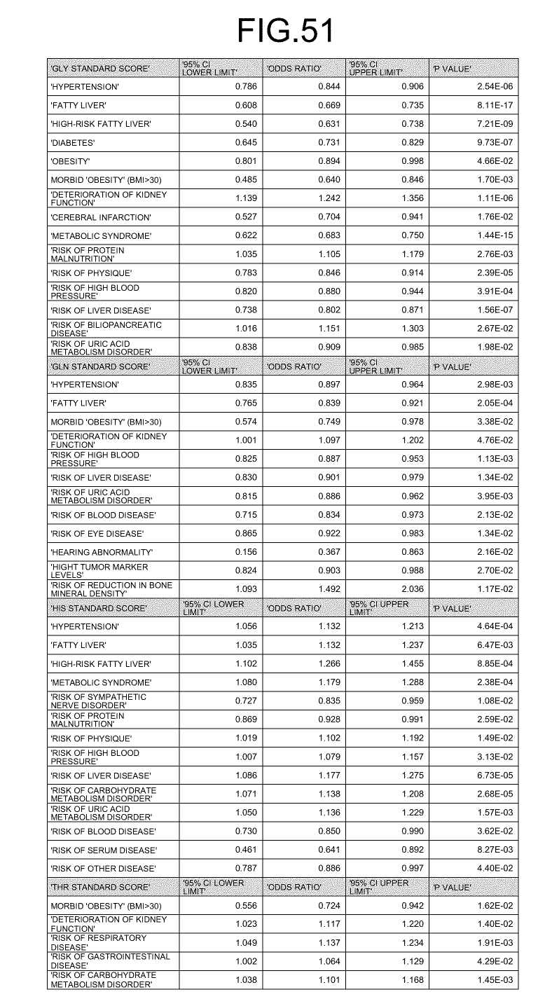

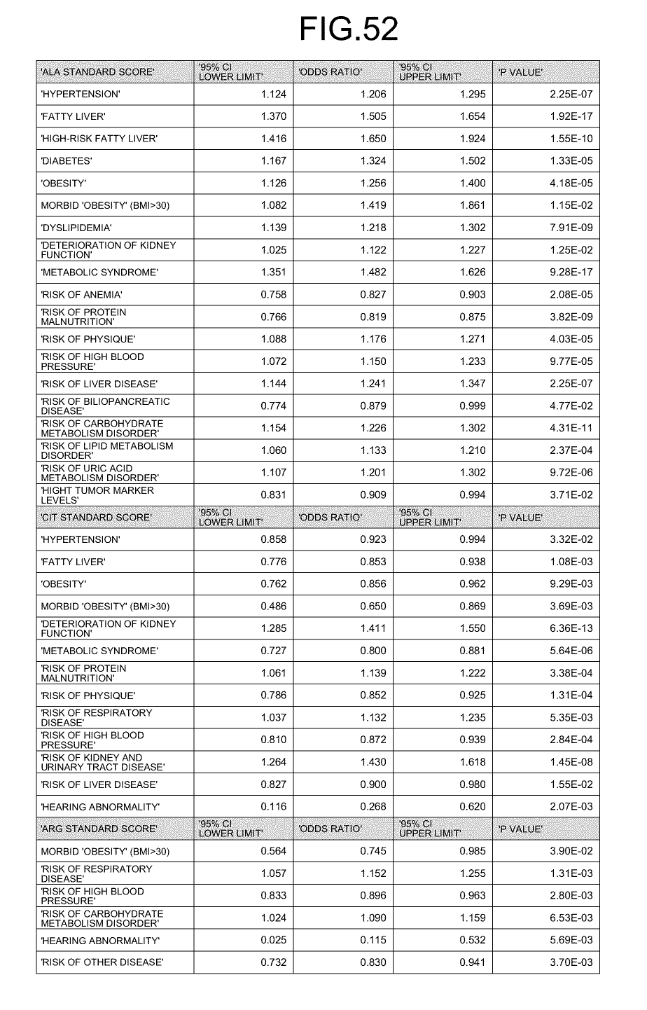

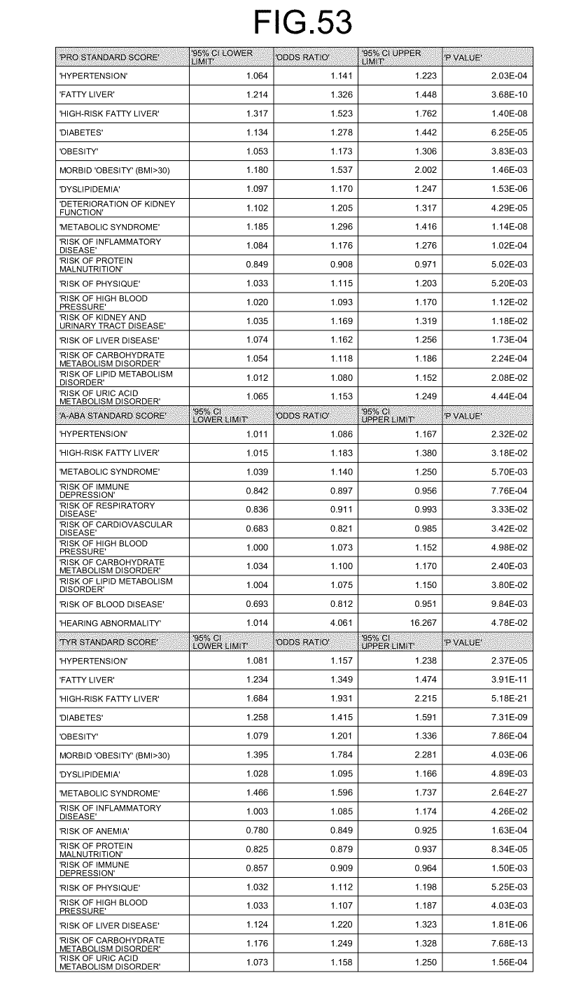

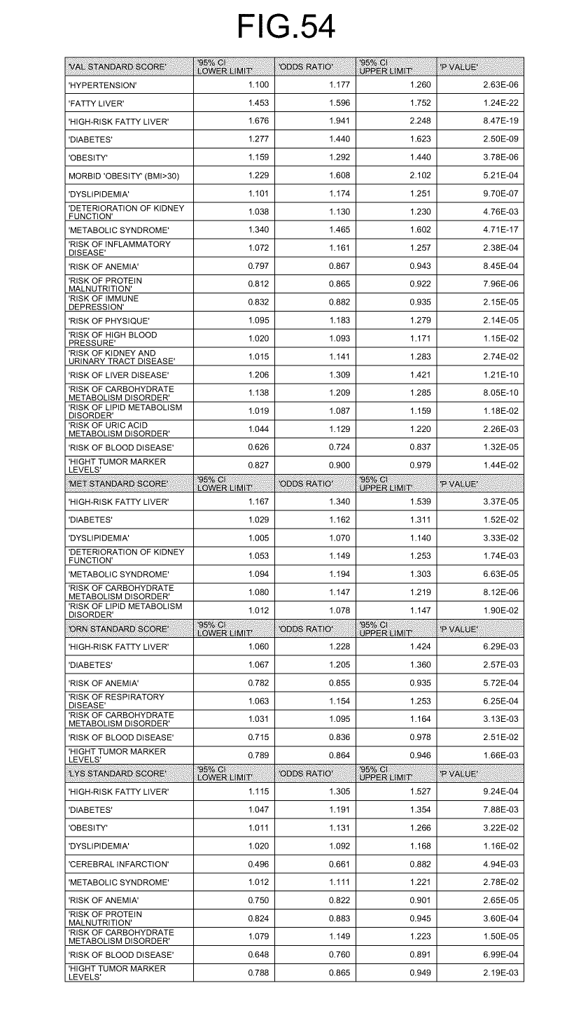

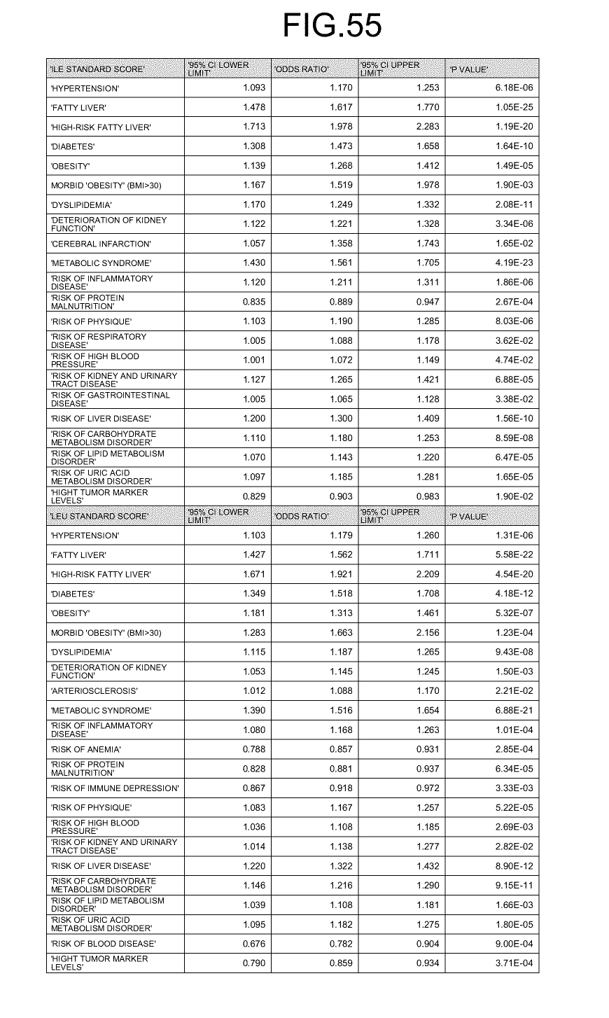

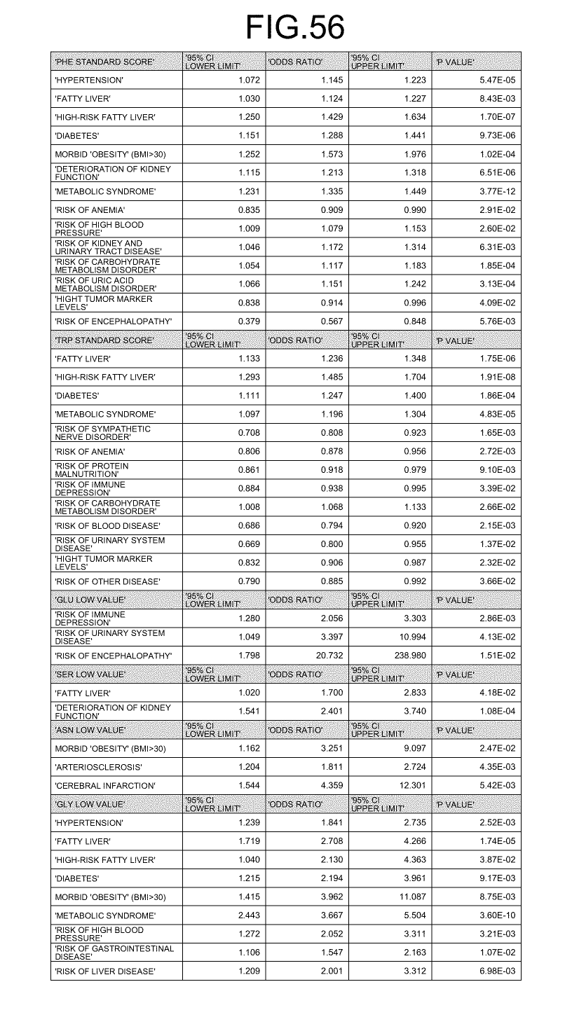

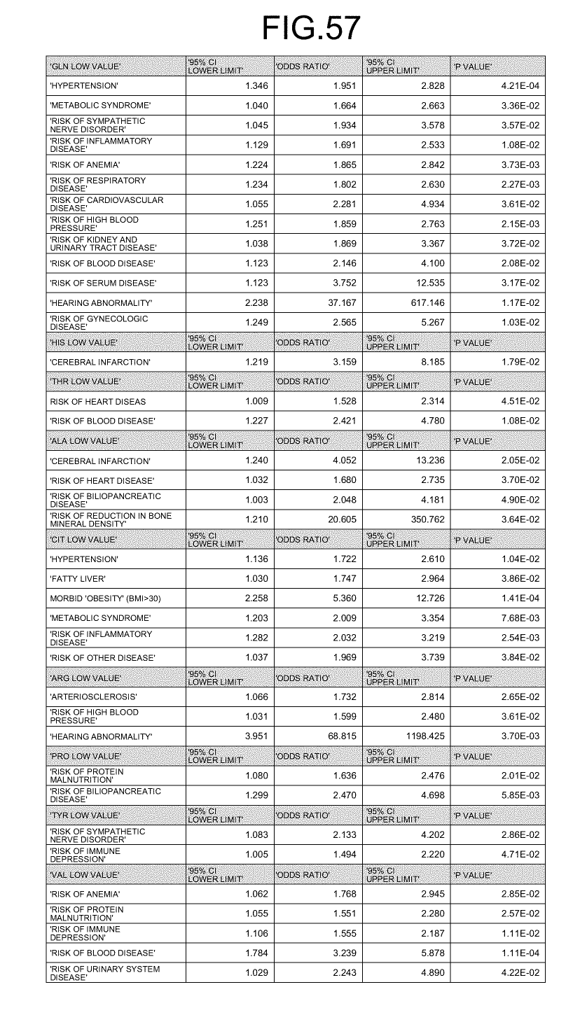

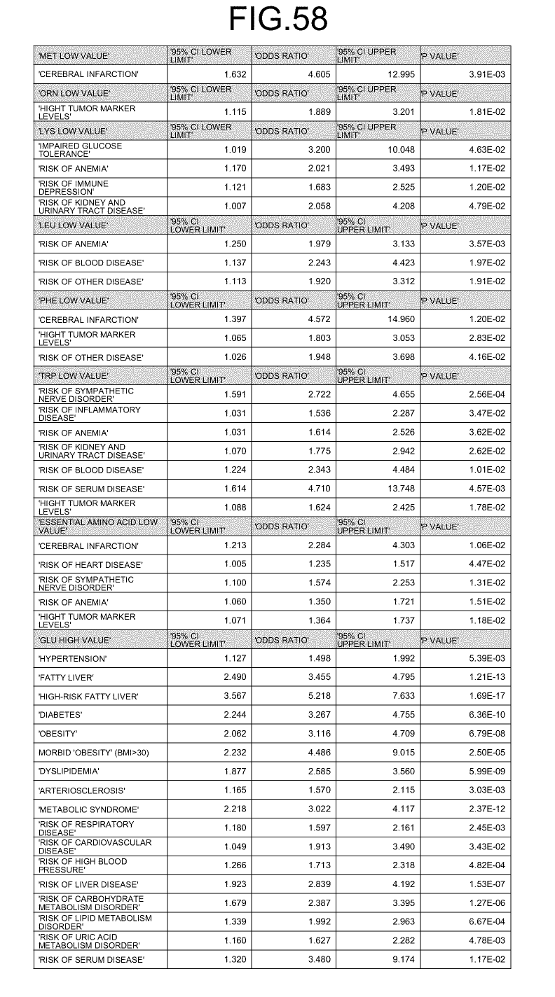

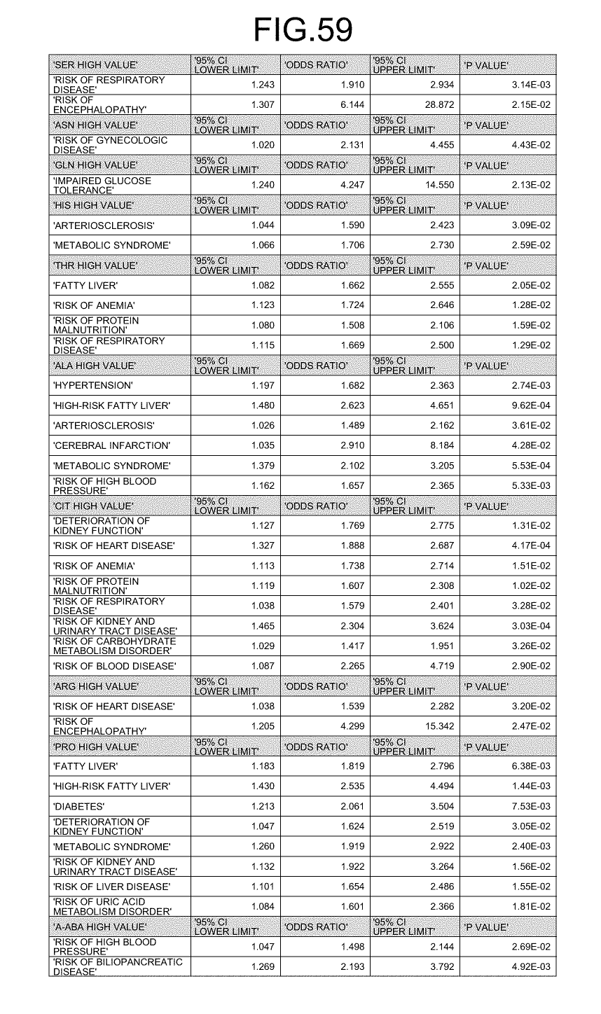

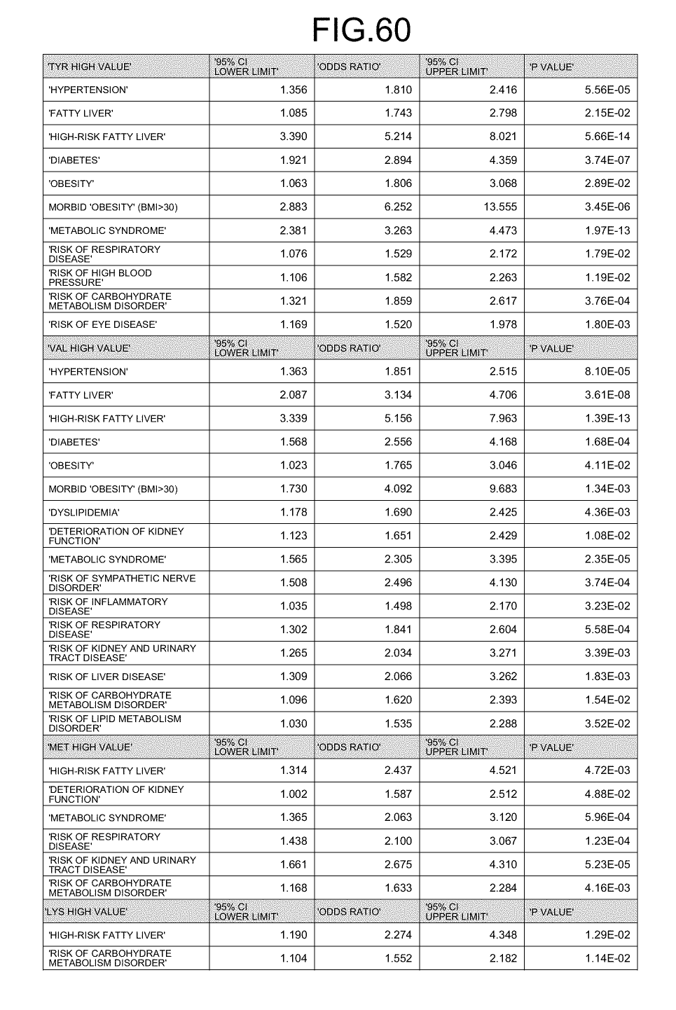

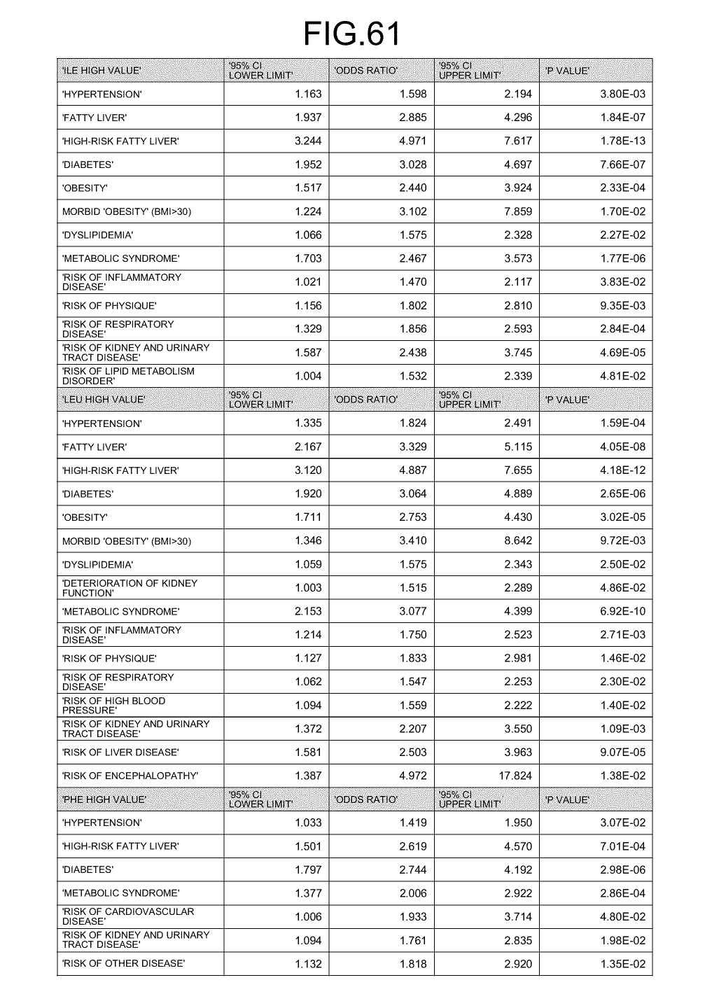

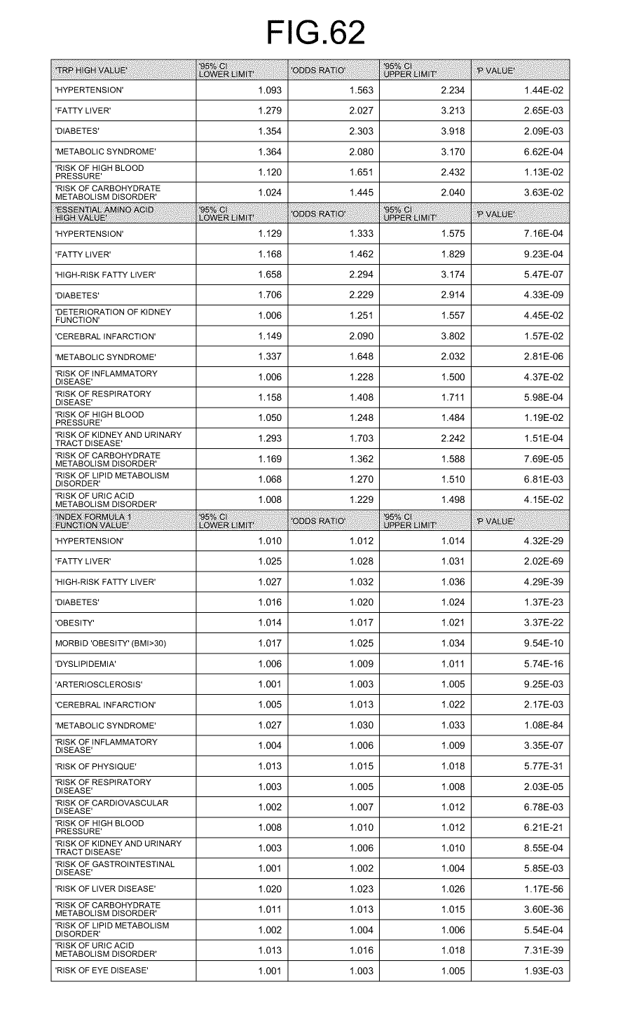

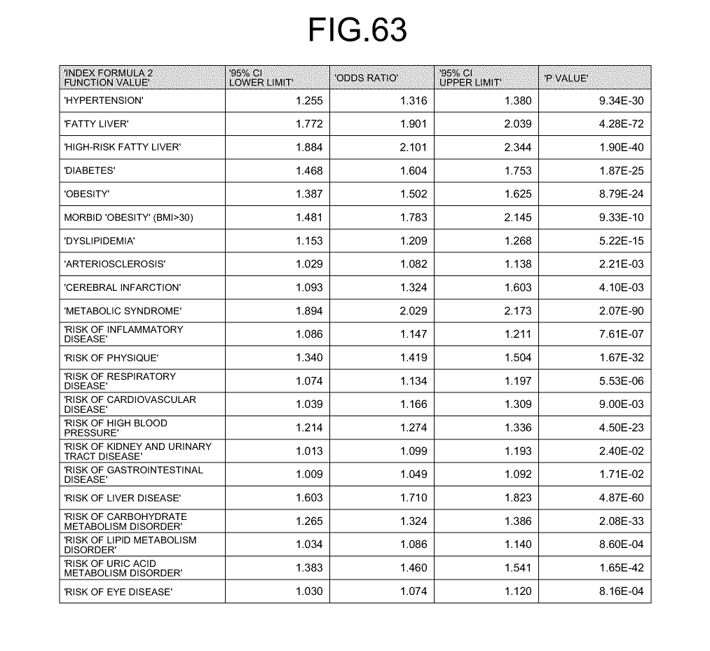

In FIG. 50 to FIG. 63, odds ratios when age as a background factor is adjusted, 95%-confidence interval lower limits and 95%-confidence interval upper limits thereof, and p values (p<0.05) of the odds ratios are each listed.

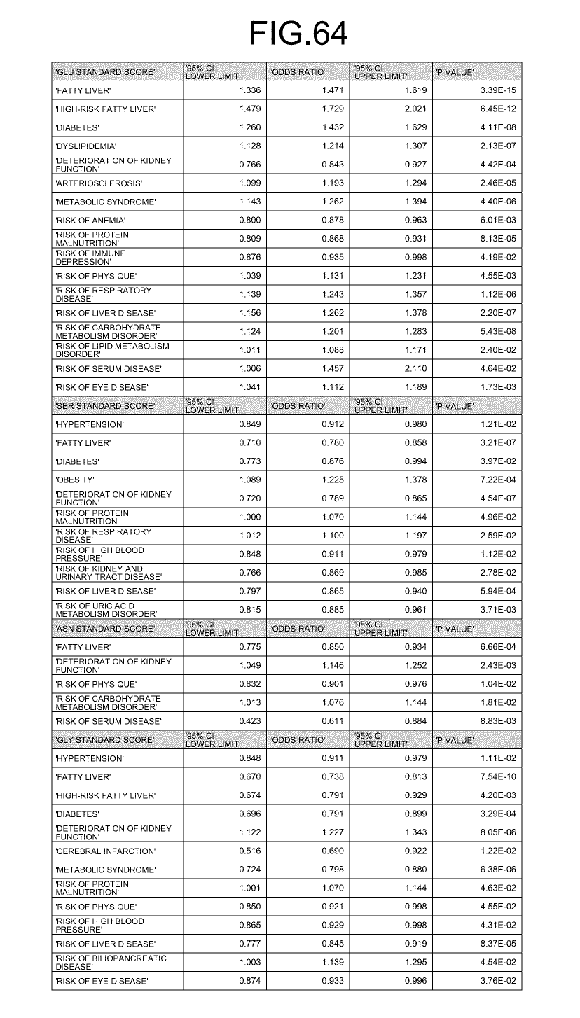

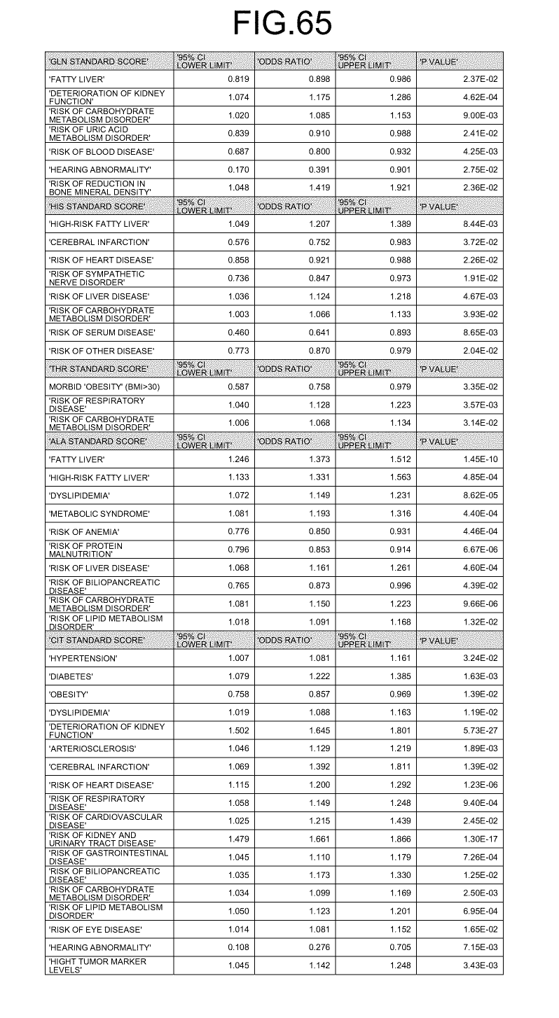

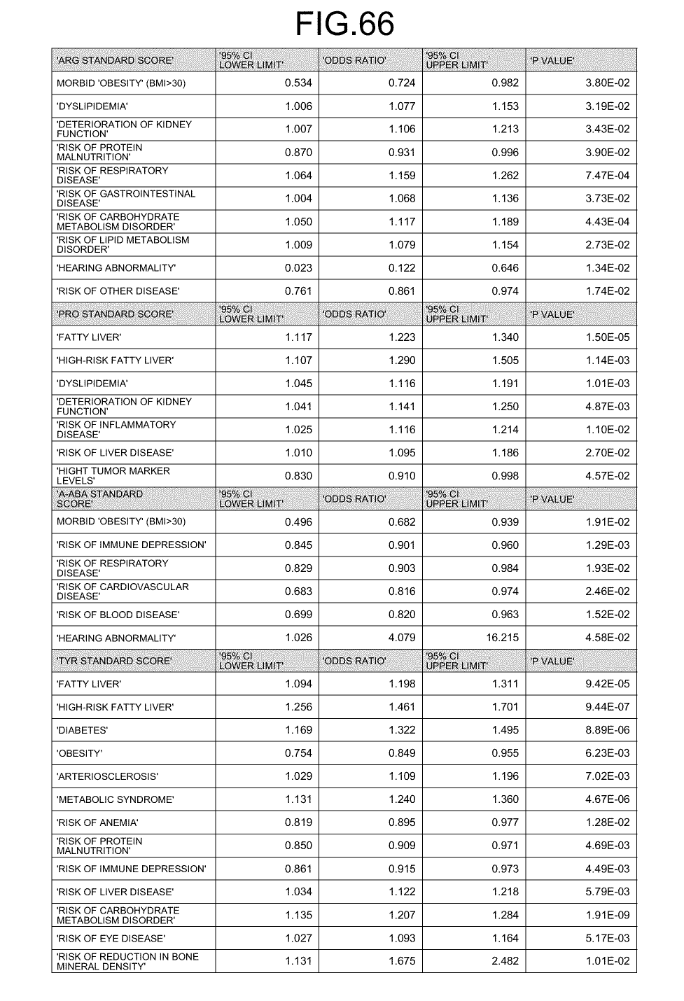

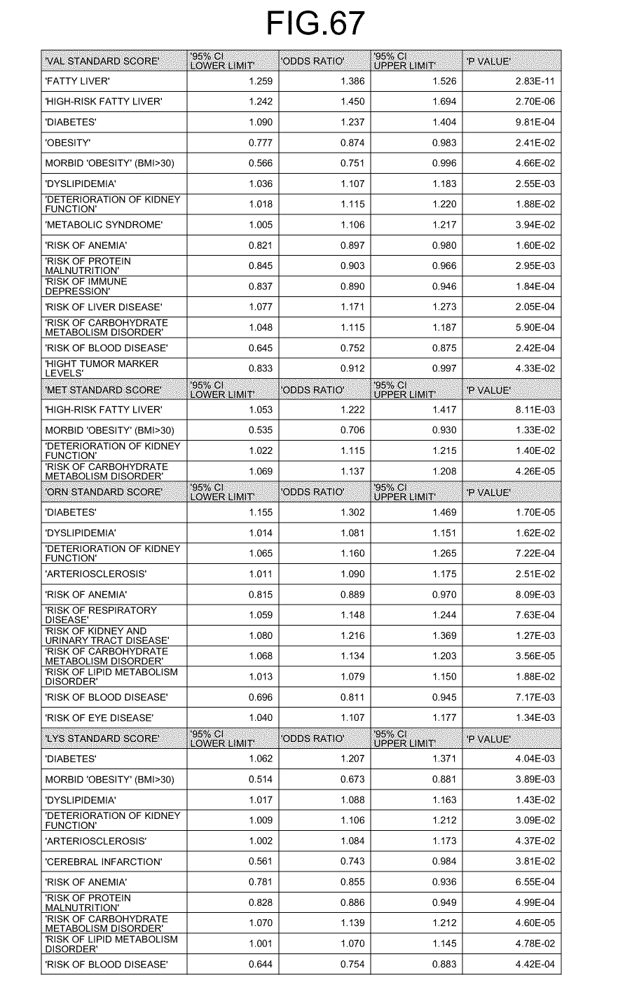

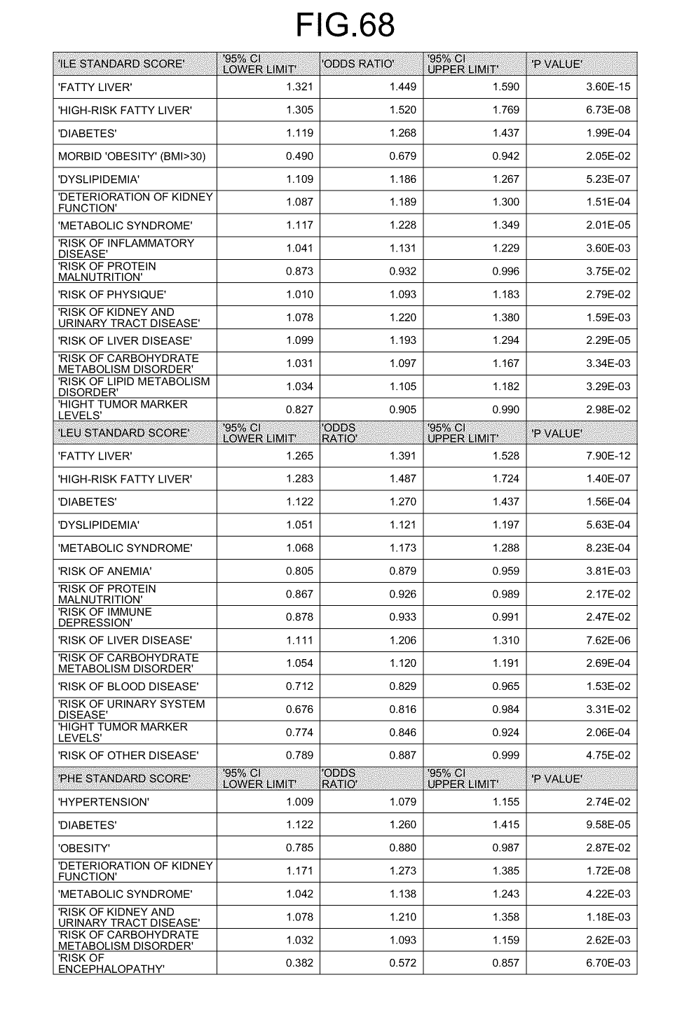

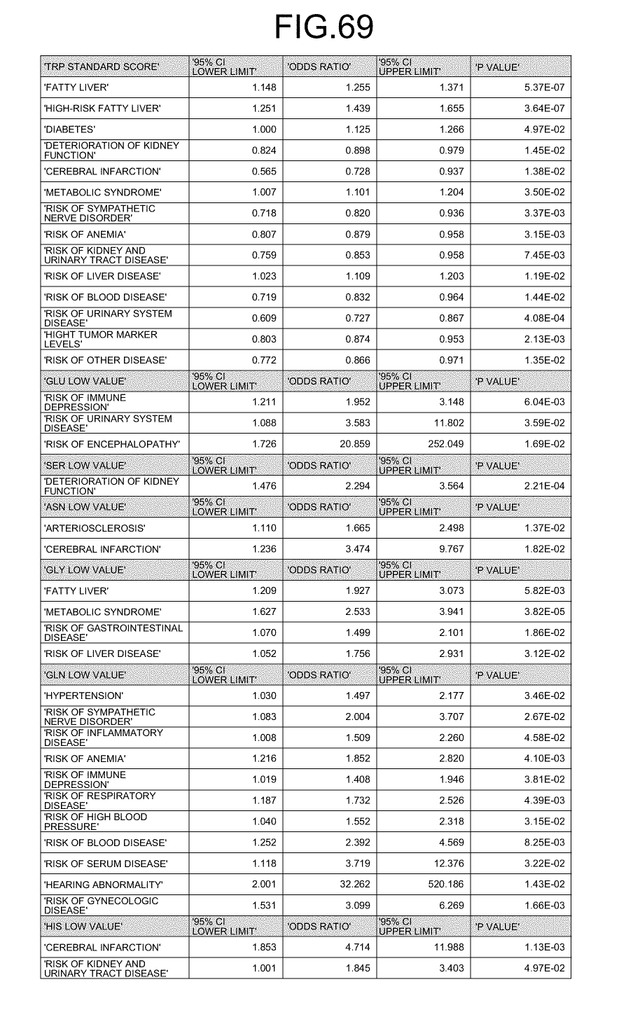

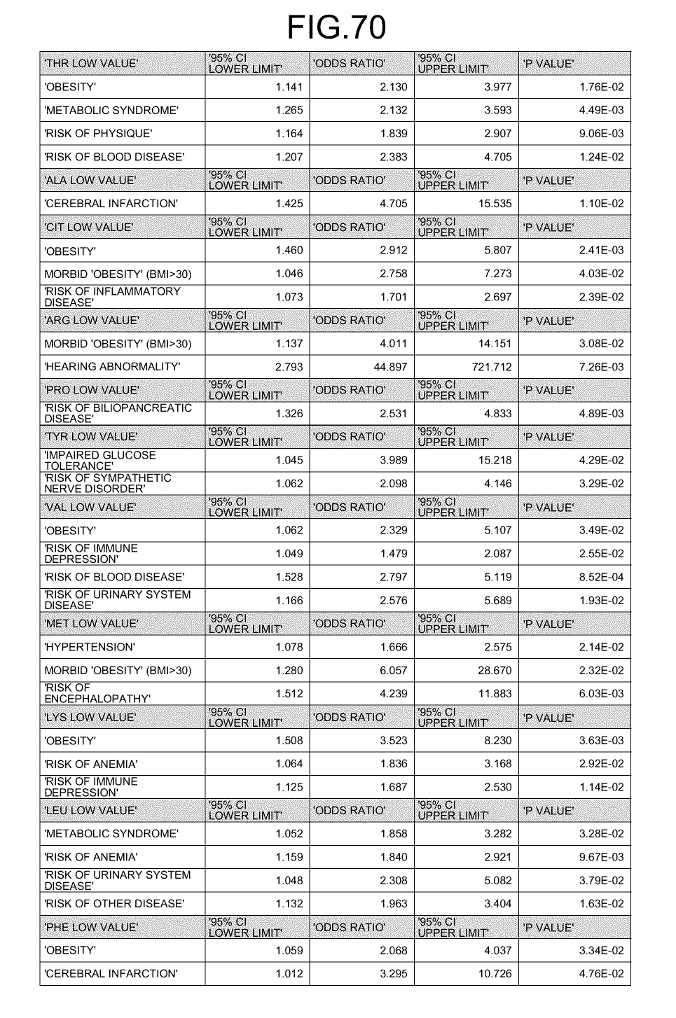

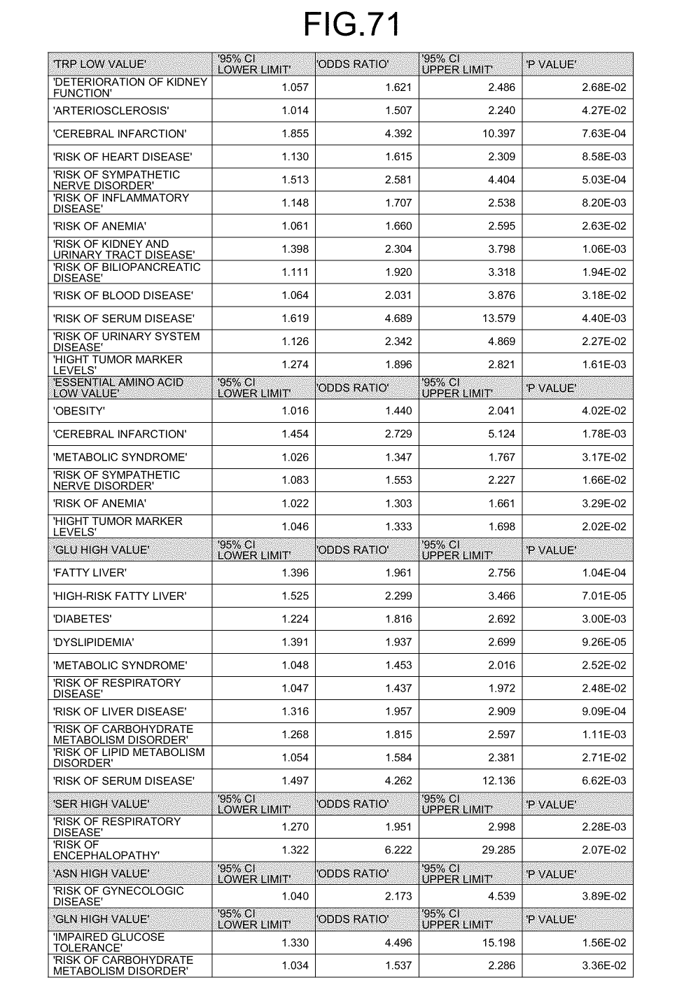

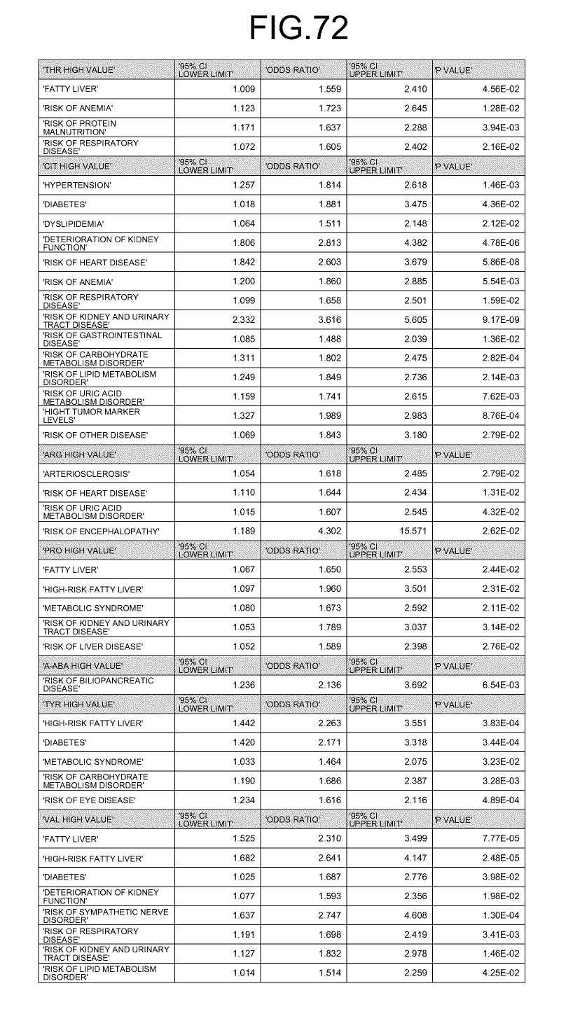

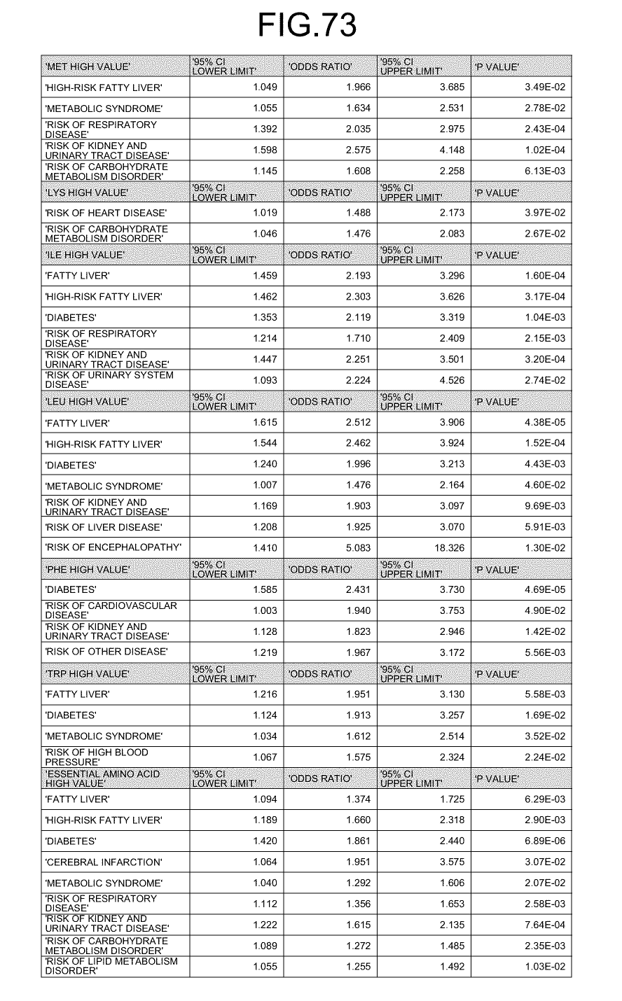

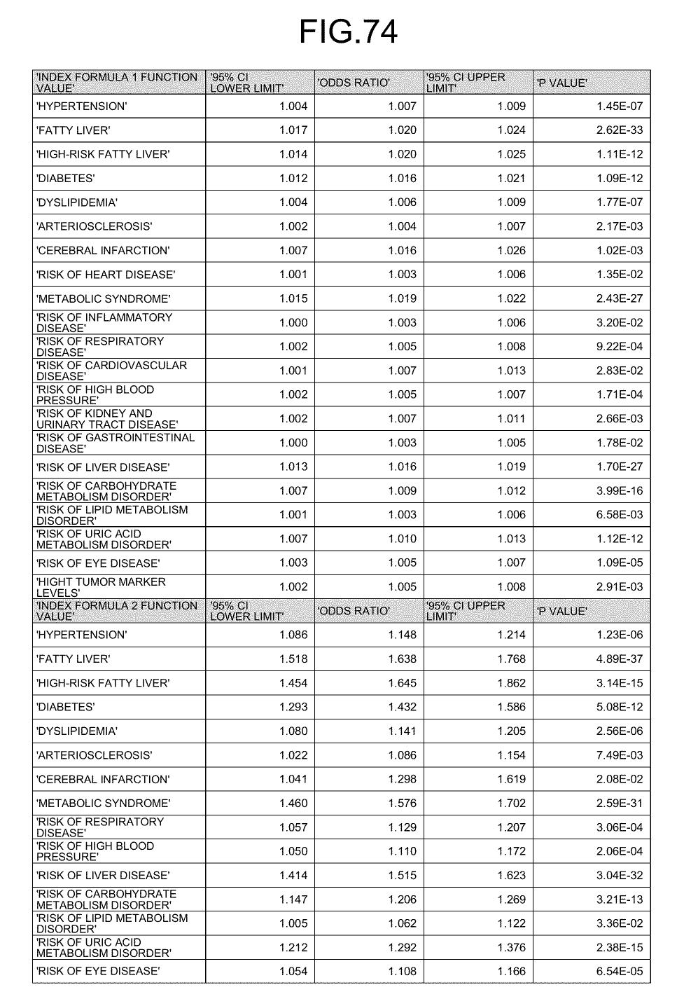

In FIG. 64 to FIG. 74, odds ratios when BMI as a background factor is adjusted, 95%-confidence interval lower limits and 95%-confidence interval upper limits thereof, and p values (p<0.05) of the odds ratios are each listed.

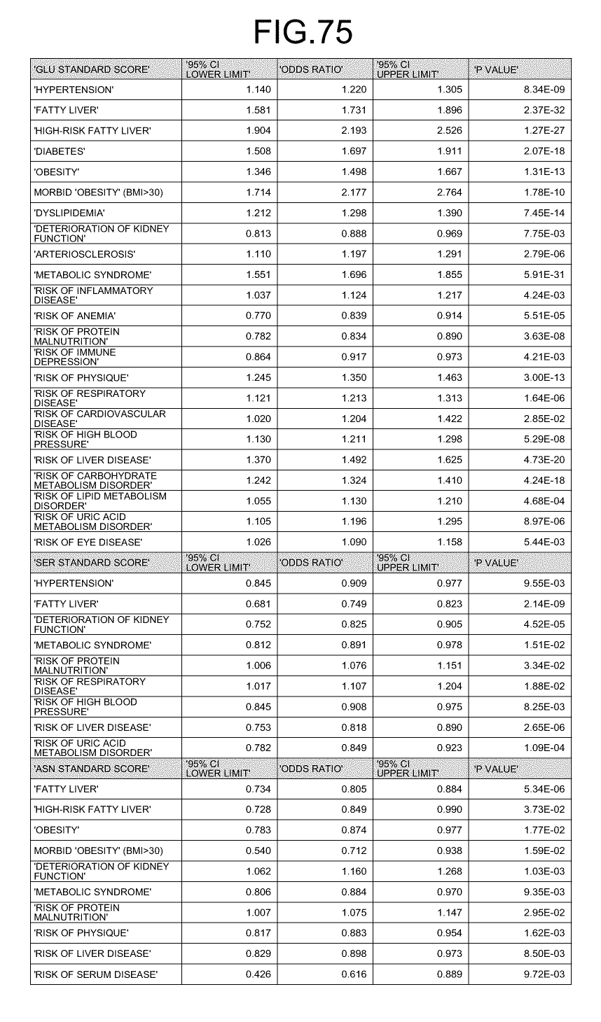

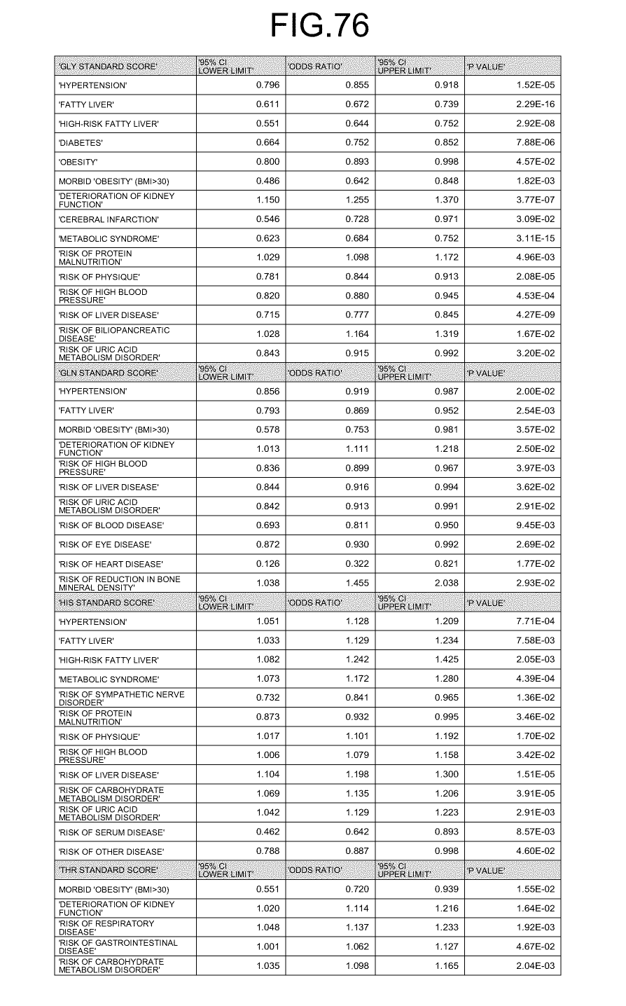

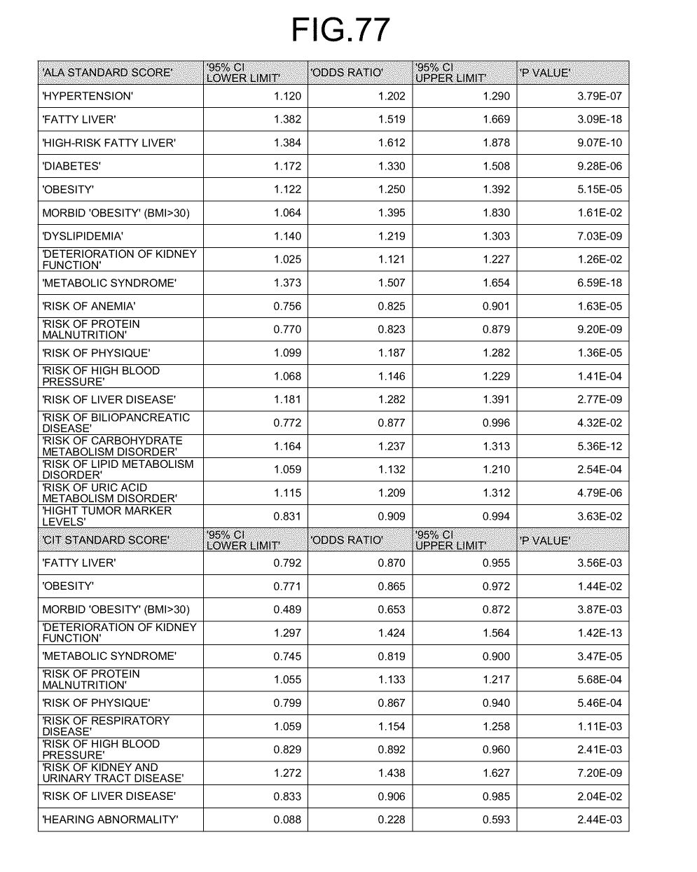

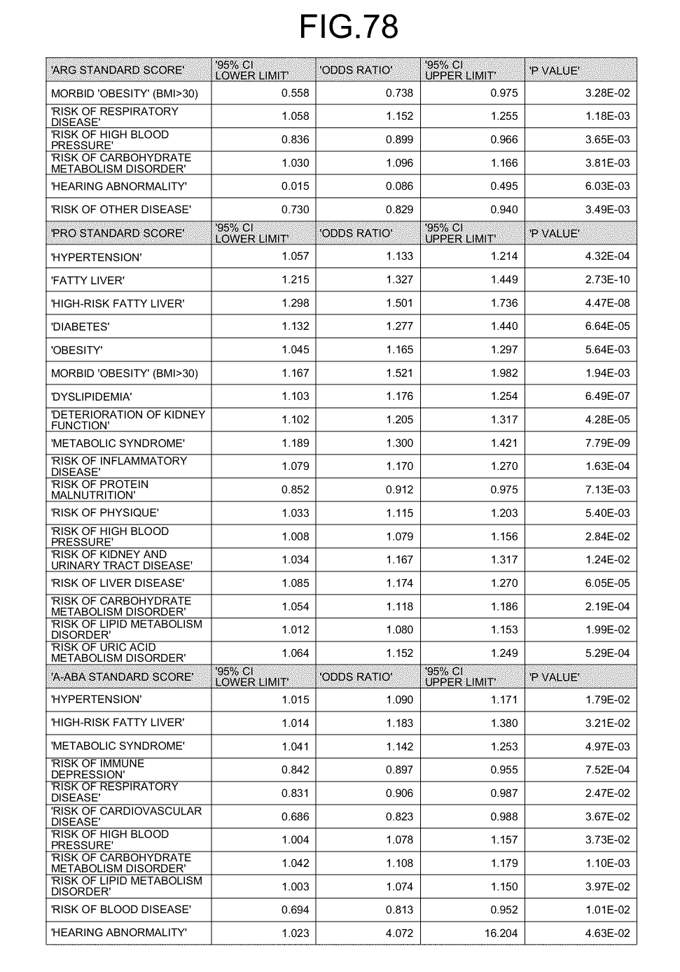

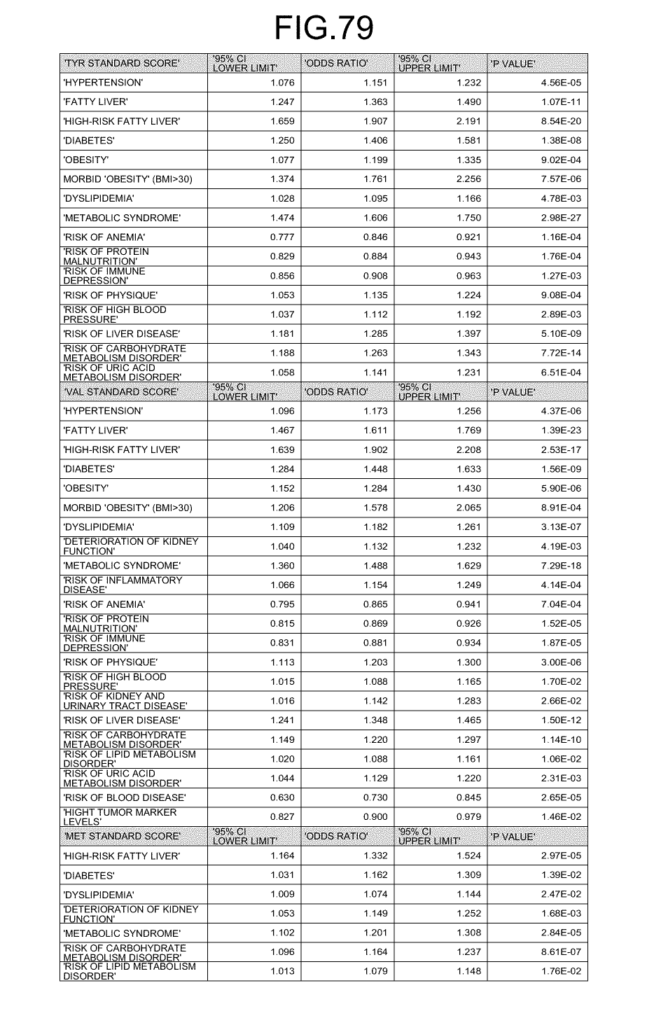

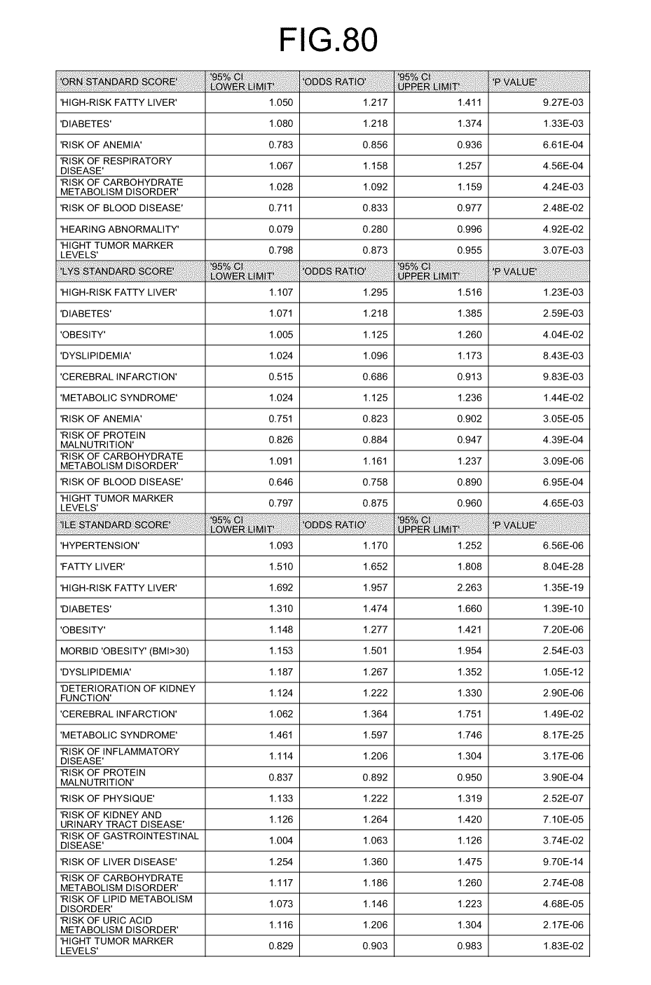

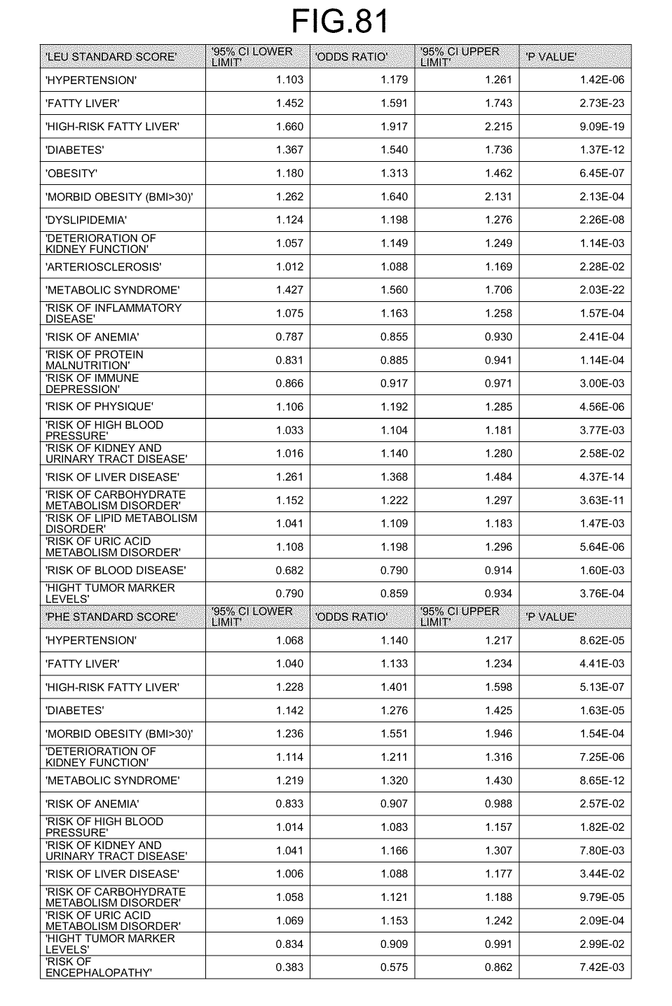

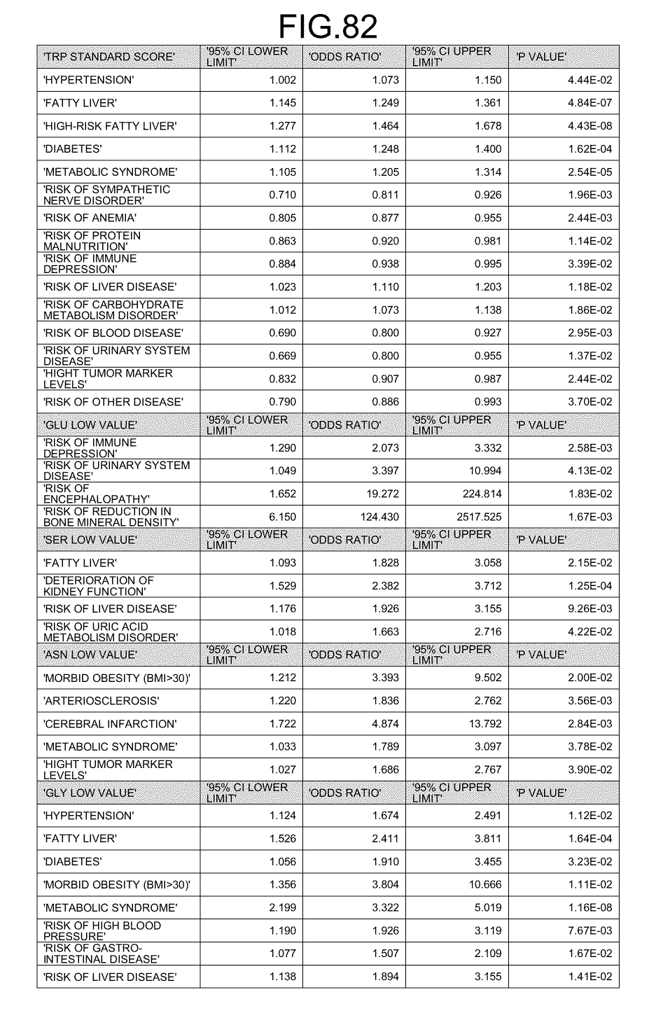

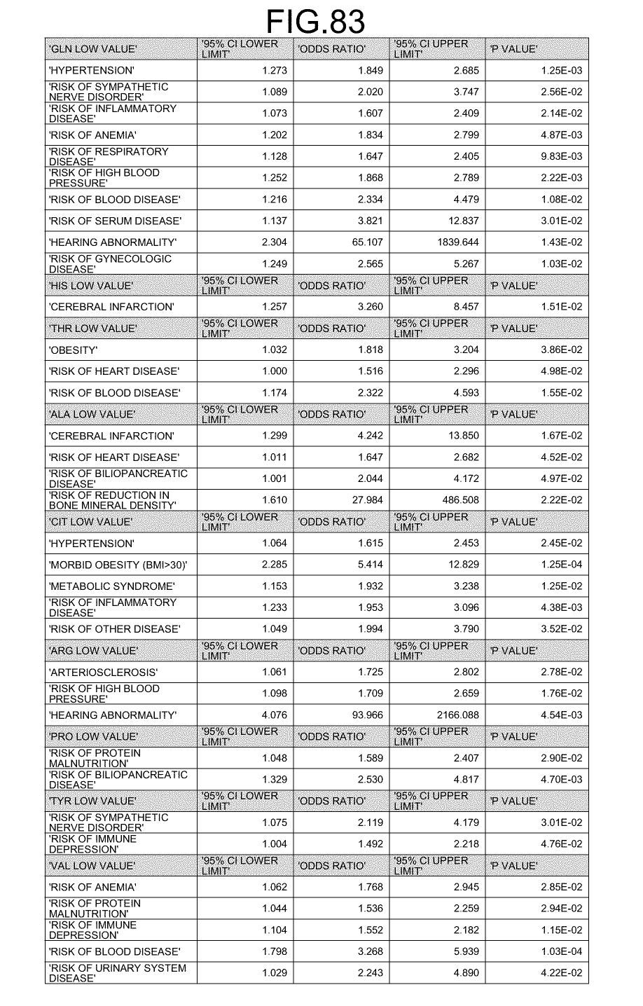

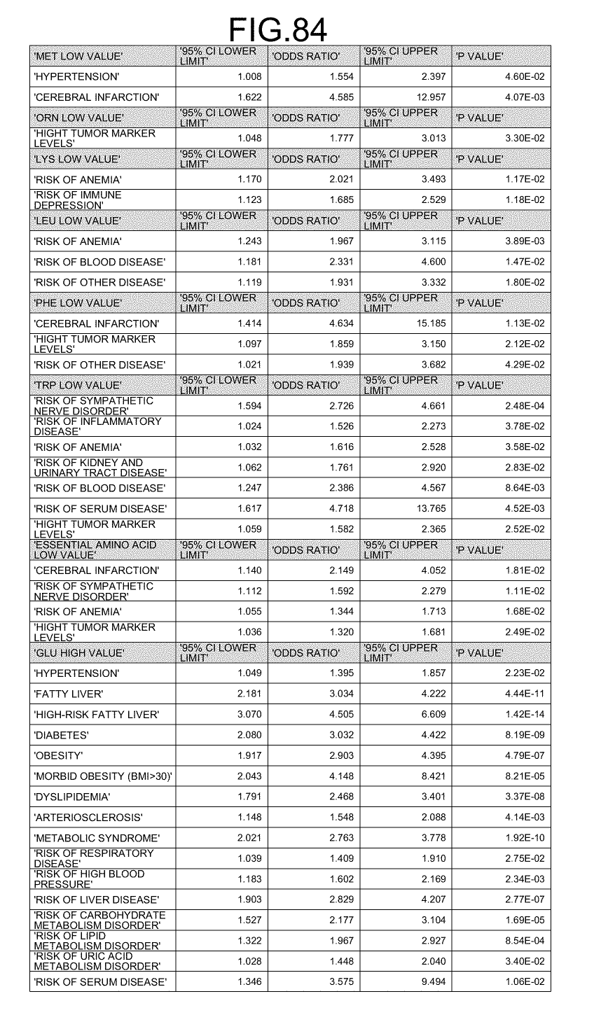

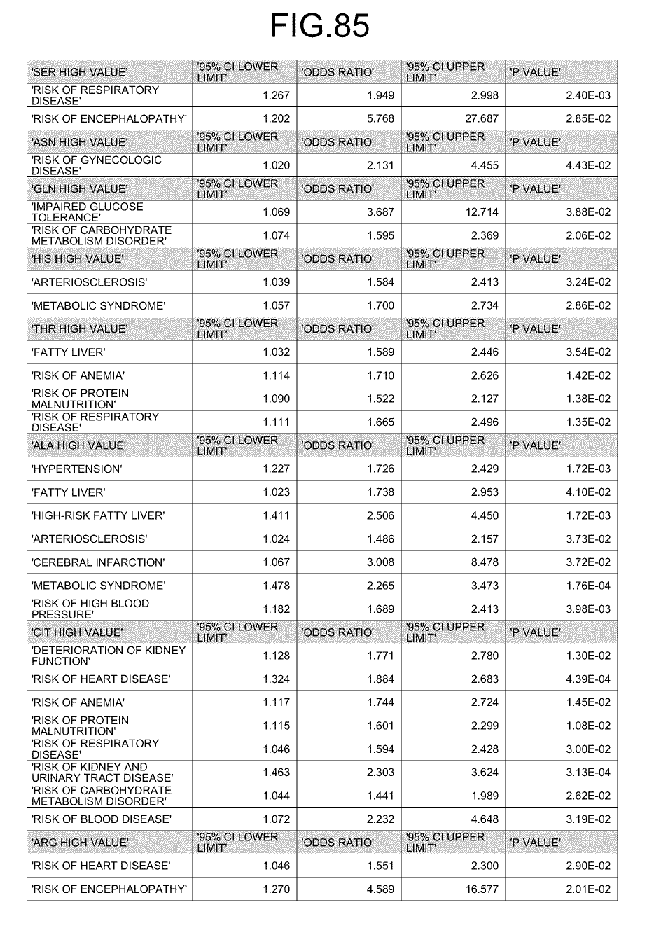

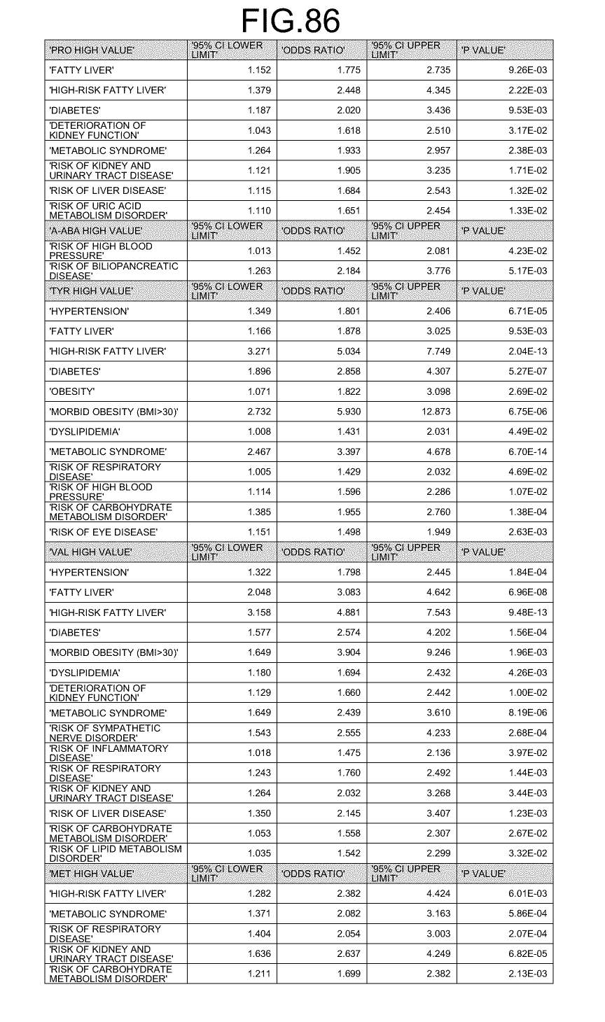

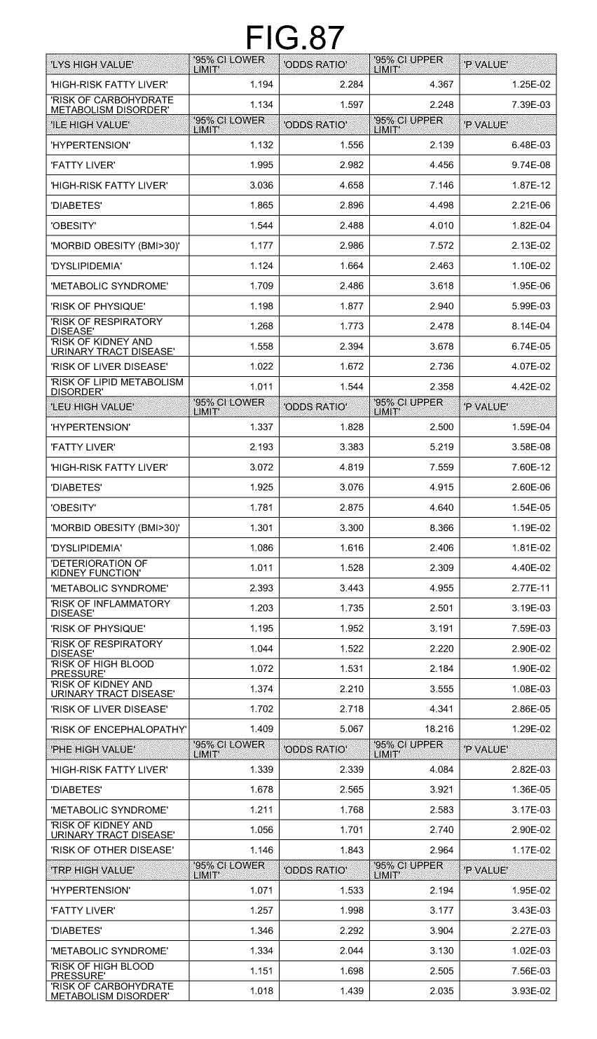

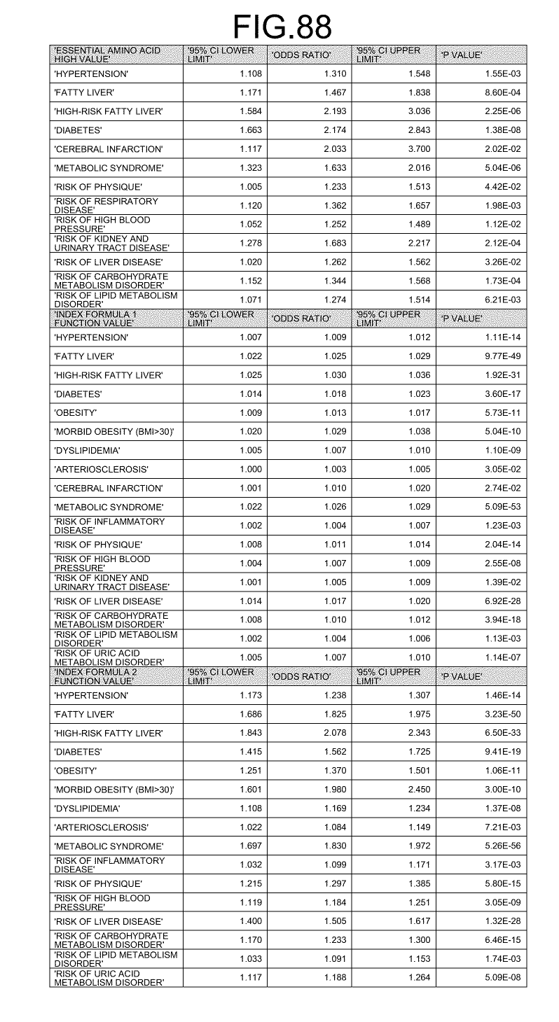

In FIG. 75 to FIG. 88, odds ratios when gender and age as background factors are adjusted, 95%-confidence interval lower limits and 95%-confidence interval upper limits thereof, and p values (p<0.05) of the odds ratios are each listed.

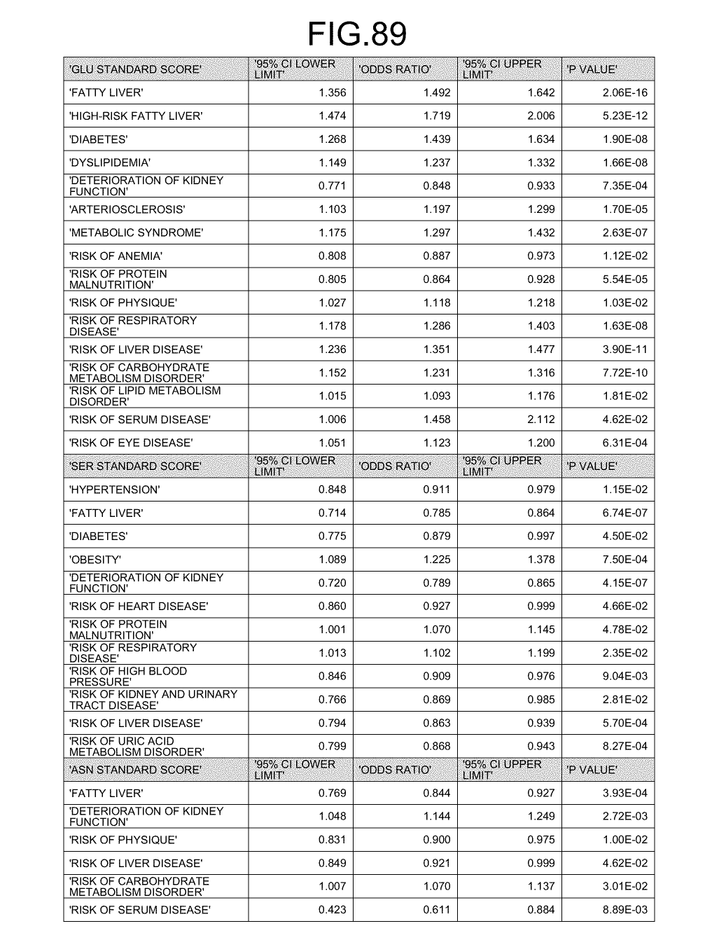

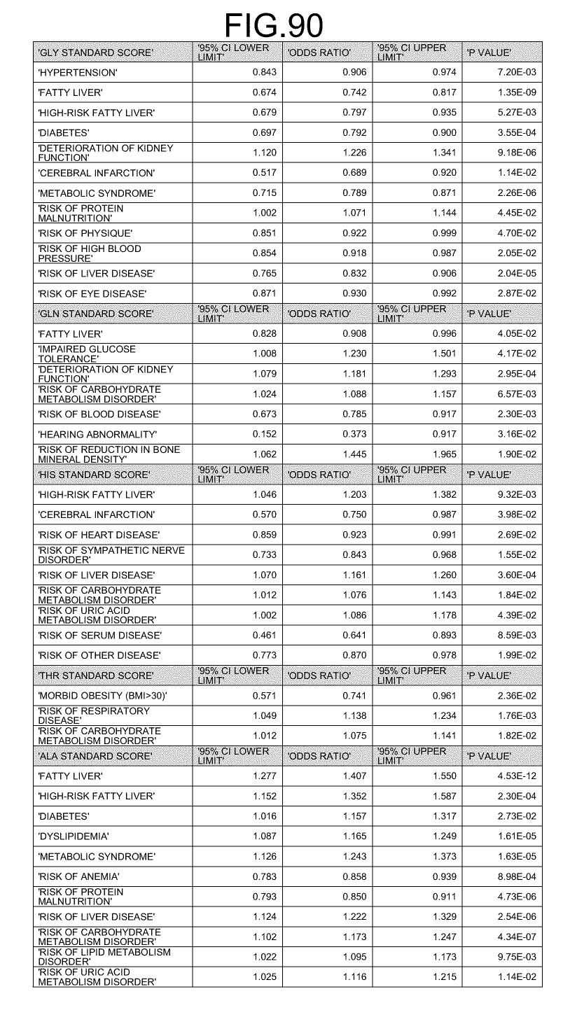

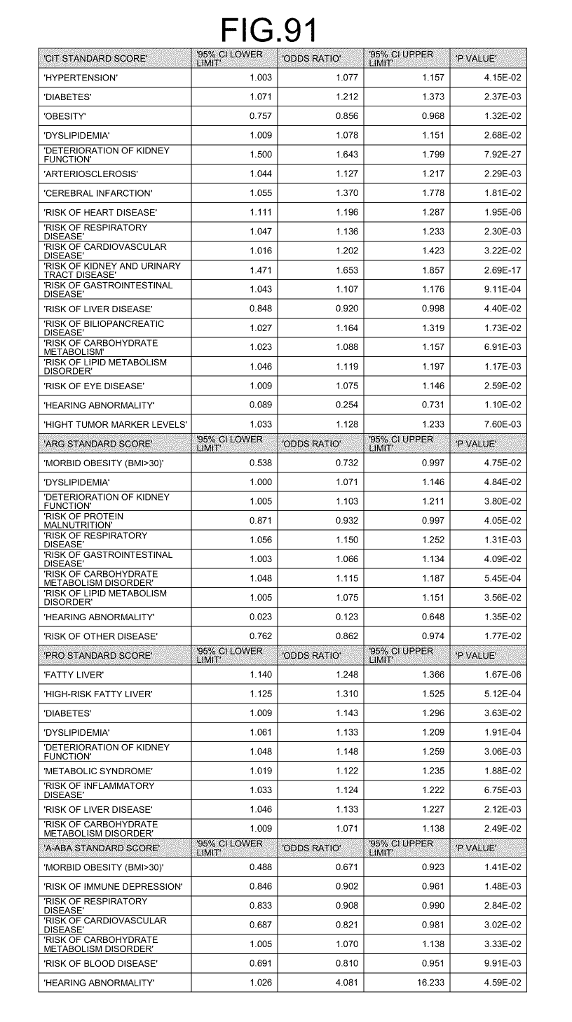

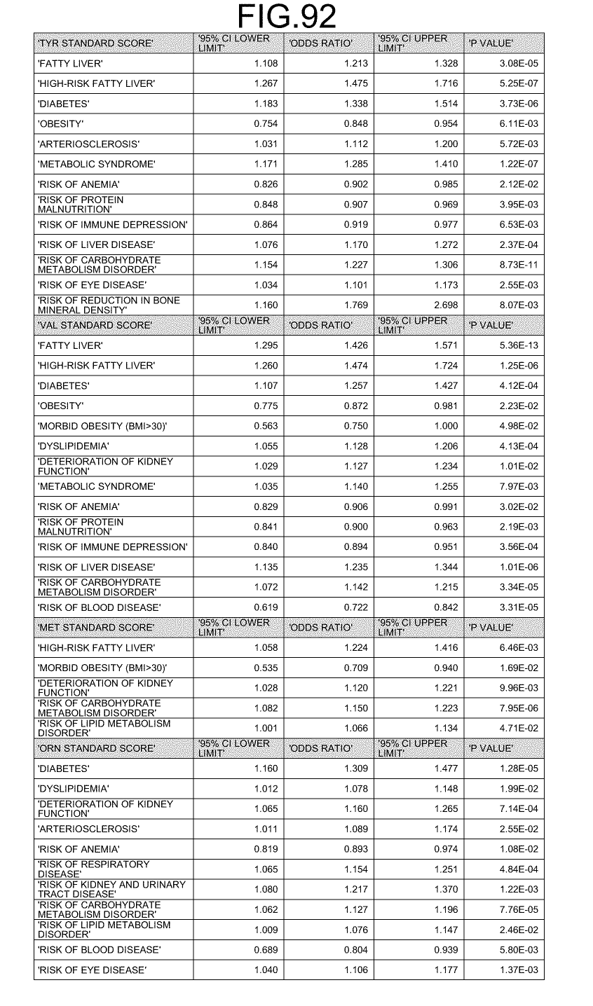

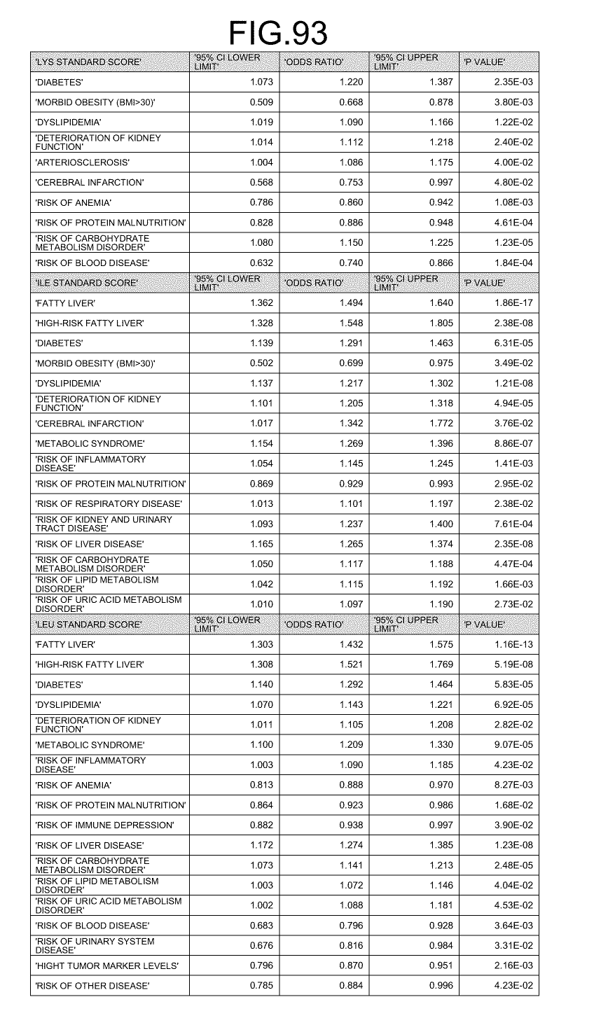

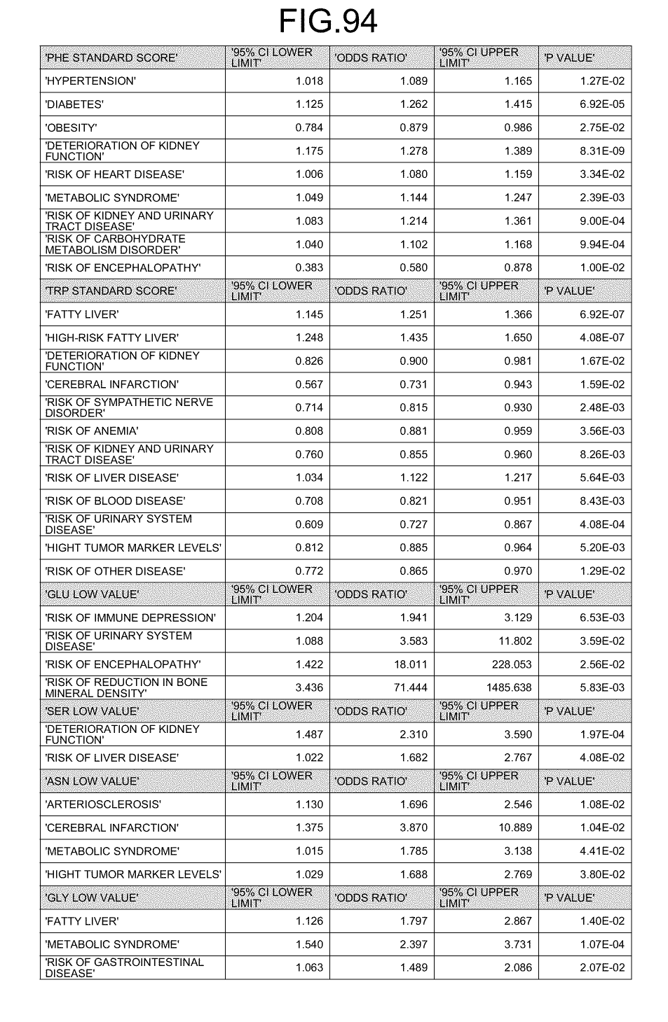

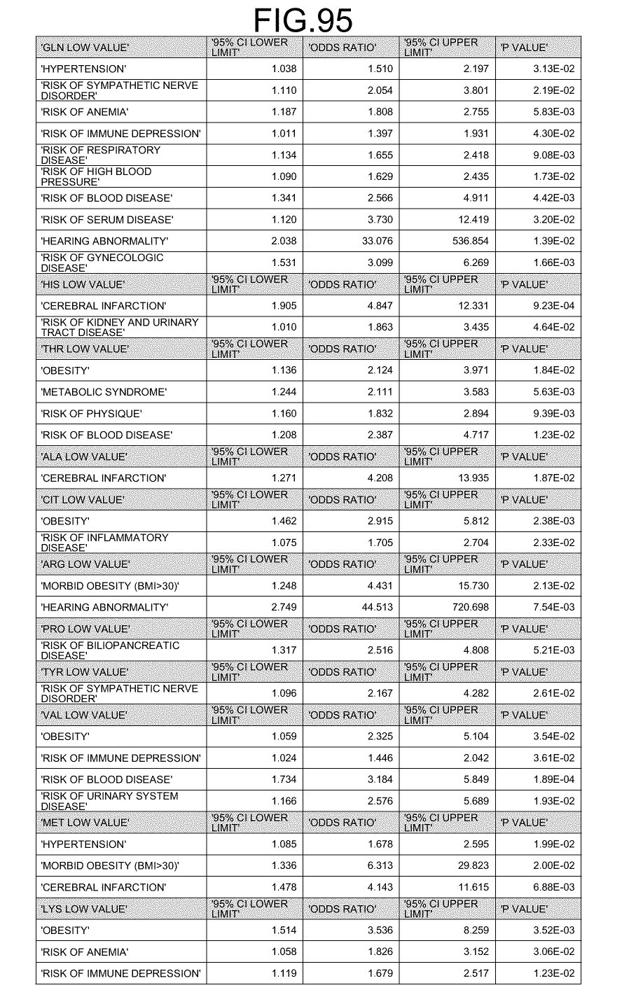

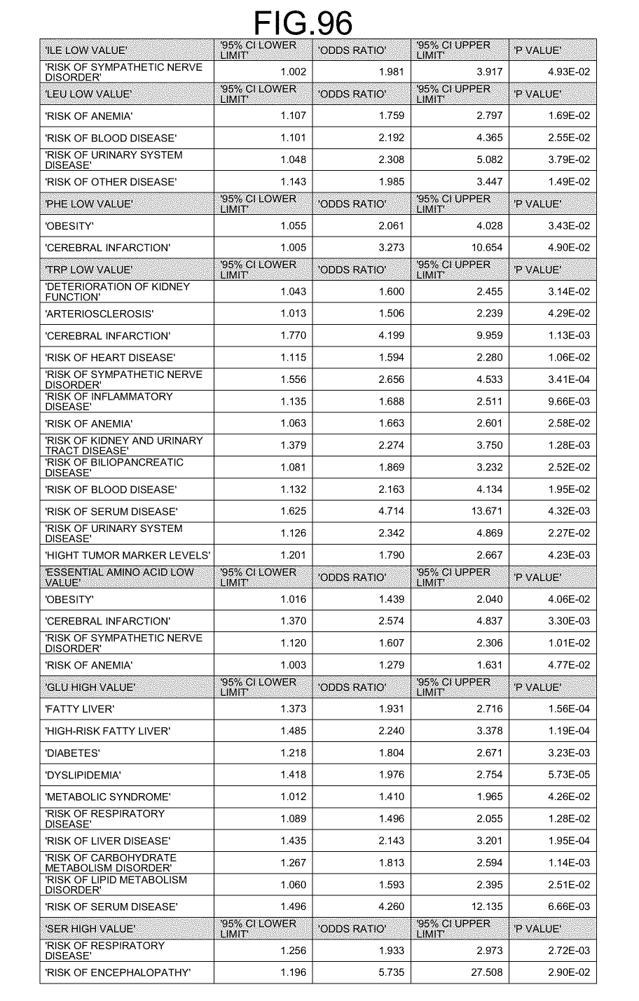

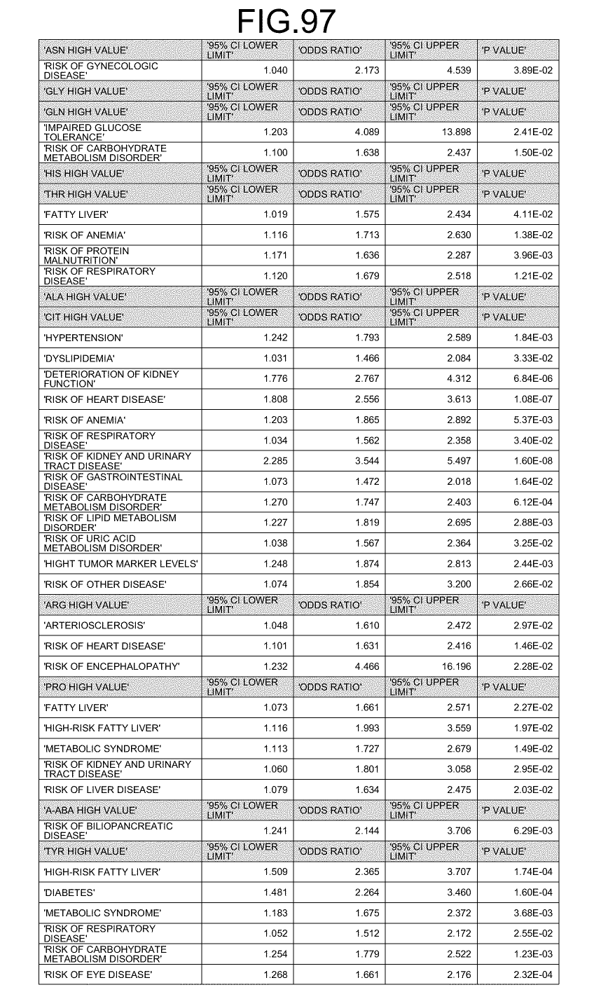

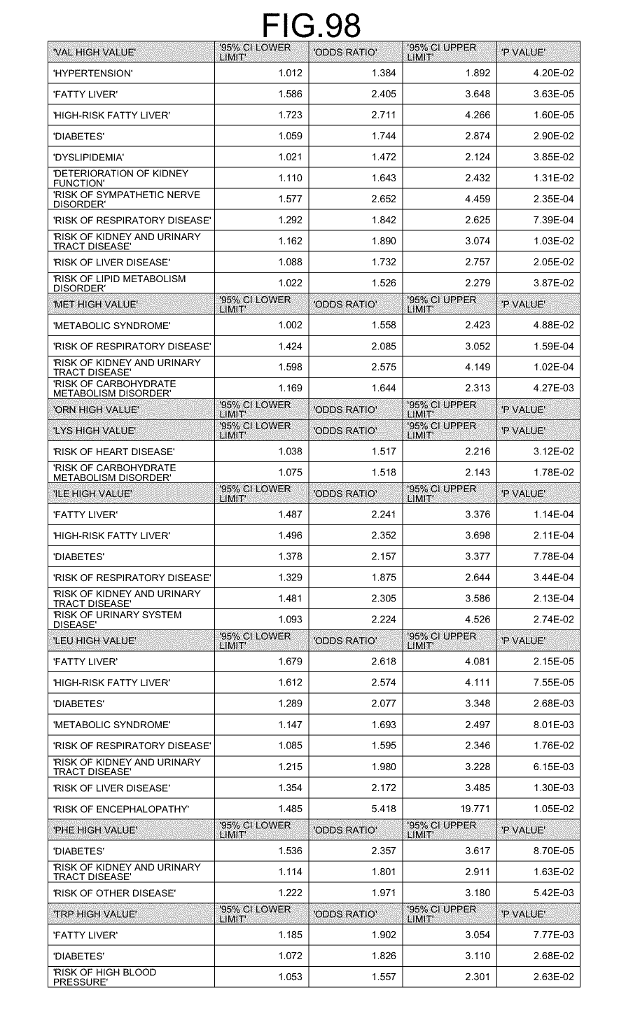

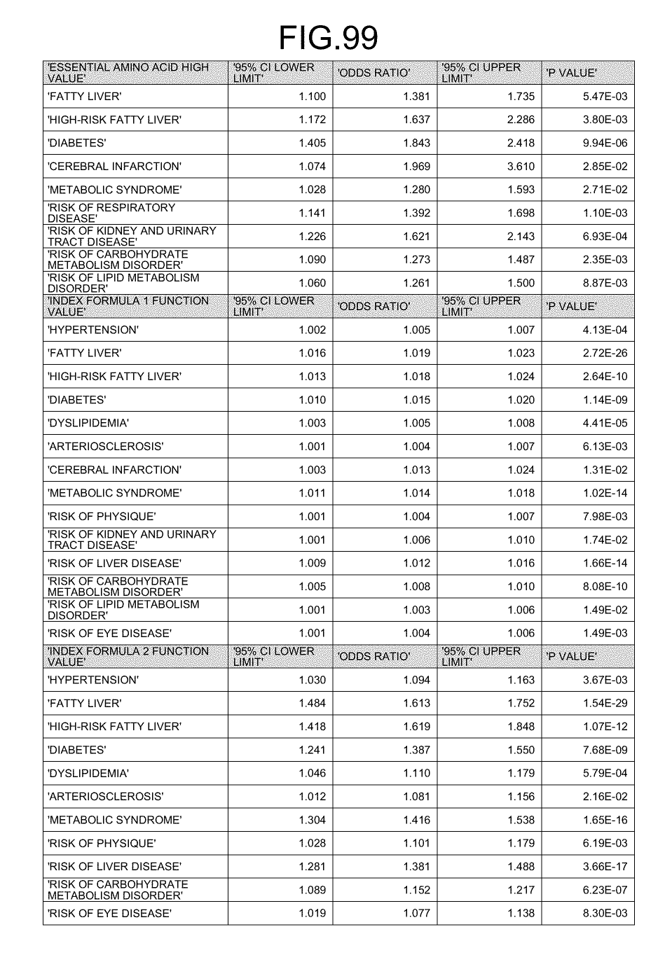

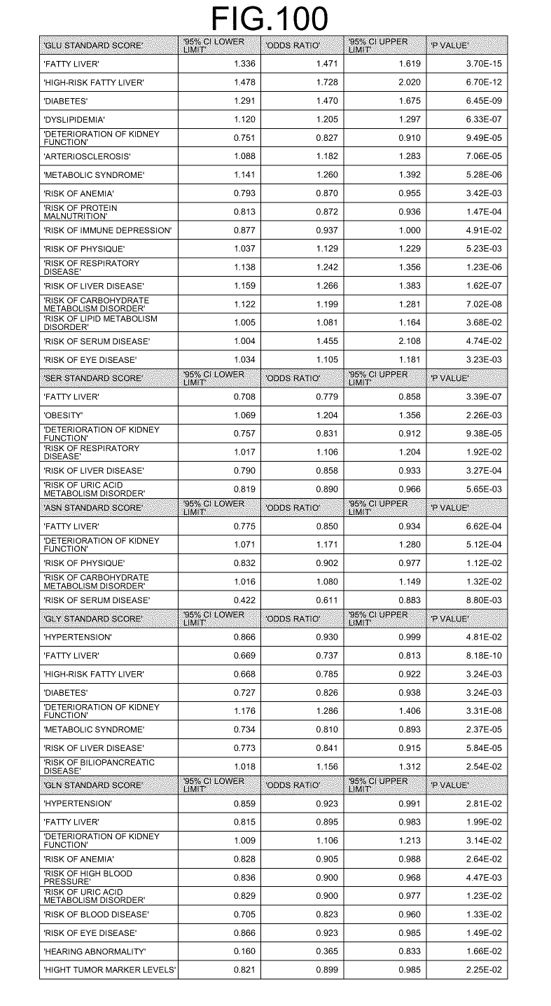

In FIG. 89 to FIG. 99, odds ratios when gender and BMI as background factors are adjusted, 95%-confidence interval lower limits and 95%-confidence interval upper limits thereof, and p values (p<0.05) of the odds ratios are each listed.

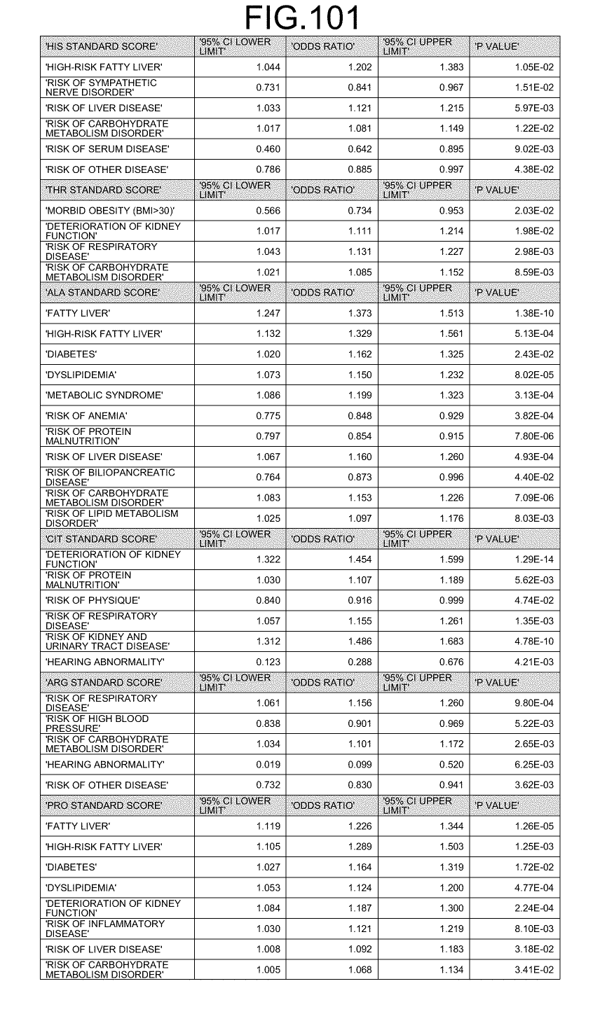

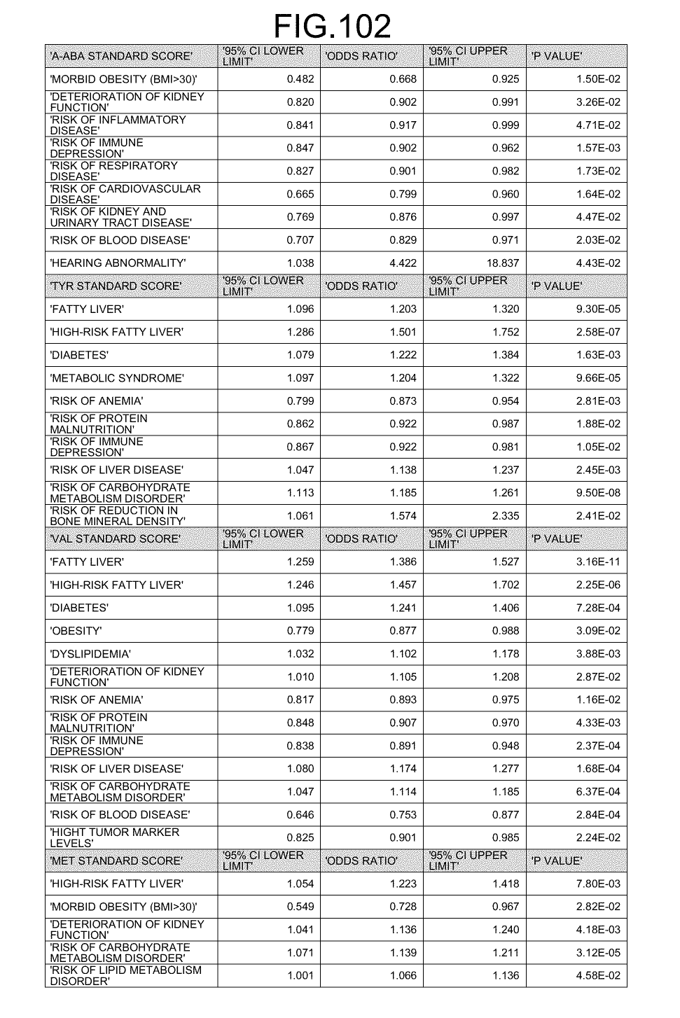

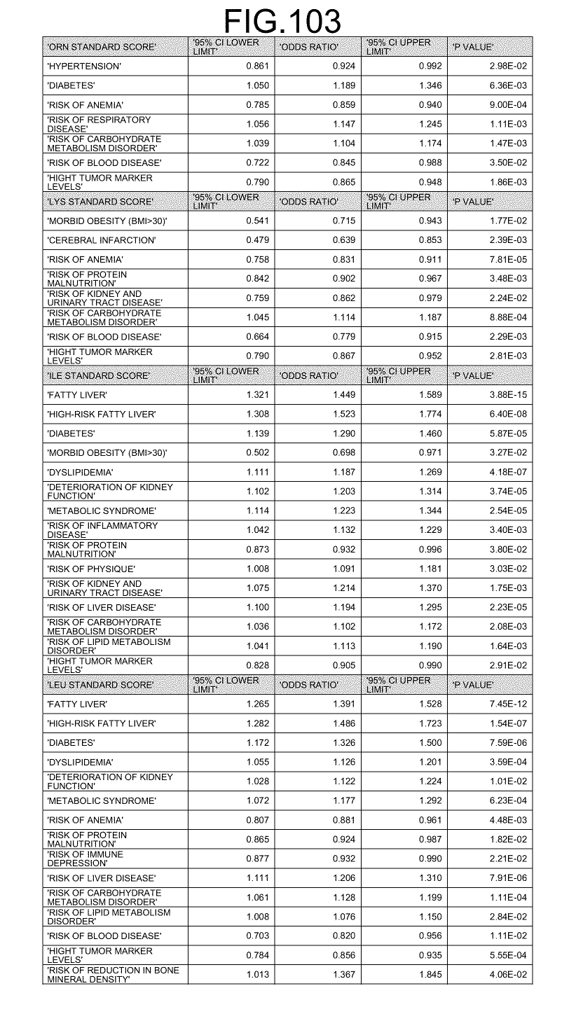

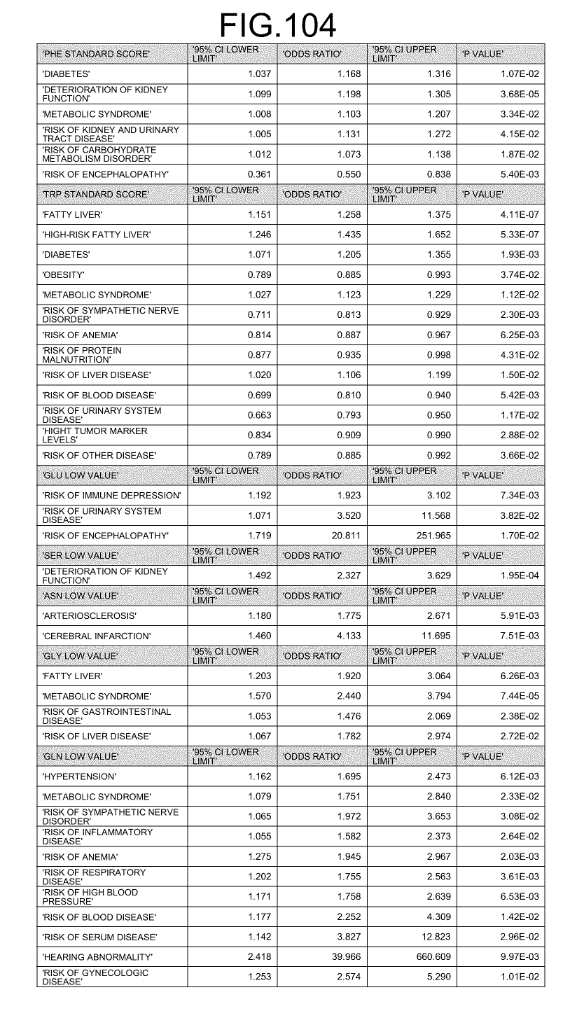

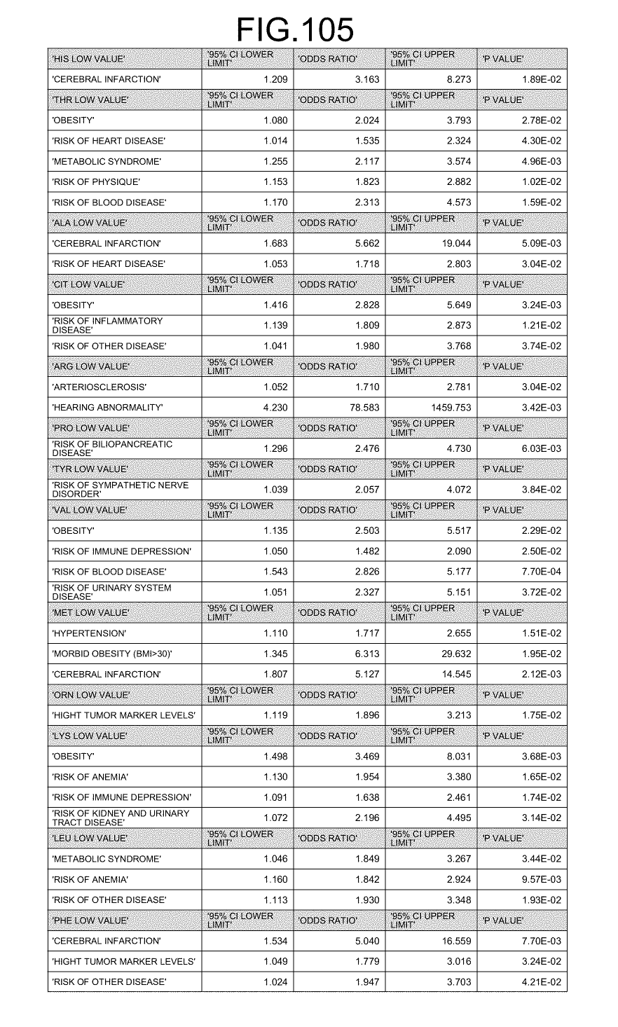

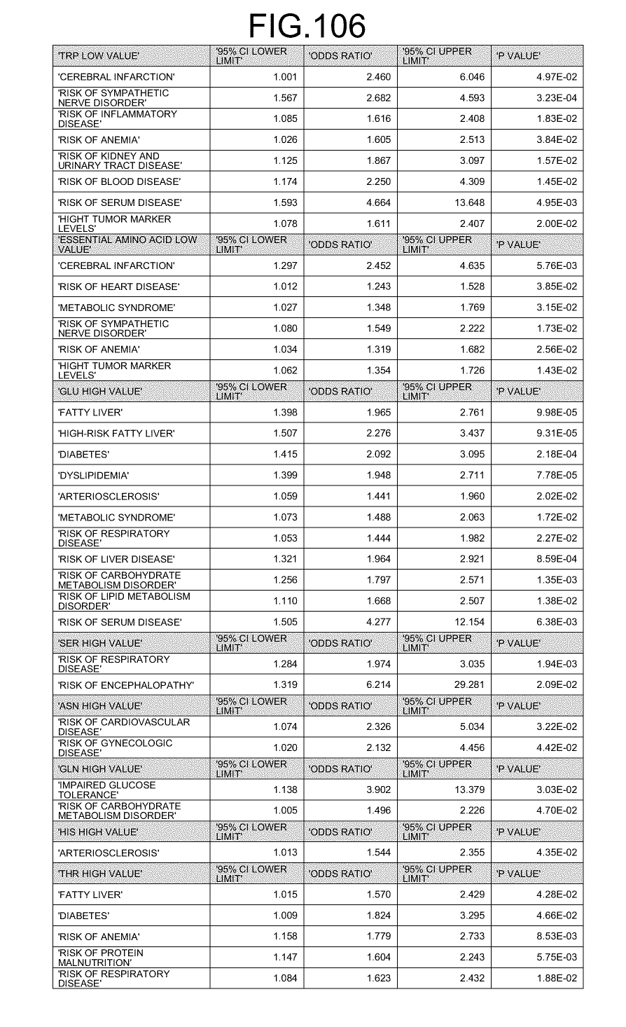

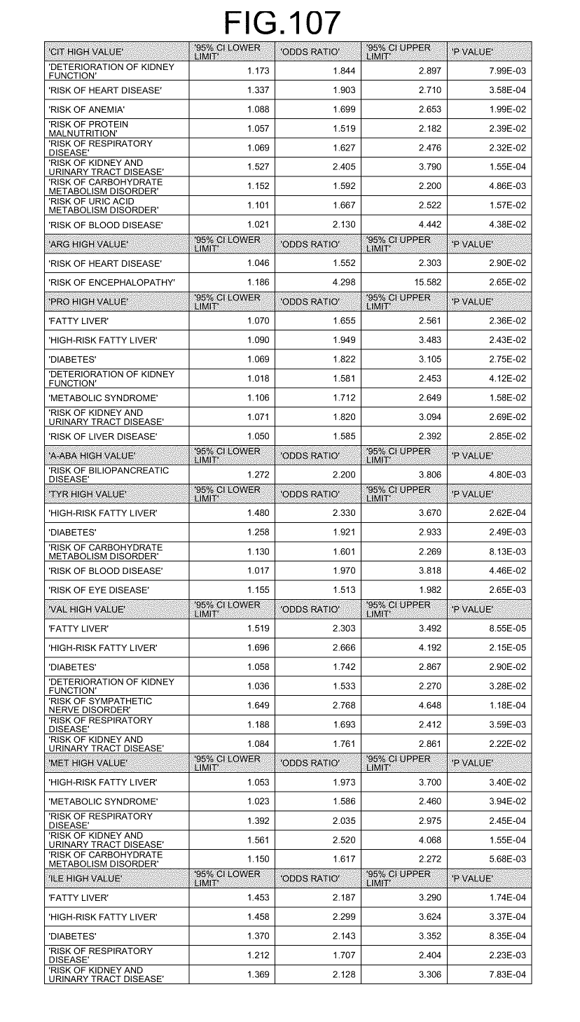

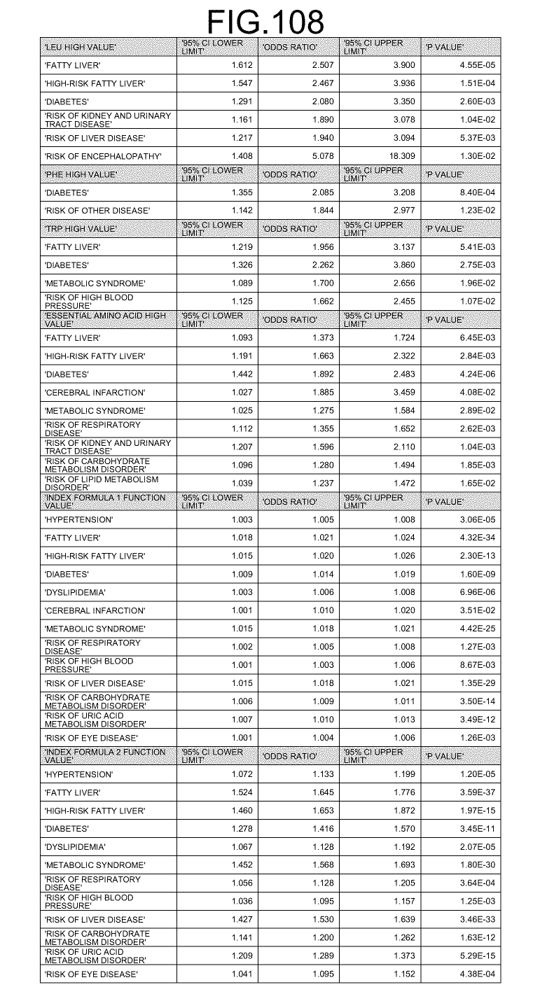

In FIG. 100 to FIG. 108, odds ratios when age and BMI as background factors are adjusted, 95%-confidence interval lower limits and 95%-confidence interval upper limits thereof, and p values (p<0.05) of the odds ratios are each listed.

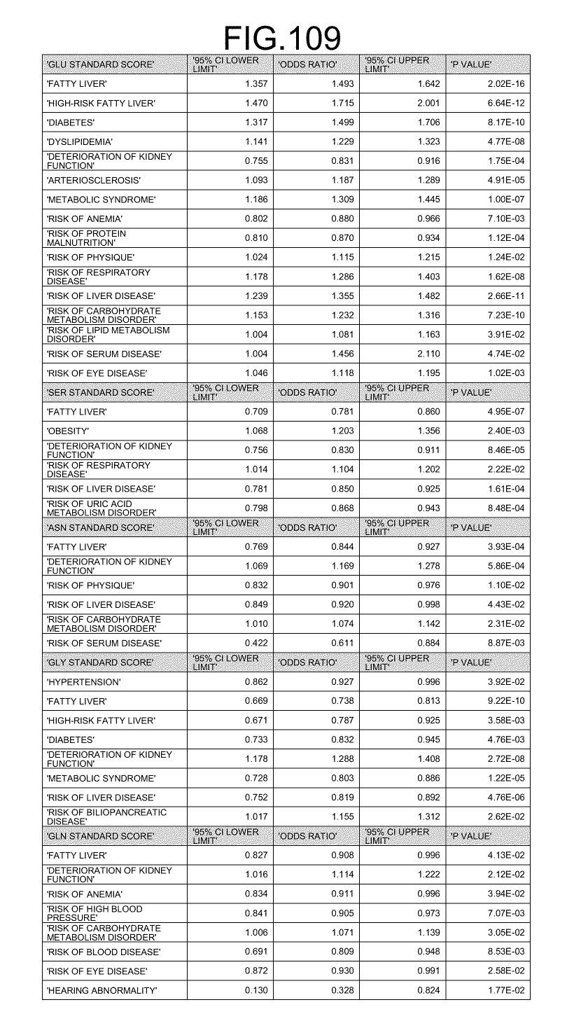

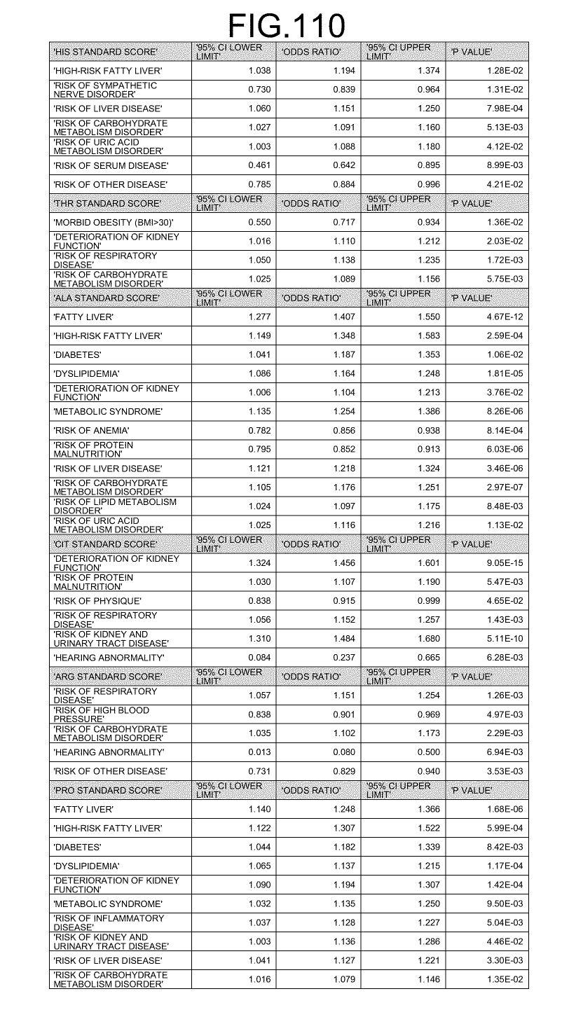

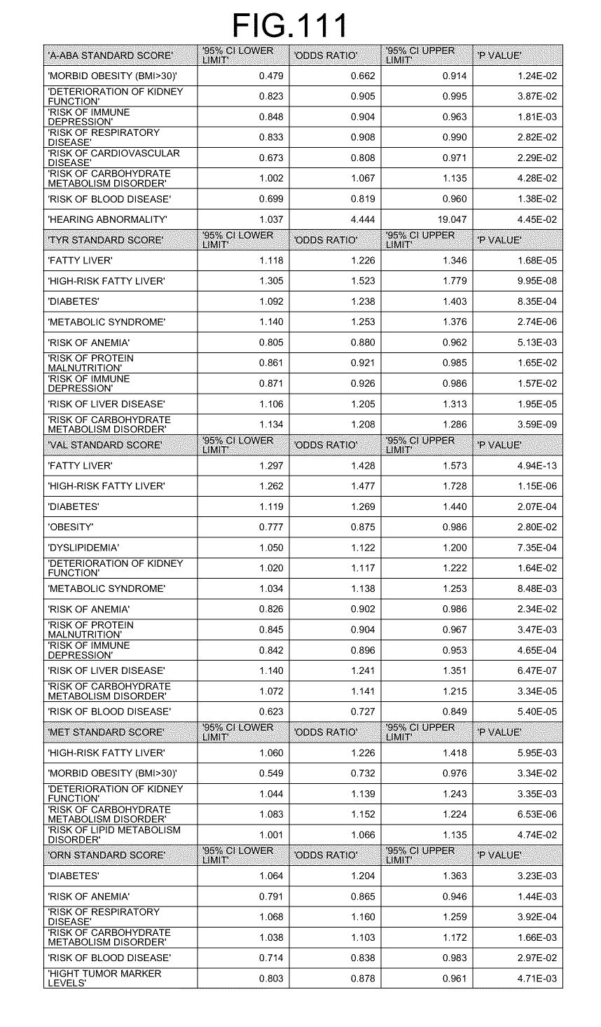

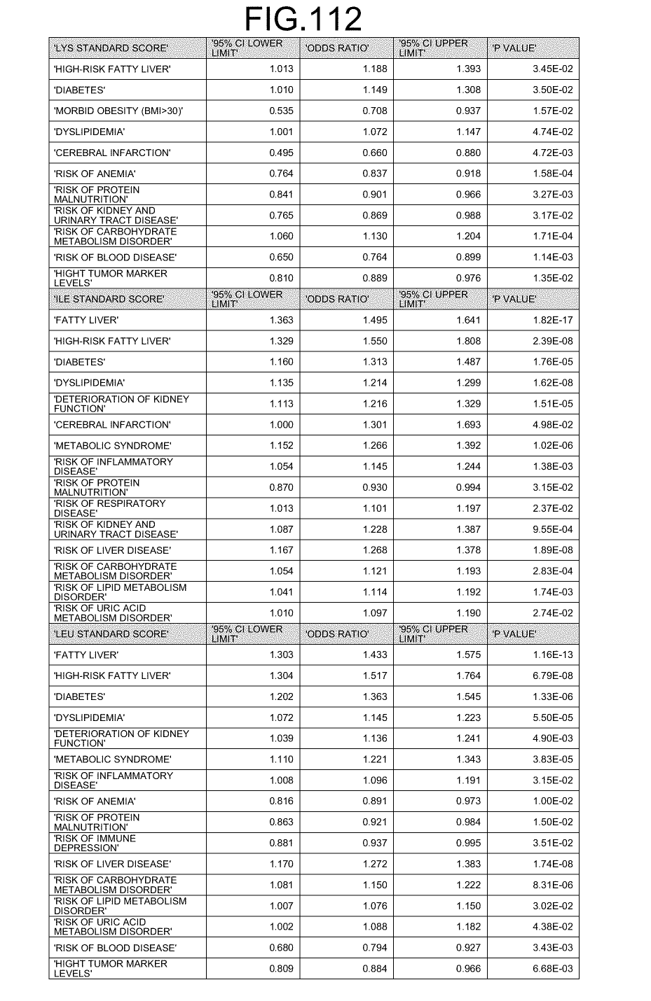

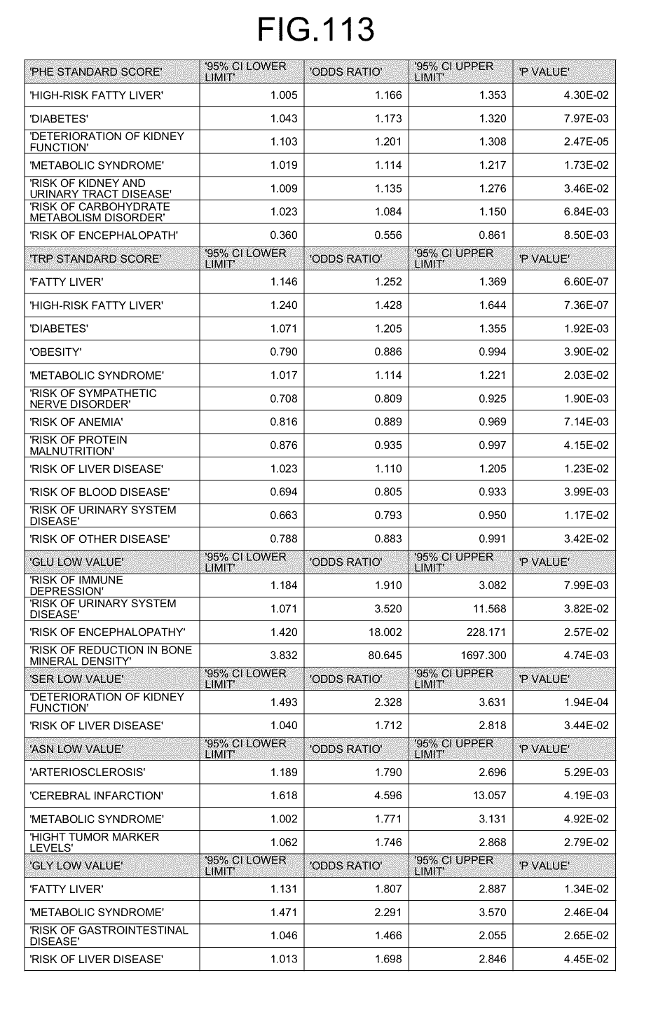

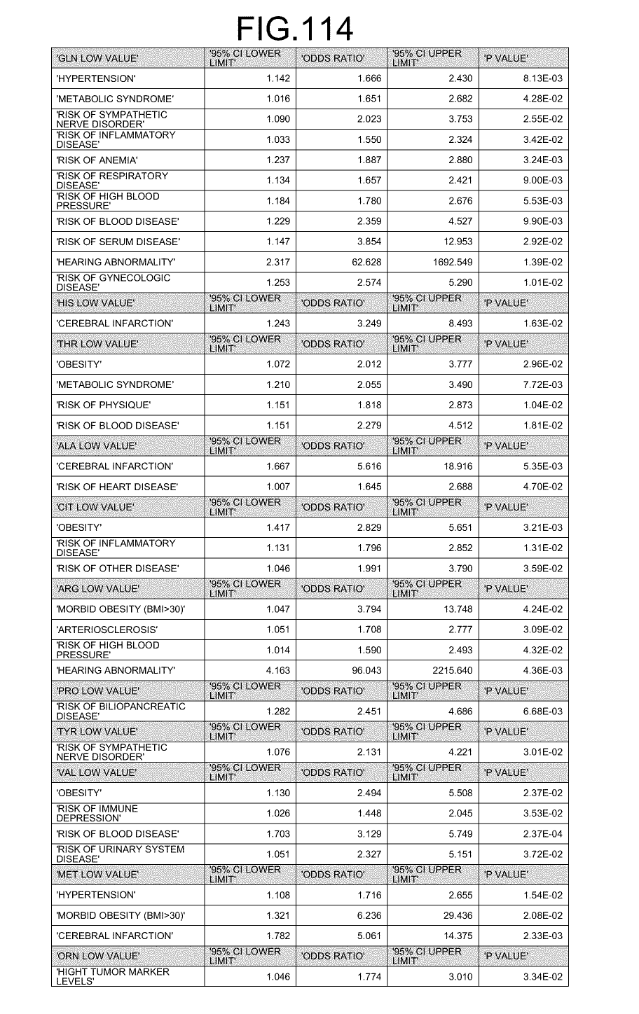

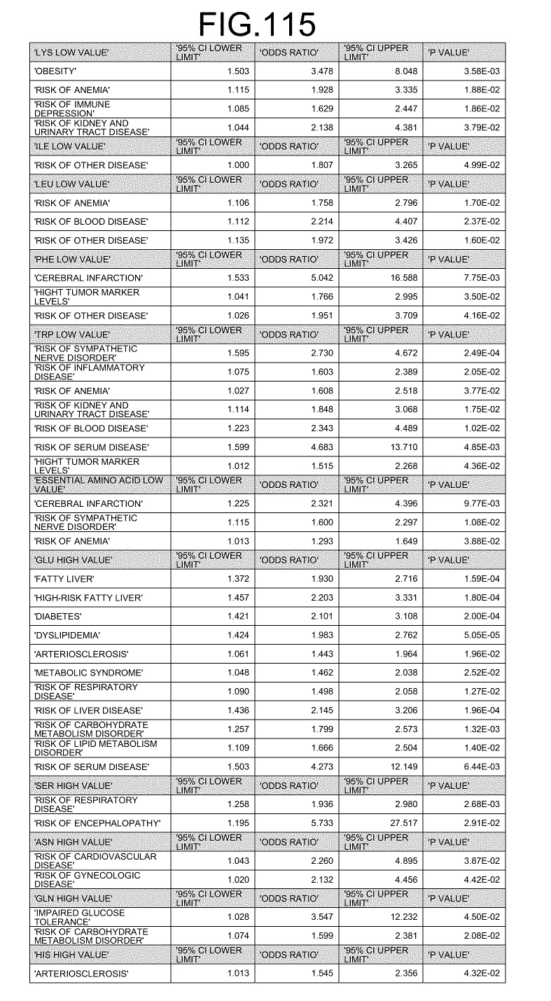

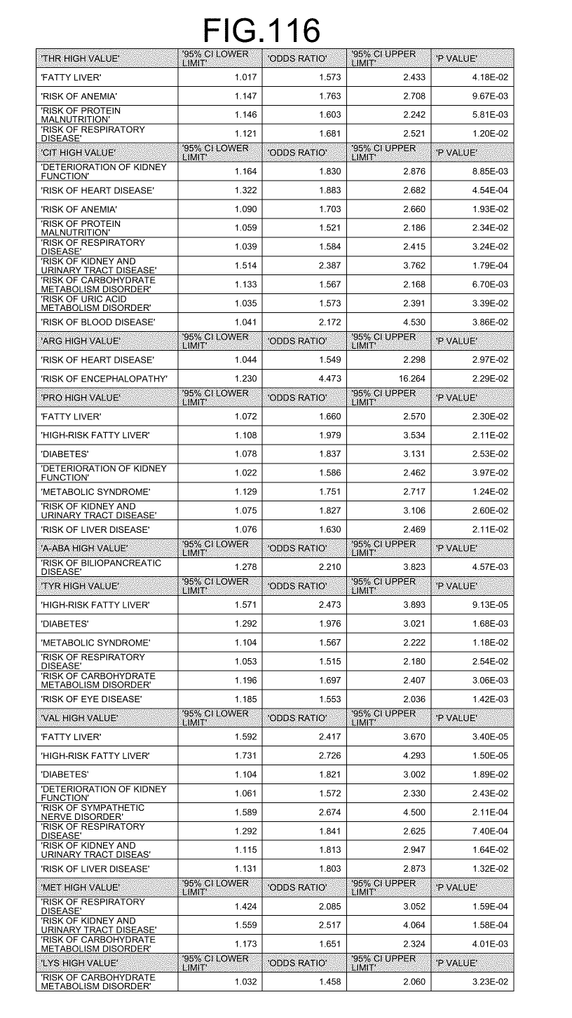

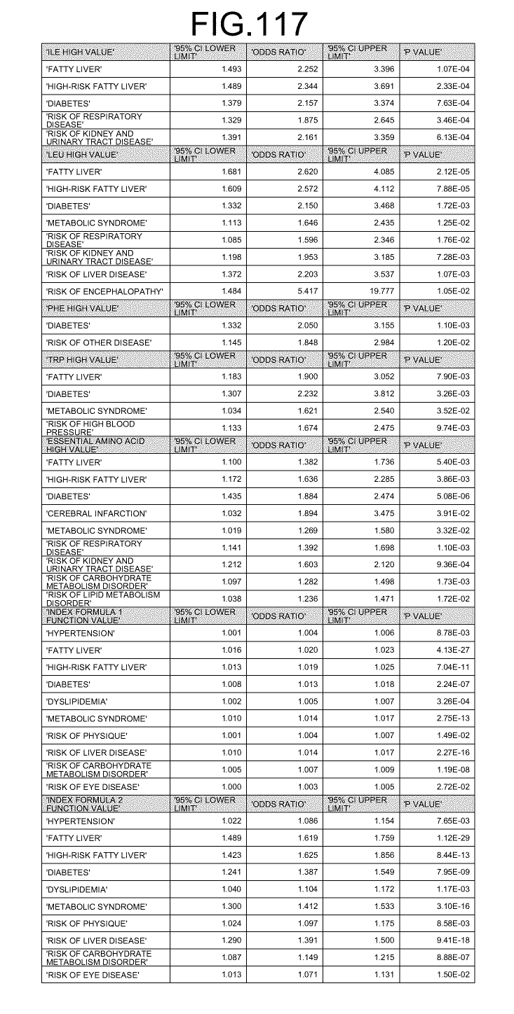

In FIG. 109 to FIG. 117, odds ratios when gender, age, and BMI as background factors are adjusted, 95%-confidence interval lower limits and 95%-confidence interval upper limits thereof, and p values (p<0.05) of the odds ratios are each listed.

As shown in FIG. 20 to FIG. 117, the fact that concentrations of many amino acids and values of Index Formulae 1 and 2 are significantly related with the presence of risk of the lifestyle-related diseases described above was confirmed, which indicates that risk of lifestyle-related diseases to humans can be evaluated in an individualized manner by comprehensively utilizing these pieces of information.

### Example 4

Background data of examinees measured in health screening and data of amino acid concentrations in blood samples taken in the health screening were obtained (in total, 7685 people). To normally distribute and standardize the amino acid concentrations in blood, the following method was conducted. To begin with, from 7685 health screening examinees (4694 males, 2991 females), a reference population of 1890 people (901 males, 989 females) are selected based on exclusion criteria based on the research article of Yamamoto, et al. (Ann Clin Biochem, 0004563215583360, first published on Mar. 31, 2015). Specifically, the reference population is determined by excluding (1) persons regularly receiving drug therapy for chronic disease, (2) persons diagnosed as being in an abnormal level, or having anemia or inflammation in medical checkups (specifically, persons matching at least one of the following conditions on test values), and (3) persons whose amino acid concentration in plasma recorded a high value or a low value by four standard deviations (SD) or more. Distributions of amino acid concentration data by gender of these 1890 people are obtained as follows.

The test values of Alb are smaller than 4.1 g/dl or greater than 5.1 g/dl.
The test values of Hb are smaller than 13.5 g/dl or greater than 16.9 g/dl for males, and are smaller than 11.0 g/dl or greater than 14.8 g/dl for females.
The test values of MCV are smaller than 82 fl or greater than 98 fl.
The test values of UA are smaller than 3.8 mg/dl or greater than 8.0 mg/dl for males, and are smaller than 2.6 mg/dl or greater than 5.6 mg/dl for females.
The test values of TG are smaller than 42 mg/dl or greater than 222 mg/dl for males, and are smaller than 30 mg/dl or greater than 124 mg/dl for females.
The test values of Glucose are smaller than 76 mg/dl or greater than 106 mg/dl.
The test values of γGT are smaller than 9 U/L or greater than 55 U/L.
The test values of ALT are smaller than 8 U/L or greater than 33 U/L.
The test values of CK are smaller than 61 U/L or greater than 257 U/L for males, and are smaller than 43 U/L or greater than 157 U/L for females.
The test values of CRP are greater than 1.4 mg/dl.
The test values of BMI are equal to or smaller than 14, or are equal to or greater than 30.

TABLE 3

| | Male Lower | Male | Male Upper |
|---|---|---|---|
| | Limit | Median | Limit |
| | 2.5 Percentiles | 50 Percentiles | 97.5 Percentiles |
| Glu | 19.0 | 40.1 | 76.6 |
| Ser | 82.8 | 110.7 | 149.0 |
| Asn | 35.9 | 46.3 | 59.7 |
| Gly | 151.3 | 205.0 | 295.5 |
| Gln | 467.3 | 576.4 | 702.0 |
| His | 66.8 | 82.3 | 101.1 |
| Thr | 88.3 | 122.6 | 169.6 |
| Ala | 239.1 | 349.7 | 491.8 |
| Cit | 21.0 | 30.2 | 43.3 |
| Arg | 64.3 | 90.4 | 122.5 |
| Pro | 94.0 | 141.7 | 243.3 |
| a-ABA | 11.7 | 19.6 | 32.5 |
| Tyr | 47.0 | 62.7 | 84.0 |
| Val | 182.1 | 232.9 | 295.3 |
| Met | 20.2 | 27.0 | 34.2 |
| Orn | 36.2 | 52.2 | 75.3 |
| Lys | 147.2 | 192.2 | 242.4 |
| Ile | 47.4 | 65.0 | 89.9 |
| Leu | 100.9 | 129.2 | 166.7 |
| Phe | 46.6 | 59.1 | 74.9 |
| Trp | 47.4 | 60.4 | 76.9 |

(The unit is μM.)

TABLE 4

| | Female Lower | Female | Female Upper |
|---|---|---|---|
| | Limit | Median | Limit |
| | 2.5 Percentiles | 50 Percentiles | 97.5 Percentiles |
| Glu | 12.1 | 27.5 | 57.2 |
| Ser | 80.3 | 115.7 | 157.0 |
| Asn | 32.7 | 43.0 | 58.9 |
| Gly | 149.1 | 214.7 | 396.7 |
| Gln | 413.8 | 551.5 | 668.6 |
| His | 62.6 | 76.9 | 92.7 |
| Thr | 68.1 | 112.4 | 168.5 |
| Ala | 203.0 | 299.4 | 442.9 |
| Cit | 17.7 | 28.6 | 41.7 |
| Arg | 50.3 | 85.1 | 119.2 |
| Pro | 70.4 | 106.2 | 187.9 |
| a-ABA | 10.7 | 18.0 | 30.7 |
| Tyr | 39.9 | 56.2 | 74.4 |
| Val | 137.5 | 182.7 | 242.5 |
| Met | 17.0 | 22.6 | 29.6 |
| Orn | 24.8 | 45.0 | 68.4 |
| Lys | 114.9 | 171.4 | 223.1 |
| Ile | 34.9 | 47.8 | 65.5 |
| Leu | 73.5 | 97.8 | 125.3 |
| Phe | 41.9 | 52.1 | 66.3 |
| Trp | 40.9 | 53.0 | 68.0 |

(The unit is μM.)

Because the amino acid concentrations are not always normally distributed, Box-Cox transformation is performed by gender for each amino acid to transform the corresponding distribution to a normal distribution. The value λ in the Box-Cox transformation formula below is calculated by a maximum likelihood method.

$${f}_{λ} ( x ) = {\int }_{1}^{x} {t}^{λ - 1} dt + 1 = \{ \begin{matrix}log x + 1 & ( λ = 0 ) \\ \frac{{x}^{λ} - 1}{λ} + 1 & ( λ \neq 0 )\end{matrix} \qquad [ FORMULA 2 ]$$

Subsequently, the resultant distribution is converted with a mean of 50 and a standard deviation of 10, and a formula for converting an amino acid concentration to a standard score (amino acid concentration standard score) is obtained by gender for each amino acid concentration.

### Example 5

Persons who took health screening consecutive five years are targeted (4297 people). From among the targeted examinees, examinees who did not develop a disease event on the first year are extracted for each disease event shown in 1. to 28. below. The amino acid concentration standard scores are calculated for each disease event using the amino acid concentrations of the extracted examinees.

For each amino acid concentration, when the corresponding amino acid concentration standard score is lower than the mean−2SD (when the amino acid concentration standard score<30), the amino acid concentration is defined as an amino acid low value (e.g., Glu low value). When the corresponding amino acid concentration standard score is higher than mean+2SD (when the amino acid concentration standard score>70), the amino acid concentration is defined as an amino acid high value (e.g., Glu high value). Out of 10 kinds of amino acids including Arg that is a semiessential amino acid in addition to the essential amino acids (Val, Leu, Ile, Phe, His, Thr, Lys, Met, Trp), when at least one amino acid concentration standard score is lower than the mean−2SD (the amino acid concentration standard score<30), the amino acid concentration is defined as an essential amino acid low value. When at least one amino acid concentration standard score is higher than the mean+2SD (the amino acid concentration standard score>70), the amino acid concentration is defined as an essential amino acid high value.

For each of 28 kinds of disease events described below, an odds ratio for event development within four years is calculated by logistic regression. For amino acid concentration standard scores, all of odds ratios the p values of which are smaller than 0.05 when the deviation increases by 1SD are calculated. For amino acid low values, amino acid high values, essential amino acid low values, essential amino acid high values, odds ratios that are equal to or greater than one and the p values of which are smaller than 0.05, which determine whether to fall under each group, are calculated. For Index Formulae 1 and 2, odds ratios that are equal to or greater than one and the p values of which are smaller than 0.05 when the function value increases by one are calculated.

1. Hypertension
If systolic blood pressure is equal to or greater than 140 mmHg or if diastolic blood pressure is equal to or greater than 90 mmHg, hypertension is diagnosed.

2. Fatty Liver
If evidence of fatty liver is observed based on a liver-to-spleen contrast ratio in an abdominal ultrasound test, fatty liver is diagnosed.

3. High Risk Fatty Liver
If fatty liver is diagnosed and if AST (GOT) is higher than 38 U/l, high-risk fatty liver is diagnosed.

4. Diabetes
If any one of Items 1 to 3 below and Item 4 are identified, diabetes is diagnosed.

Item 1: Early morning fasting blood glucose level equal to or greater than 126 mg/dl.

Item 2: 120-minute 75 g-OGTT blood glucose level equal to or greater than 200 mg/dl.

Item 3: Casual blood glucose level equal to or greater than 200 mg/dl.

Item 4: HbA1C (JDS value) equal to or greater than 6.1% [HbA1C (international standard) equal to or greater than 6.5%].

5. Impaired Glucose Tolerance
If the 120-minute 75 g-OGTT blood glucose level is equal to or greater than 140 mg/dl and equal to or smaller than 199 mg/dl, impaired glucose tolerance is diagnosed.

6. Obesity
If “the waist is equal to or greater than 85 cm for males, equal to or greater than 90 cm for females” (guideline of visceral fat area value being equal to or greater than 100 cm²) or if “the BMI is equal to or greater than 25”, obesity is diagnosed.

7. Morbid Obesity
If the BMI is equal to or greater than 30, morbid obesity is diagnosed.

8. Dyslipidemia
If “triglyceride (TG) is equal to or greater than 150 mg/dl, if HDL cholesterol is less than 40 mg/dl, or if LDL cholesterol is equal to or greater than 140 mg/dl”, dyslipidemia is diagnosed.

9. Chronic Nephropathy
If the estimated glomerular filtration rate (eGFR) is less than 60, chronic nephropathy is diagnosed.

10. Arteriosclerosis
If evidence of hardening is observed in arteriosclerosis screening, arteriosclerosis is diagnosed.

11. Cerebral Infarction
If evidence of cerebral infarction is observed in a head MRI or an MRA test, cerebral infarction is diagnosed.

12. Risk of Heart Disease
If Minnesota code falls out of the normal range, the presence of risk of heart disease is diagnosed.

13. Metabolic Syndrome
If Item 1 below is applicable, when at least two of Items 2 to 4 below are applicable, metabolic syndrome is diagnosed.

Item 1: “Waist equal to or greater than 85 cm for males, equal to or greater than 90 cm for females” (guideline for the visceral fat area value equal to or greater than 100 cm²) or “BMI equal to or greater than 25”.

Item 2: “Triglyceride equal to or greater than 150 mg/dl” and/or “HDL cholesterol less than 40 mg/dl”.

Item 3: “Systolic blood pressure equal to or greater than 130 mmHg” and/or “diastolic blood pressure equal to or greater than 85 mmHg”.

Item 4: “Fasting blood glucose equal to or greater than 110 mg/dl”.

14. Risk of Sympathetic Nerve Disorder

If the heart rate is equal to or higher than 90/min, or if the neutrophil ratio is equal to or higher than 79%, the presence of risk of sympathetic nerve disorder is diagnosed.

15. Risk of Inflammatory Disease

If the CRP value is equal to or larger than 0.3 mg/dl, the presence of risk of inflammatory disease is diagnosed.

16. Risk of Anemia

For males, if the hemoglobin content is equal to or lower than 13.5 g/dl, if the hematocrit value is equal to or smaller than 39.8%, or if the red blood cell count is equal to or lower than 427×10⁴/mm³, the presence of risk of anemia is diagnosed. For females, if the hemoglobin content is equal to or lower than 11.3 g/dl, if the hematocrit value is equal to or smaller than 33.4%, if the red blood cell count is equal to or lower than 376×10⁴/mm³, or if serum iron is equal to or less than 48 μg/dl, the presence of risk of anemia is diagnosed.

17. Risk of Protein Malnutrition

If blood albumin is less than 4 mg/dl, or total blood protein is less than 6.7 mg/dl, the presence of risk of protein malnutrition is diagnosed.

18. Risk of Immune Depression

If the lymphocyte ratio is equal to or lower than 25%, the presence of risk of immune depression is diagnosed.

19. Myocardial Infarction

If evidence of myocardial infarction is observed in the test result of electrocardiogram, the presence of myocardial infarction is diagnosed.

20. Atrial Fibrillation

If evidence of atrial fibrillation is observed in the test result of electrocardiogram, the presence of atrial fibrillation is diagnosed.

21. Extrasystole

If evidence of extrasystole is observed in the test result of electrocardiogram, the presence of extrasystole is diagnosed.

22. Arrhythmia

If evidence of atrial fibrillation or extrasystole is observed in the test result of electrocardiogram, the presence of arrhythmia is diagnosed.

23. Risk of High Blood Pressure

If the diagnostic result of this item in health screening is “careful attention is required in daily life”, “treatment is required”, “thorough examination is required”, or “treatment continues”, the presence of risk is diagnosed.

24. Risk of Kidney And Urinary Tract Disease

If the diagnostic result of this item in health screening is “careful attention is required in daily life”, “treatment is required”, “thorough examination is required”, or “treatment continues”, the presence of risk is diagnosed.

25. Risk of Biliopancreatic Disease

If the diagnostic result of this item in health screening is “careful attention is required in daily life”, “treatment is required”, “thorough examination is required”, or “treatment continues”, the presence of risk is diagnosed.

26. Risk of Urinary System Disease

If the diagnostic result of this item in health screening is “careful attention is required in daily life”, “treatment is required”, “thorough examination is required”, or “treatment continues”, the presence of risk is diagnosed.

27. Height Tumor Marker Levels

If the diagnostic result of this item in health screening is “careful attention is required in daily life”, “treatment is required”, “thorough examination is required”, or “treatment continues”, the presence of risk is diagnosed.

28. Risk of Encephalopathy

If the diagnostic result of this item in health screening is “careful attention is required in daily life”, “treatment is required”, “thorough examination is required”, or “treatment continues”, the presence of risk is diagnosed.

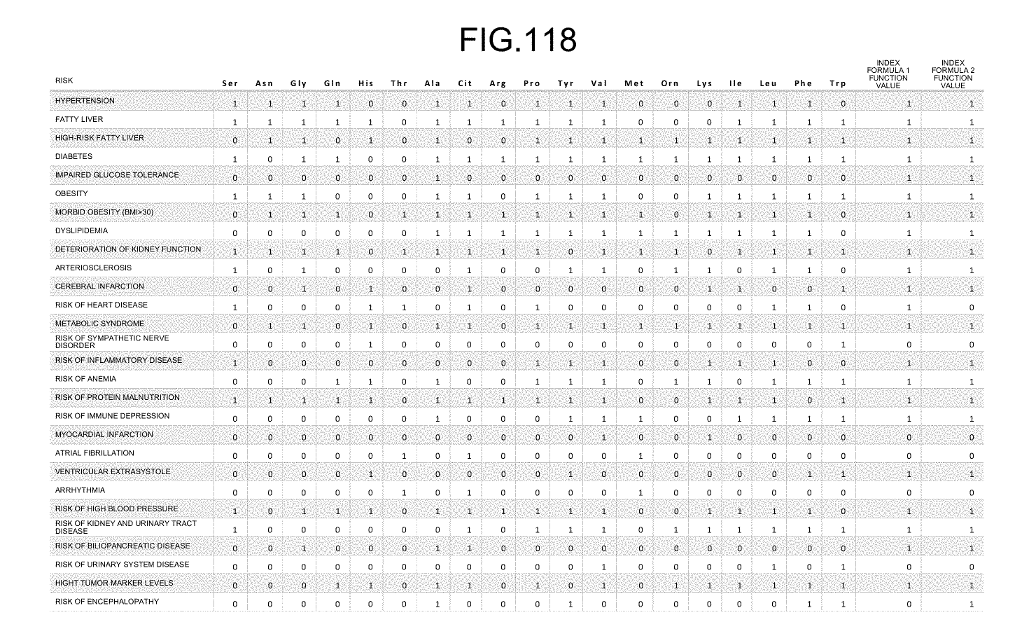

FIG. 118 shows results on whether the condition that “the p value of the odds ratio is smaller than 0.05 in at least one of the following eight occasions” is satisfied (0: unsatisfied, 1: satisfied) for each combination of amino acid concentration standard scores, values of Index Formulae 1 and 2, and the 28 kinds of disease events.
When the background factor is not adjusted.
When gender as a background factor is adjusted.
When age as a background factor is adjusted.
When BMI as a background factor is adjusted.
When gender and age as background factors are adjusted.
When gender and BMI as background factors are adjusted.
When age and BMI as background factors are adjusted.
When gender, age, and BMI as background factors are adjusted.

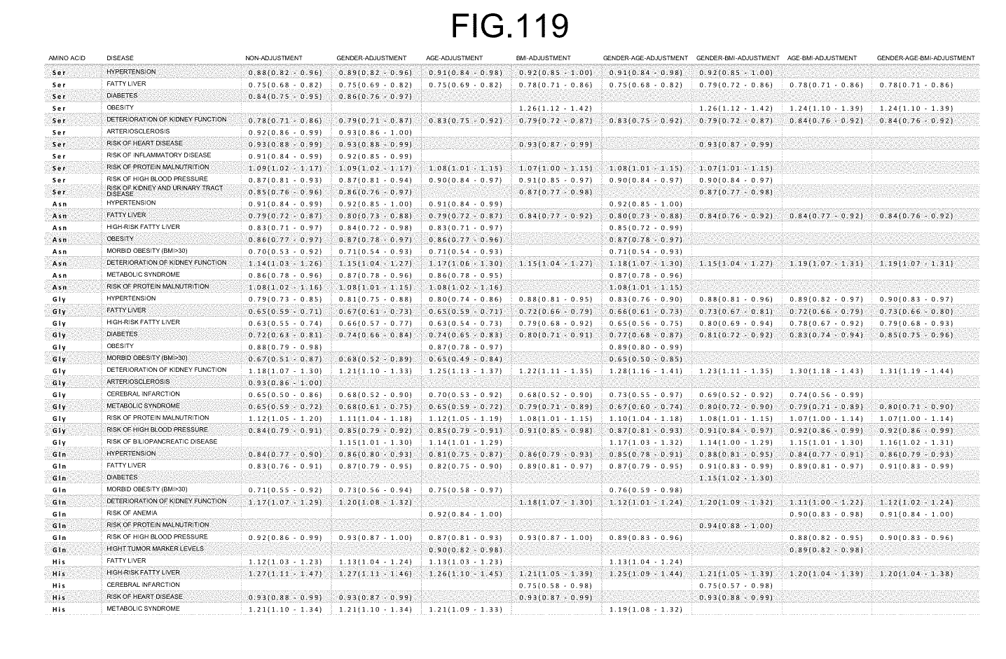

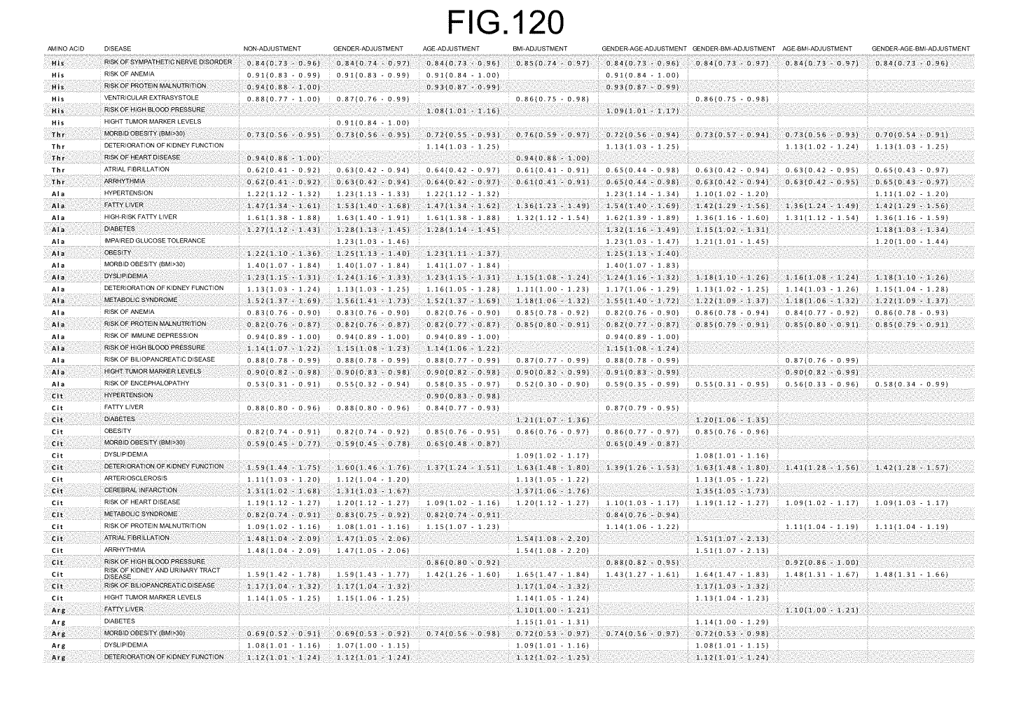

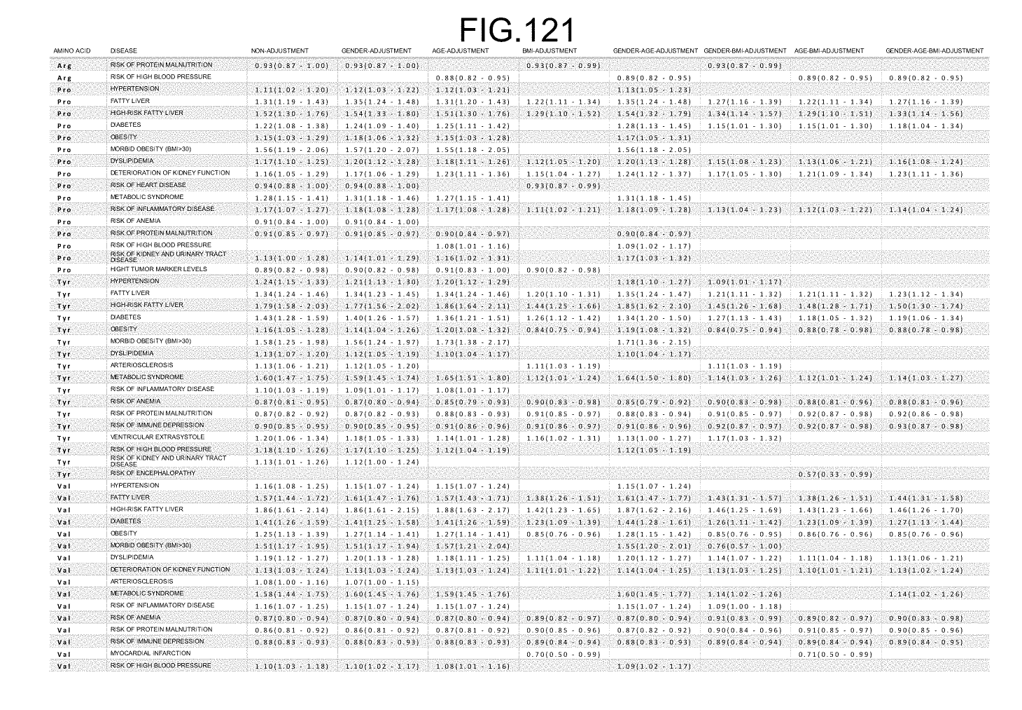

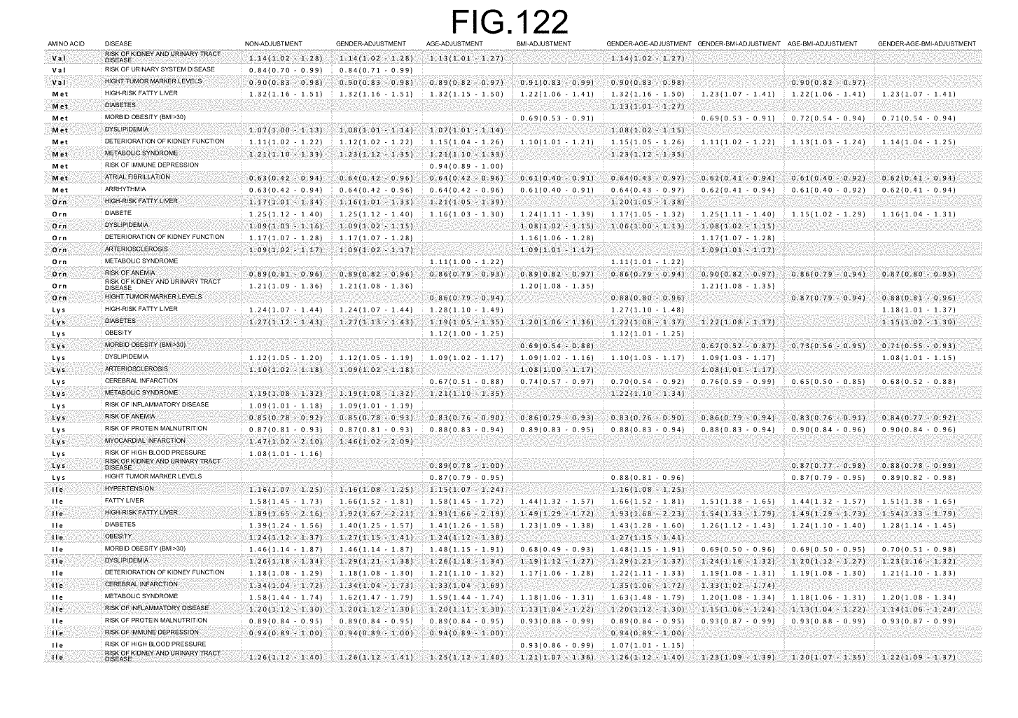

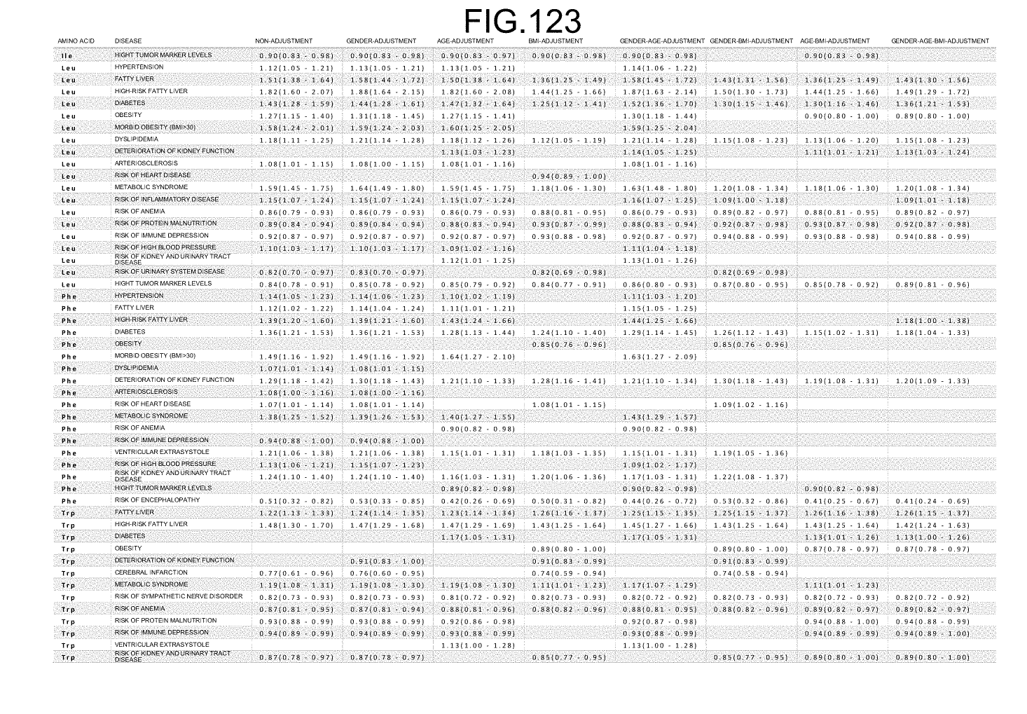

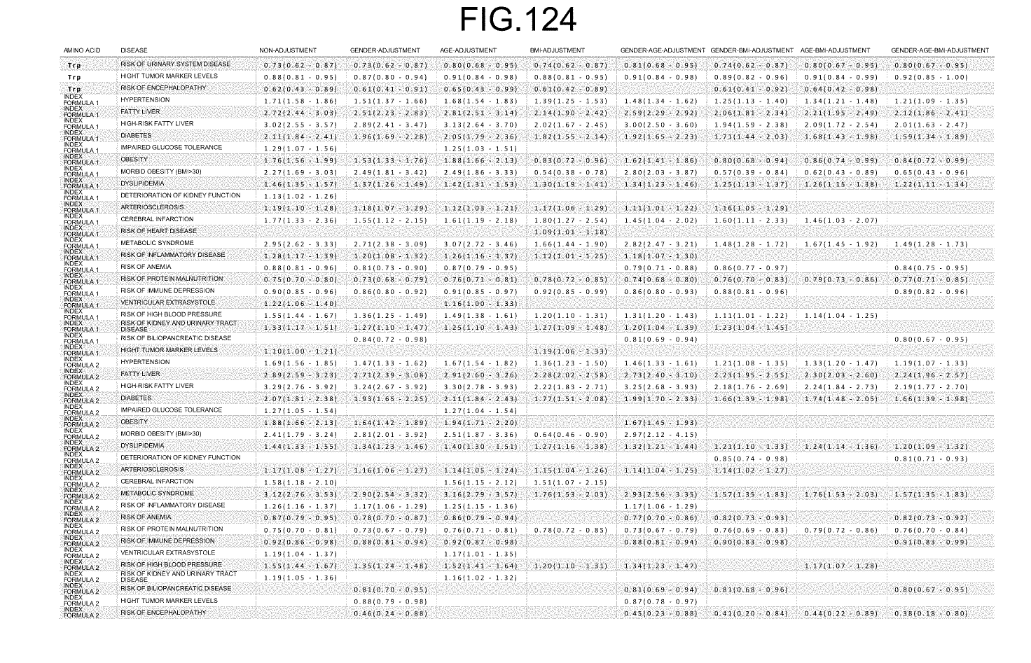

In FIG. 119 to FIG. 124, odds ratios and 95%-confidence intervals thereof (upper limits and lower limits) are each listed for combinations in which the p values of odds ratios are smaller than 0.05 out of the respective combinations of amino acid concentration standard scores, values of Index Formulae 1 and 2, and the 28 kinds of disease events. The respective numerical values listed in FIG. 119 to FIG. 124 are values each corresponding to the eight occasions.

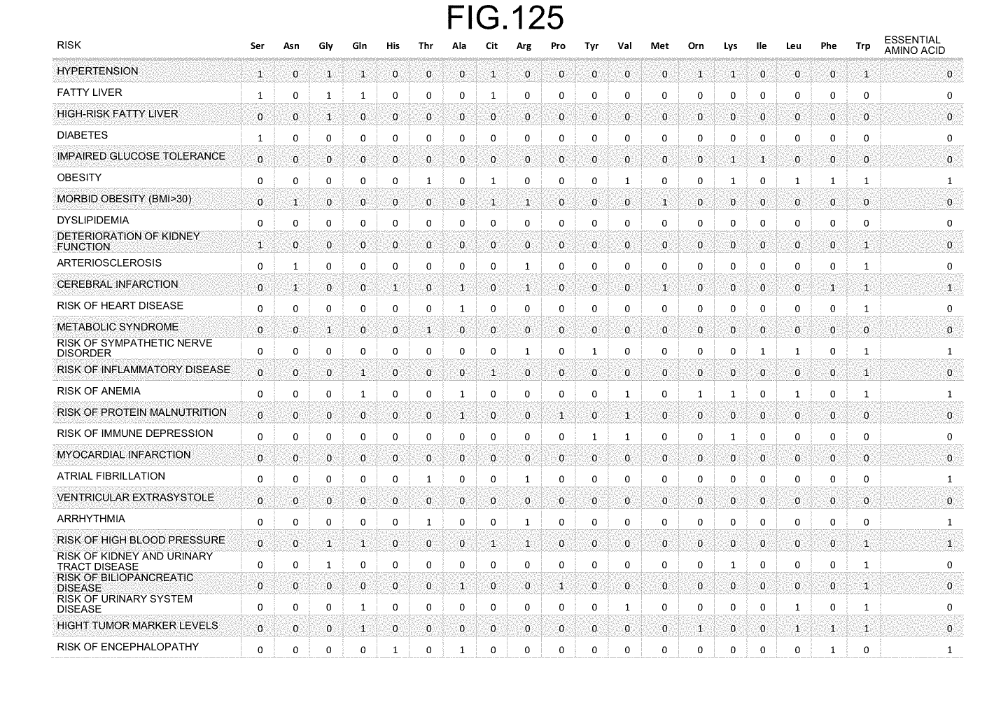

FIG. 125 shows results on whether the condition that “the p value of the odds ratio is smaller than 0.05 and the value of the odds ratio is greater than one in at least one of the eight occasions” is satisfied (0: unsatisfied, 1: satisfied) for each combination of amino acid concentration standard scores corresponding to amino acid low values, amino acid concentration standard scores corresponding to essential amino acid low values, and the 28 kinds of disease events.

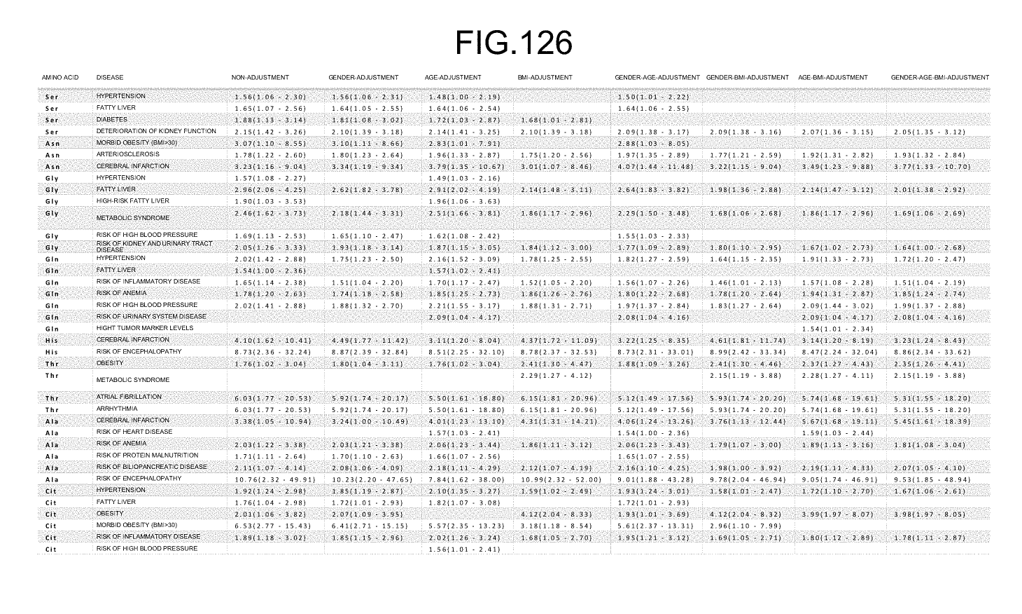

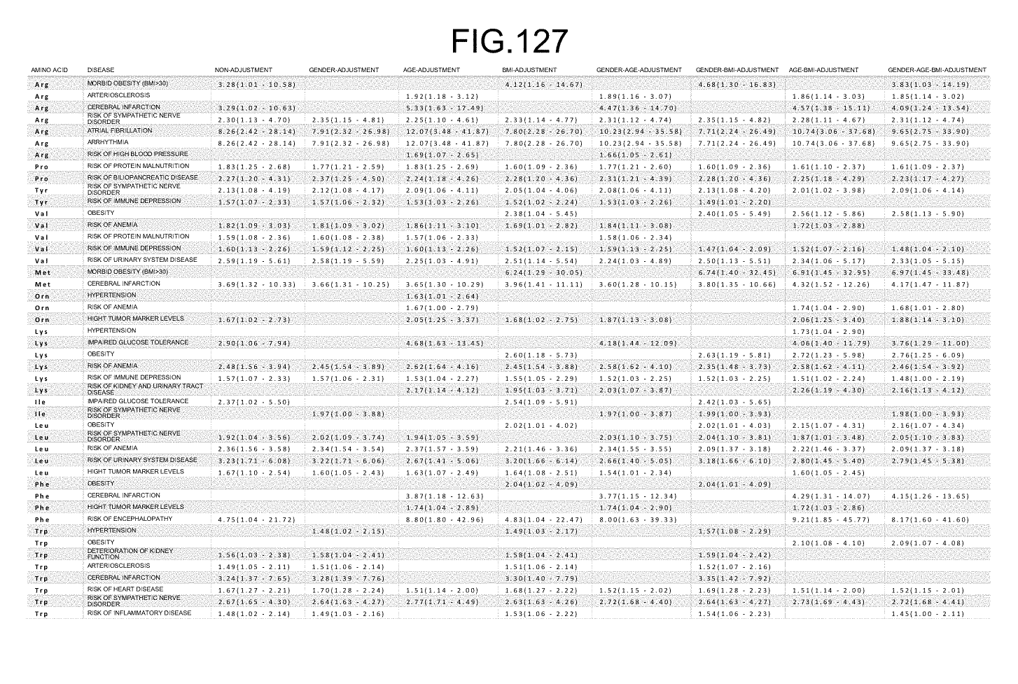

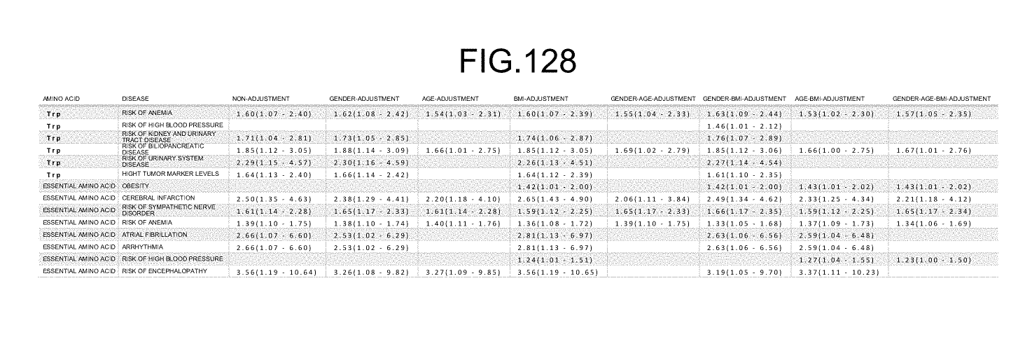

In FIG. 126 to FIG. 128, odds ratios and 95%-confidence intervals thereof (upper limits and lower limits) are each listed for combinations in which the p values of odds ratios are smaller than 0.05 and the values of the odds ratios are greater than one out of the respective combinations of amino acid concentration standard scores corresponding to amino acid low values, amino acid concentration standard scores corresponding to essential amino acid low values, and the 28 kinds of disease events. The respective numerical values listed in FIG. 126 to FIG. 128 are values each corresponding to the eight occasions.

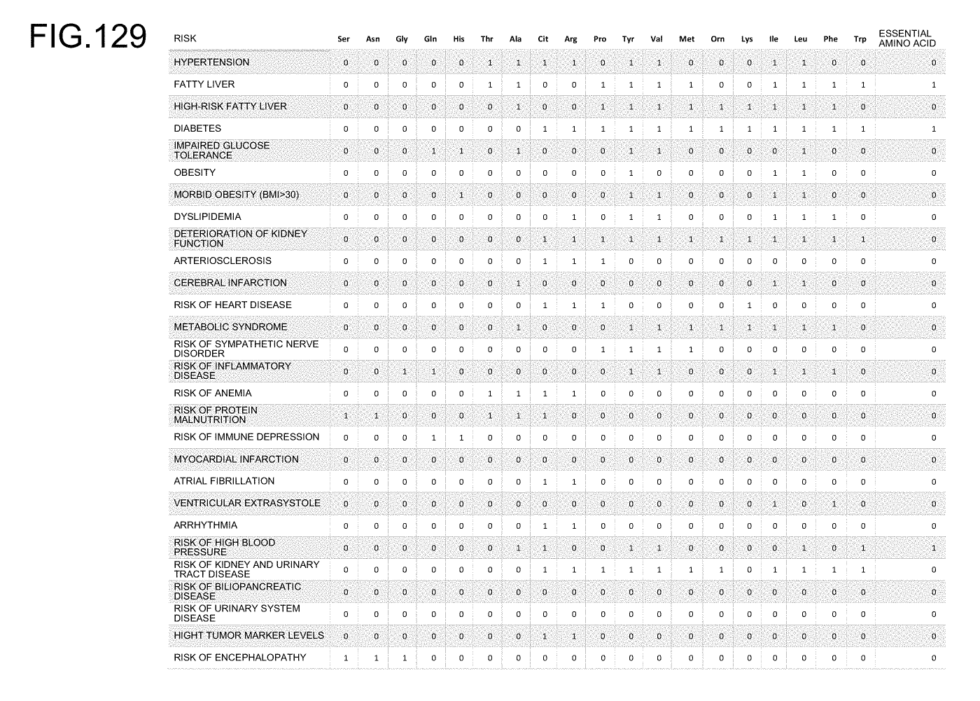

FIG. 129 shows results on whether the condition that “the p value of the odds ratio is smaller than 0.05 and the value of odds ratio is greater than one in at least one of the eight occasions” is satisfied (0: unsatisfied, 1: satisfied) for each combination of amino acid concentration standard scores corresponding to amino acid high values, amino acid concentration standard scores corresponding to essential amino acid high values, and the 28 kinds of disease events.

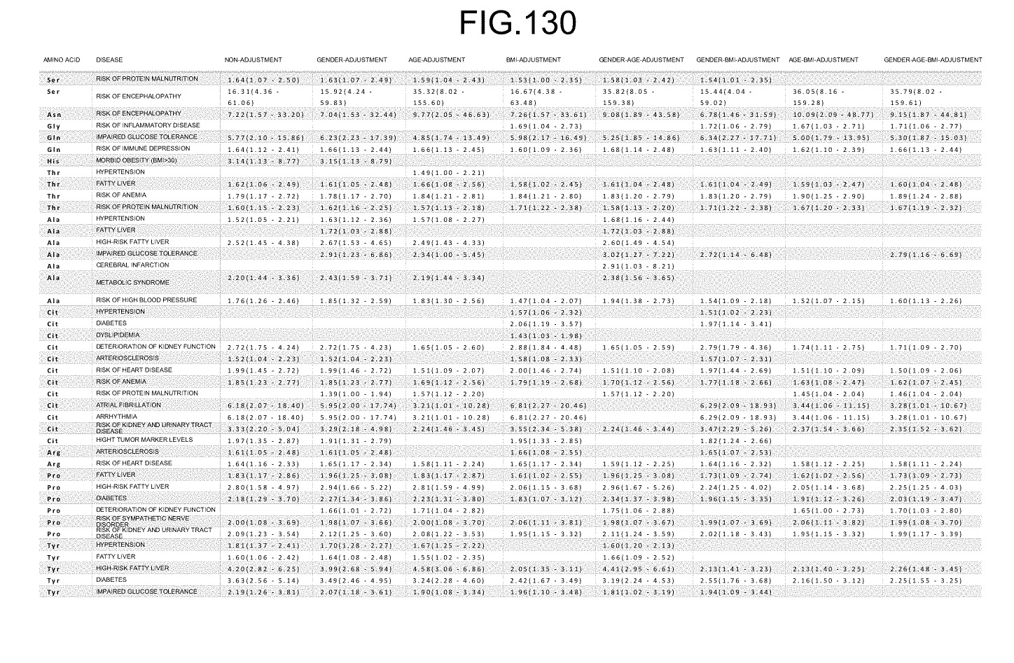

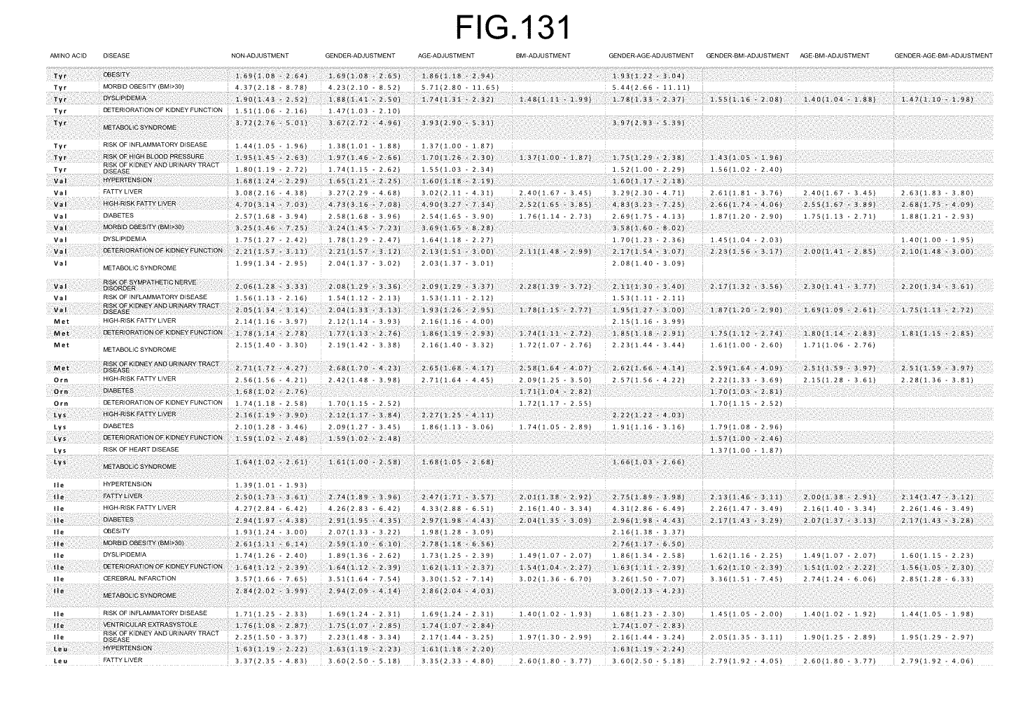

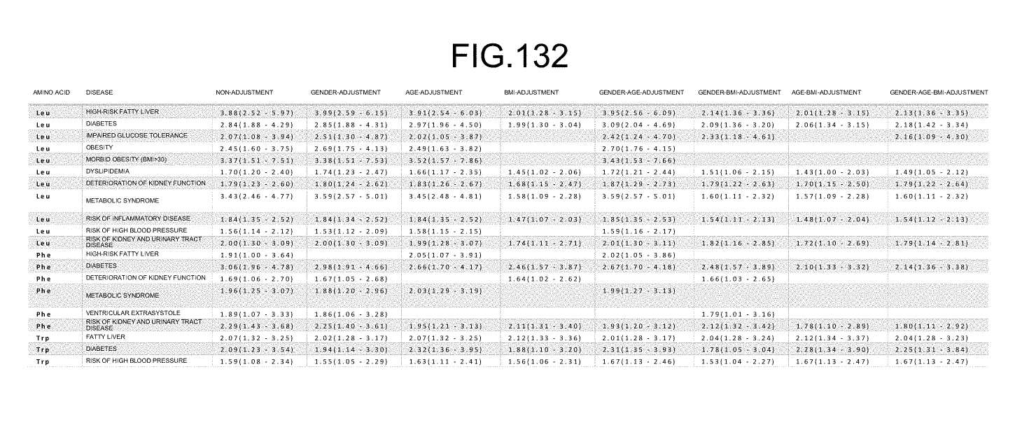

In FIG. 130 to FIG. 132, odds ratios and 95%-confidence intervals thereof (upper limits and lower limits) are each listed for combinations in which the p values of odds ratios are smaller than 0.05 and the values of the odds ratios are greater than one out of the respective combinations of amino acid concentration standard scores corresponding to amino acid high values, amino acid concentration standard scores corresponding to essential amino acid high values, and the 28 kinds of disease events. The respective numerical values listed in FIG. 130 to FIG. 132 are values each corresponding to the eight occasions.

As shown in FIG. 118 to FIG. 132, the fact that concentrations of many amino acids and values of Index Formulae 1 and 2 are significantly related with the presence of risk of the lifestyle-related diseases described above is confirmed, which indicates that risk of lifestyle-related diseases to humans can be evaluated in an individualized manner by comprehensively utilizing these pieces of information.

### Example 6

Persons who took health screening consecutive five years are targeted (4297 people). From among the targeted examinees, examinees who did not develop a disease event on the first year are extracted for each disease event caused by metabolic syndrome shown in 1. to 24. below. For each disease event, the presence of development of the disease from the first year on is used as an objective explanatory variable. The concentrations of the 19 kinds of amino acids are used as explanatory variables, and models are selected based on Cox regression with two or three amino acid explanatory variables used by using the explanatory variable coverage method. Furthermore, a function value in the obtained Cox regression equation is used as an explanatory variable, and the odds ratio corresponding to an increase of one standard deviation of this function value is calculated by logistic regression using age and gender of each subject as covariates. Models in which age and gender adjusted odds ratios thus obtained are significant when “p<0.05” are extracted for each disease event.

1. Hypertension
If systolic blood pressure is equal to or greater than 140 mmHg or if diastolic blood pressure is equal to or greater than 90 mmHg, hypertension is diagnosed.

2. Fatty Liver
If evidence of fatty liver is observed based on a liver-to-spleen contrast ratio in an abdominal ultrasound test, fatty liver is diagnosed.

3. High Risk Fatty Liver
If fatty liver is diagnosed and if AST (GOT) is higher than 38 U/l, high-risk fatty liver is diagnosed.

4. Diabetes
If any one of Items 1 to 3 below and Item 4 are identified, diabetes is diagnosed.

Item 1: Early morning fasting blood glucose level equal to or greater than 126 mg/dl.

Item 2: 120-minute 75 g-OGTT blood glucose level equal to or greater than 200 mg/dl.

Item 3: Casual blood glucose level equal to or greater than 200 mg/dl.

Item 4: HbA1C (JDS value) equal to or greater than 6.1% [HbA1C (international standard) equal to or greater than 6.5%].

5. Impaired Glucose Tolerance
If the 120-minute 75 g-OGTT blood glucose level is equal to or greater than 140 mg/dl and equal to or smaller than 199 mg/dl, impaired glucose tolerance is diagnosed.

6. Obesity
If “the waist is equal to or greater than 85 cm for males, equal to or greater than 90 cm for females” (guideline of visceral fat area value being equal to or greater than 100 cm²) or if “the BMI is equal to or greater than 25”, obesity is diagnosed.

7. Morbid Obesity
If the BMI is equal to or greater than 30, morbid obesity is diagnosed.

8. Dyslipidemia
If “triglyceride (TG) is equal to or greater than 150 mg/dl, if HDL cholesterol is less than 40 mg/dl, or if LDL cholesterol is equal to or greater than 140 mg/dl”, dyslipidemia is diagnosed.

9. Chronic Nephropathy
If the estimated glomerular filtration rate (eGFR) is less than 60, chronic nephropathy is diagnosed.

10. Arteriosclerosis
If evidence of hardening is observed in arteriosclerosis screening, arteriosclerosis is diagnosed.

11. Cerebral Infarction
If evidence of cerebral infarction is observed in a head MRI or an MRA test, cerebral infarction is diagnosed.

12. Risk of Heart Disease
If Minnesota code falls out of the normal range, the presence of risk of heart disease is diagnosed.

13. Metabolic Syndrome
If Item 1 below is applicable, when at least two of Items 2 to 4 below are applicable, metabolic syndrome is diagnosed.

Item 1: “Waist equal to or greater than 85 cm for males, equal to or greater than 90 cm for females” (guideline for the visceral fat area value equal to or greater than 100 cm²) or “BMI equal to or greater than 25”.

Item 2: “Triglyceride equal to or greater than 150 mg/dl” and/or “HDL cholesterol less than 40 mg/dl”.

Item 3: “Systolic blood pressure equal to or greater than 130 mmHg” and/or “diastolic blood pressure equal to or greater than 85 mmHg”.

Item 4: “Fasting blood glucose equal to or greater than 110 mg/dl”.

14. Risk of Physique

If the diagnostic result of this item in health screening is “careful attention is required in daily life”, “treatment is required”, “thorough examination is required”, or “treatment continues”, the presence of risk is diagnosed.

15. Risk of Cardiovascular Disease

If the diagnostic result of this item in health screening is “careful attention is required in daily life”, “treatment is required”, “thorough examination is required”, or “treatment continues”, the presence of risk is diagnosed.

16. Risk of High Blood Pressure

If the diagnostic result of this item in health screening is “careful attention is required in daily life”, “treatment is required”, “thorough examination is required”, or “treatment continues”, the presence of risk is diagnosed.

17. Risk of Kidney And Urinary Tract Disease

If the diagnostic result of this item in health screening is “careful attention is required in daily life”, “treatment is required”, “thorough examination is required”, or “treatment continues”, the presence of risk is diagnosed.

18. Risk of Liver Disease

If the diagnostic result of this item in health screening is “careful attention is required in daily life”, “treatment is required”, “thorough examination is required”, or “treatment continues”, the presence of risk is diagnosed.

19. Risk of Biliopancreatic Disease

If the diagnostic result of this item in health screening is “careful attention is required in daily life”, “treatment is required”, “thorough examination is required”, or “treatment continues”, the presence of risk is diagnosed.

20. Risk of Carbohydrate Metabolism Disorder

If the diagnostic result of this item in health screening is “careful attention is required in daily life”, “treatment is required”, “thorough examination is required”, or “treatment continues”, the presence of risk is diagnosed.

21. Risk of Lipid Metabolism Disorder

If the diagnostic result of this item in health screening is “careful attention is required in daily life”, “treatment is required”, “thorough examination is required”, or “treatment continues”, the presence of risk is diagnosed.

22. Risk of Uric Acid Metabolism Disorder

If the diagnostic result of this item in health screening is “careful attention is required in daily life”, “treatment is required”, “thorough examination is required”, or “treatment continues”, the presence of risk is diagnosed.

23. Risk of Encephalopathy

If the diagnostic result of this item in health screening is “careful attention is required in daily life”, “treatment is required”, “thorough examination is required”, or “treatment continues”, the presence of risk is diagnosed.

24. Risk of Arteriosclerosis

If the diagnostic result of this item in health screening is “careful attention is required in daily life”, “treatment is required”, “thorough examination is required”, or “treatment continues”, the presence of risk is diagnosed.

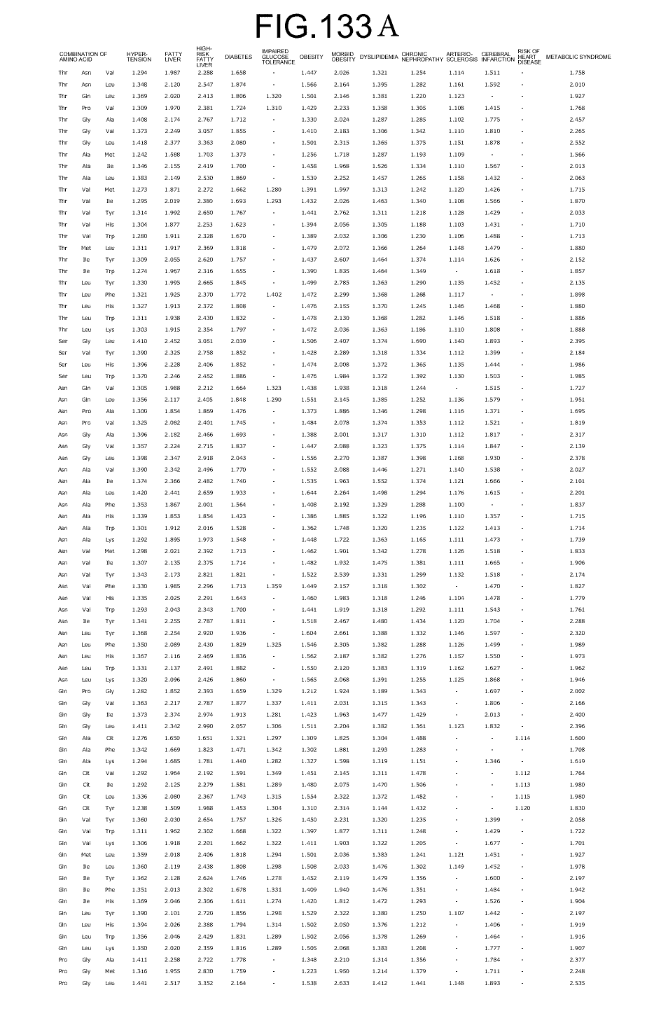

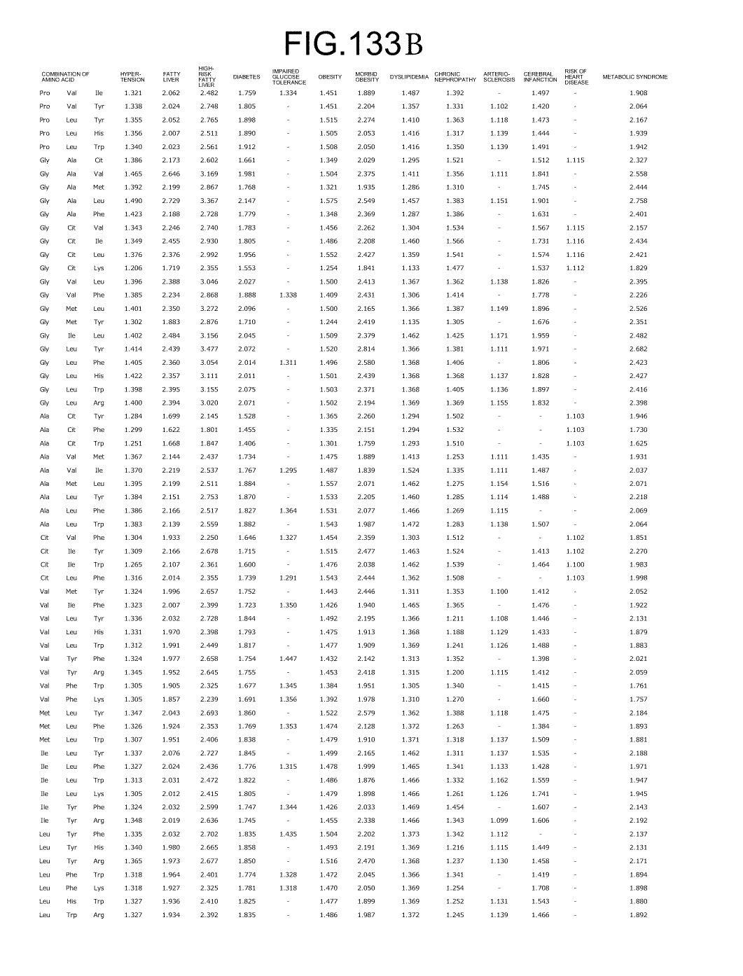

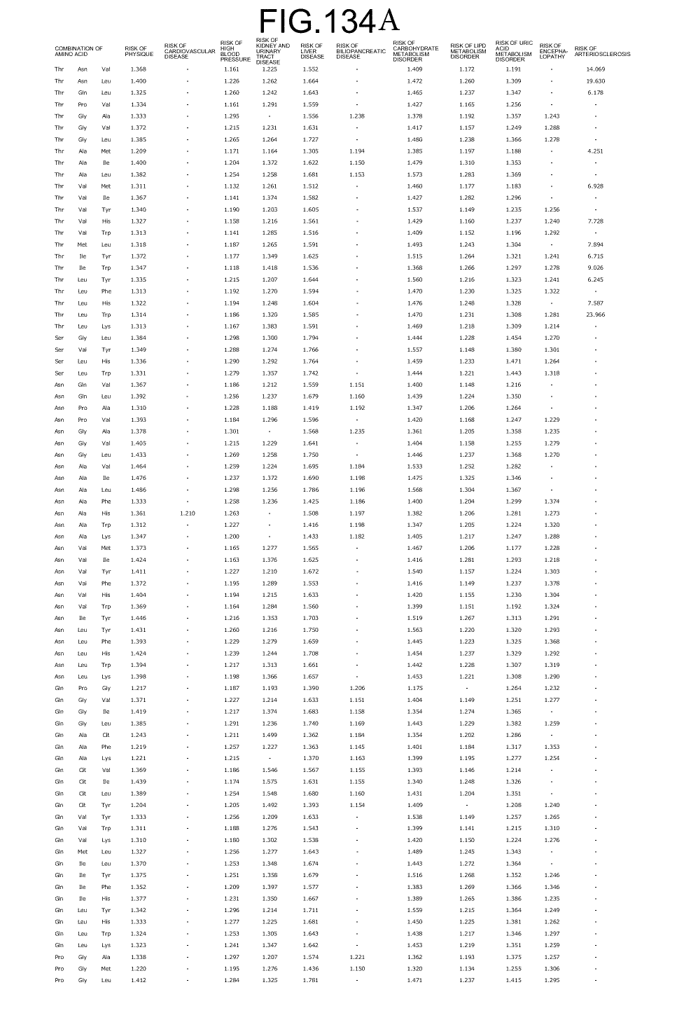

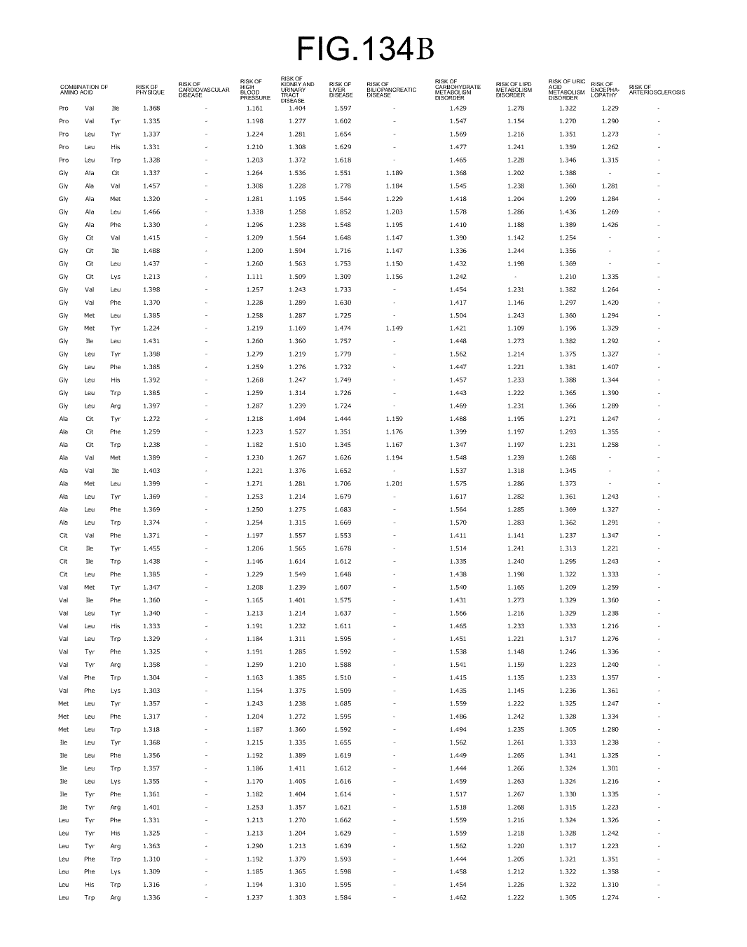

In FIG. 133A to FIG. 134B, amino acid sets each including three amino acid explanatory variables and odds ratios for a combination of the amino acid sets and disease events are listed. Each amino acid set listed in these tables satisfied the condition that the p values of odds ratios are smaller than 0.05 in combination with 19 kinds or more out of the 24 kinds of disease events when age and gender are adjusted as background factor. Odds ratios listed in the tables are those in which age and gender are adjusted.

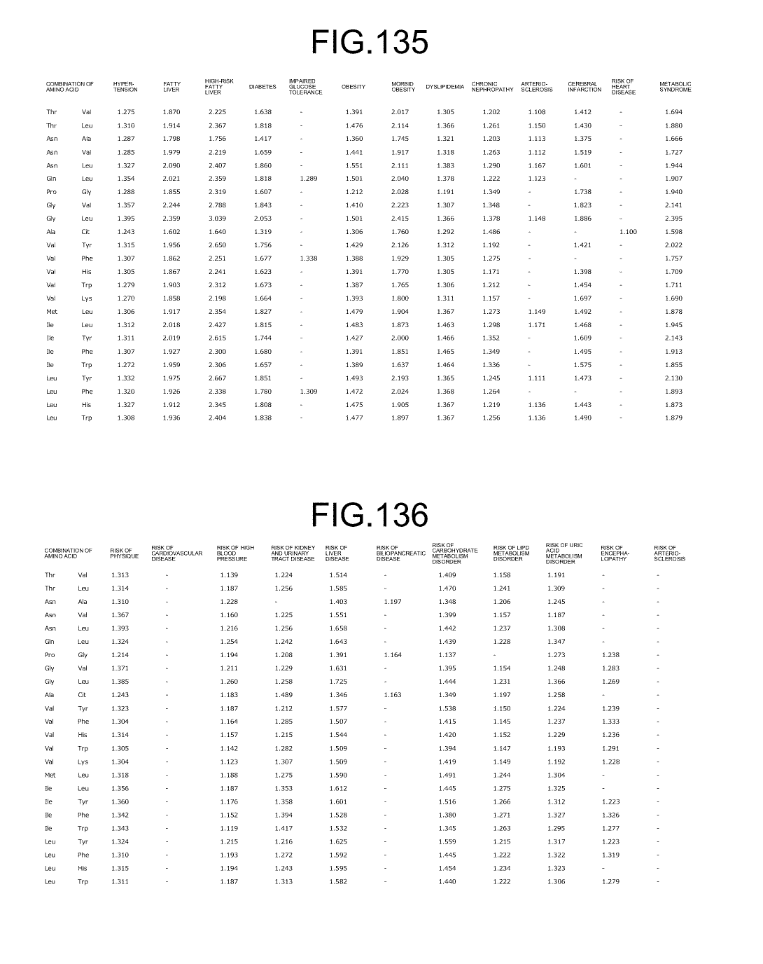

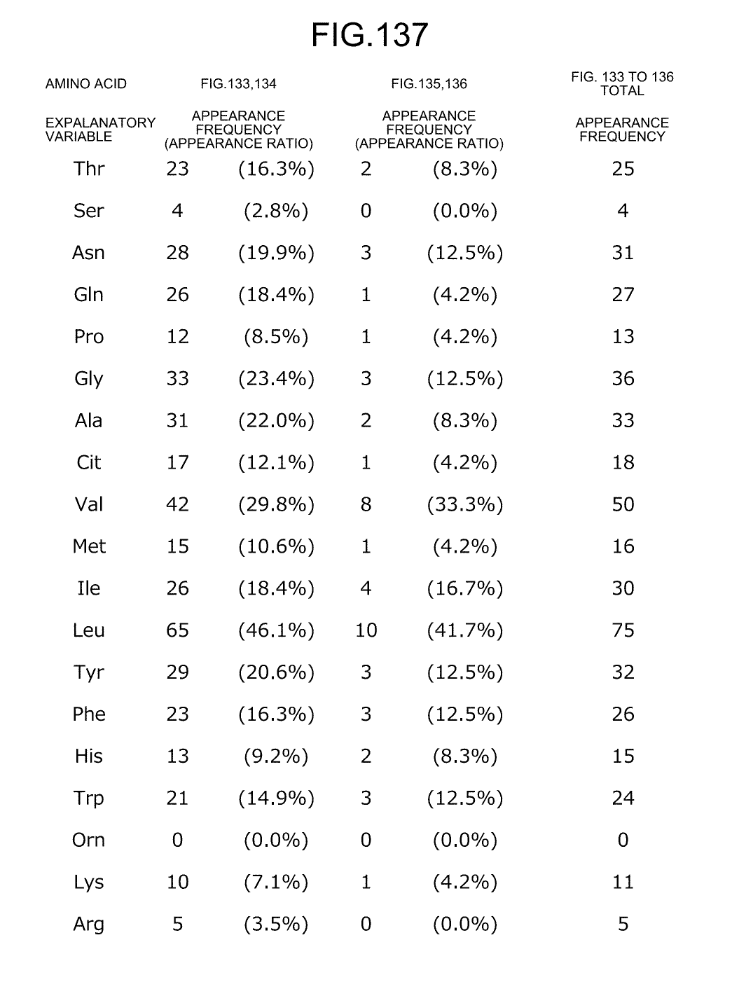

In FIG. 135 to FIG. 136, amino acid sets each including two amino acid explanatory variables and odds ratios for a combination of the amino acid sets and disease events are listed. Each amino acid set listed in these tables satisfied the condition that the p values of odds ratios are smaller than 0.05 in combination with 18 kinds or more out of the 24 kinds of disease events when age and gender are adjusted as background factor. Odds ratios listed in the tables are those in which age and gender are adjusted.

In FIG. 137, appearance frequencies in FIGS. 133 to 136 and appearance ratios in these tables for each amino acid are listed.

### Example 7

Persons who took health screening consecutive five years are targeted (4297 people). From among the targeted examinees, examinees who did not develop a disease event on the first year are extracted for each disease event caused by amino acid undernutrition shown in 1. to 8. below. For each disease event, the presence of development of the disease from the first year on is used as an objective explanatory variable. The concentrations of the 19 kinds of amino acids are used as explanatory variables, and models are selected based on Cox regression with two or three amino acid explanatory variables used by using the explanatory variable coverage method. Furthermore, a function value in the obtained Cox regression equation is used as an explanatory variable, and the odds ratio corresponding to an increase of one standard deviation of this function value is calculated by logistic regression using age and gender of each subject as covariates. Models in which age and gender adjusted odds ratio thus obtained are significant when “p<0.05” are extracted for each disease event.

1. Risk of Sympathetic Nerve Disorder

If the heart rate is equal to or higher than 90/min, or if the neutrophil ratio is equal to or higher than 79%, the presence of risk of sympathetic nerve disorder is diagnosed.

2. Risk of Inflammatory Disease

If the CRP value is equal to or larger than 0.3 mg/dl, the presence of risk of inflammatory disease is diagnosed.

3. Risk of Anemia

For males, if the hemoglobin content is equal to or lower than 13.5 g/dl, if the hematocrit value is equal to or smaller than 39.8%, or if the red blood cell count is equal to or lower than 427×10⁴/mm³, the presence of risk of anemia is diagnosed. For females, if the hemoglobin content is equal to or lower than 11.3 g/dl, if the hematocrit value is equal to or smaller than 33.4%, if the red blood cell count is equal to or lower than 376×10⁴/mm³, or if serum iron is equal to or less than 48 μg/dl, the presence of risk of anemia is diagnosed.

4. Risk of Protein Malnutrition

If blood albumin is less than 4 mg/dl, or total blood protein is less than 6.7 mg/dl, the presence of risk of protein malnutrition is diagnosed.

5. Risk of Immune Depression

If the lymphocyte ratio is equal to or lower than 25%, the presence of risk of immune depression is diagnosed.

6. Risk of Blood Disease

If the diagnostic result of this item in health screening is “careful attention is required in daily life”, “treatment is required”, “thorough examination is required”, or “treatment continues”, the presence of risk is diagnosed.

7. Risk of Serum Disease

If the diagnostic result of this item in health screening is “careful attention is required in daily life”, “treatment is required”, “thorough examination is required”, or “treatment continues”, the presence of risk is diagnosed.

8. Risk of Reduction in Bone Mineral Density

If the diagnostic result of this item in health screening is “careful attention is required in daily life”, “treatment is required”, “thorough examination is required”, or “treatment continues”, the presence of risk is diagnosed.

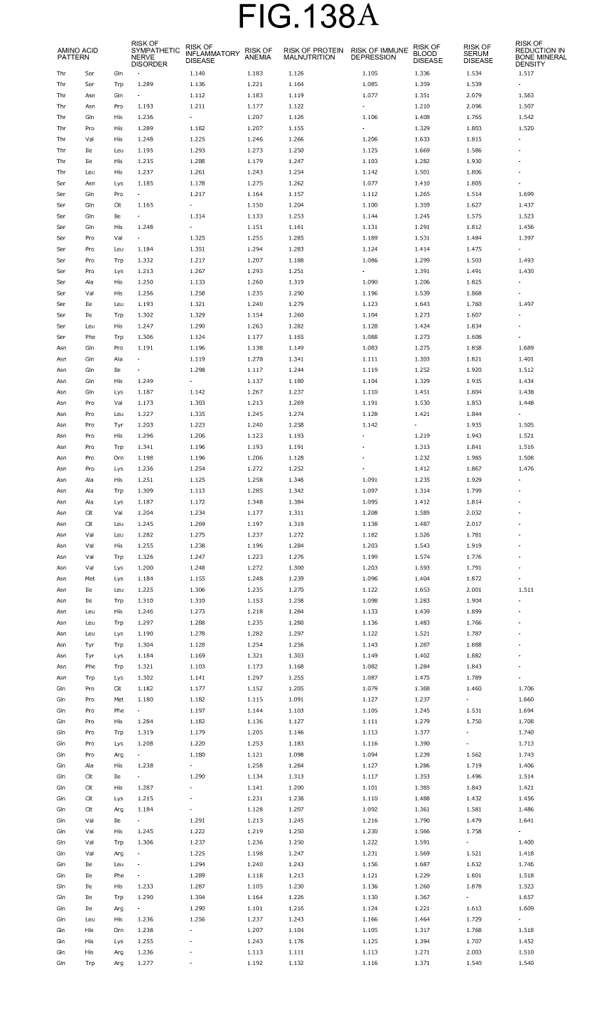

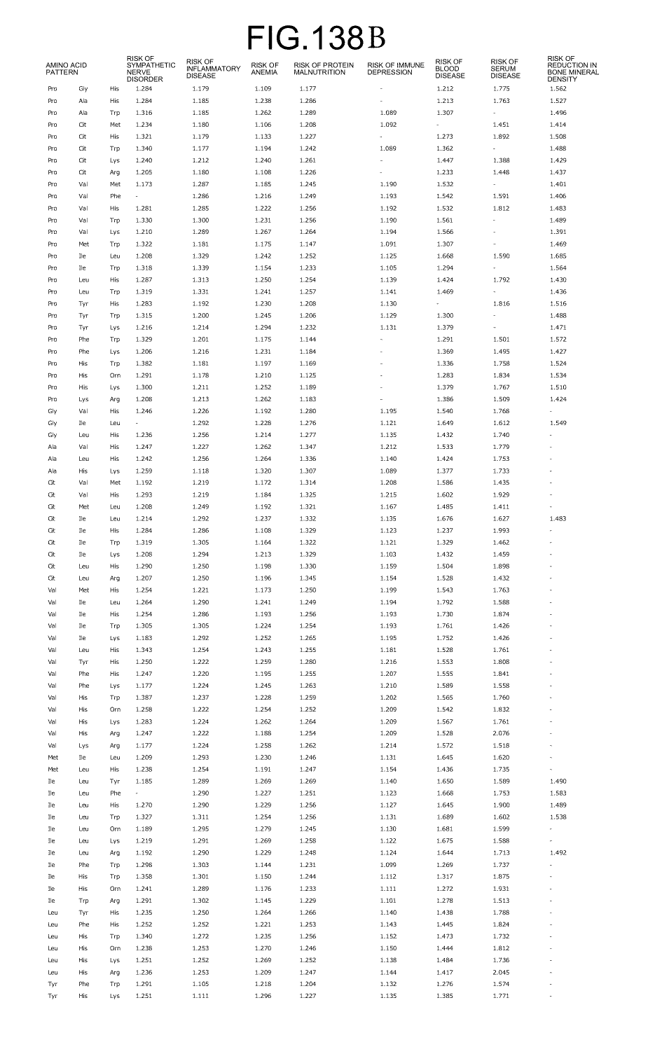

In FIG. 138A and FIG. 138B, amino acid sets each including three amino acid explanatory variables and odds ratios for a combination of the amino acid sets and disease events are listed. Each amino acid set listed in these tables satisfied the condition that the p values of odds ratios are smaller than 0.05 in combination with 7 kinds or more out of the 8 kinds of disease events when age and gender are adjusted as background factor. Odds ratios listed in the tables are those in which age and gender are adjusted.

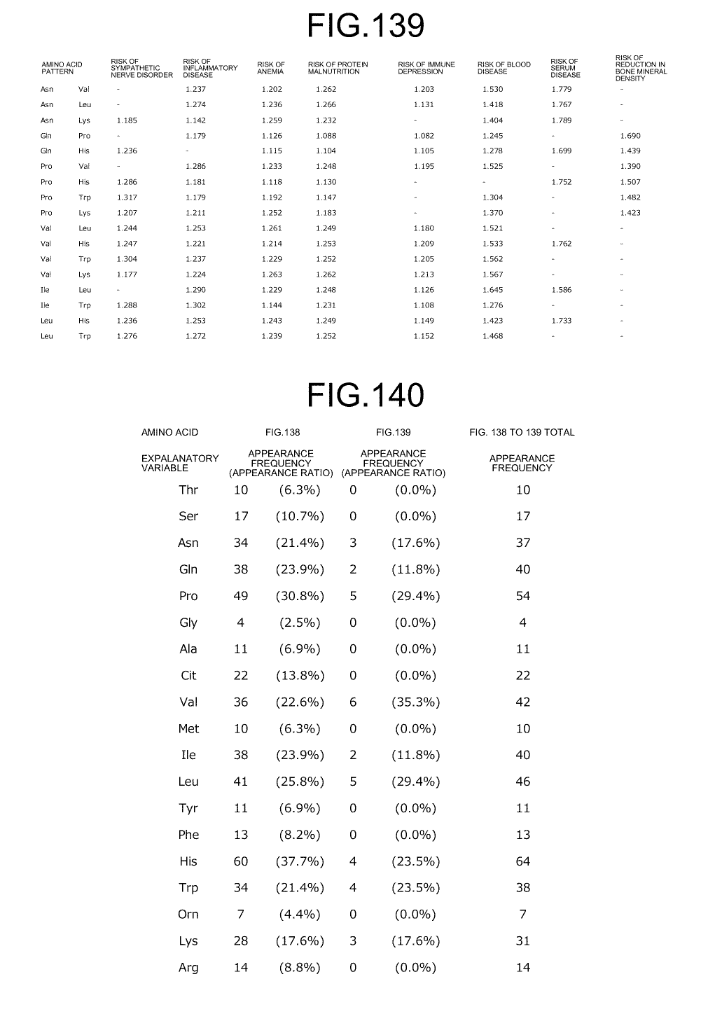

In FIG. 139, amino acid sets each including two amino acid explanatory variables and odds ratios for a combination of the amino acid sets and disease events are listed. Each amino acid set listed in these tables satisfied the condition that the p values of odds ratios are smaller than 0.05 in combination with 6 kinds or more out of the 8 kinds of disease events when age and gender are adjusted as background factor. Odds ratios listed in the tables are those in which age and gender are adjusted.

In FIG. 140, appearance frequencies in FIGS. 138A to 139 and appearance ratios in these tables for each amino acid are listed.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluating method comprising:
an evaluating step of evaluating a future risk of lifestyle-related disease for a subject to be evaluated using a concentration value of an amino acid included in amino acid concentration data on the concentration value of the amino acid in blood collected from the subject.

2. The evaluating method according to claim 1, wherein at the evaluating step, if the concentration value of an amino acid included in the amino acid concentration data or a converted value of the concentration value is smaller than a predetermined value or is equal to or smaller than the predetermined value, or the concentration value or the converted value is equal to or greater than a predetermined value or is greater than the predetermined value, the future risk of lifestyle-related disease for the subject is evaluated.

3. The evaluating method according to claim 1, wherein the amino acid concentration data includes concentration values of His, Ile, Leu, Lys, Met, Phe, Thr, Trp, Val, and Arg.

4. The evaluating method according to claim 3, wherein at the evaluating step, if a concentration value or a converted value of the concentration value of at least one amino acid of His, Ile, Leu, Lys, Met, Phe, Thr, Trp, Val, and Arg is smaller than a predetermined value or is equal to or smaller than the predetermined value, or the concentration value or the converted value is equal to or greater than a predetermined value or is greater than the predetermined value, a future risk of developing at least one of cerebral infarction, anemia, atrial fibrillation, and arrhythmia for the subject is evaluated.

5. The evaluating method according to claim 2, wherein at the evaluating step, at least one of (1) evaluation of a future risk of developing anemia if a concentration value or a converted value of the concentration value of at least one amino acid of Lys, Leu, and Trp is smaller than a predetermined value or is equal to or smaller than the predetermined value, (2) evaluation of a future risk of developing cerebral infarction if a concentration value or a converted value of the concentration value of at least one amino acid of His, Met, and Phe is smaller than a predetermined value or is equal to or smaller than the predetermined value, and (3) evaluation of a future risk of developing any one or both of atrial fibrillation and arrhythmia if a concentration value or a converted value of the concentration value of Thr or Arg is smaller than a predetermined value or is equal to or smaller than the predetermined value is performed.

6. The evaluating method according to claim 2, wherein the converted value is an amino acid concentration standard score that is a value into which a concentration value of an amino acid is standardized, and the amino acid concentration standard score is used at the evaluating step.

7. An evaluating apparatus comprising a control unit, wherein the control unit includes an evaluating unit that evaluates a future risk of lifestyle-related disease for a subject to be evaluated using a concentration value of an amino acid included in amino acid concentration data of the subject on the concentration value of the amino acid in blood.

8. An evaluating method carried out with an information processing apparatus including a control unit, wherein the evaluating method comprises an evaluating step of evaluating a future risk of lifestyle-related disease for a subject to be evaluated using a concentration value of an amino acid included in amino acid concentration data of the subject on the concentration value of the amino acid in blood, wherein the evaluating step is executed by the control unit.

9. An evaluating program product having a non-transitory computer readable medium including programmed instructions for making an information processing apparatus including a control unit execute an evaluating method, wherein the evaluating method comprises an evaluating step of evaluating a future risk of lifestyle-related disease for a subject to be evaluated using a concentration value of an amino acid included in amino acid concentration data of the subject on the concentration value of the amino acid in blood.

10. An evaluating system comprising (I) an evaluating apparatus including a control unit and (II) a terminal apparatus including a control unit to provide amino acid concentration data of a subject to be evaluated on a concentration value of an amino acid that are connected to each other communicatively via a network, wherein
the control unit of the terminal apparatus includes:
an amino acid concentration data-sending unit that transmits the amino acid concentration data of the subject to the evaluating apparatus; and
a result-receiving unit that receives an evaluation result on a future risk of lifestyle-related disease for the subject, transmitted from the evaluating apparatus, and
the control unit of the evaluating apparatus includes:
an amino acid concentration data-receiving unit that receives the amino acid concentration data of the subject transmitted from the terminal apparatus;
an evaluating unit that evaluates the future risk of lifestyle-related disease for the subject using the concentration value of the amino acid included in the amino acid concentration data of the subject received by the amino acid concentration data-receiving unit; and
a result-sending unit that transmits the evaluation result of the subject obtained by the evaluating unit to the terminal apparatus.

11. A terminal apparatus comprising a control unit, wherein the control unit includes a result-obtaining unit that obtains an evaluation result on a future risk of lifestyle-related disease for a subject to be evaluated,
wherein the evaluation result is the result of evaluating the future risk of lifestyle-related disease for the subject using a concentration value of an amino acid included in amino acid concentration data of the subject on the concentration value of the amino acid in blood.

12. An evaluating apparatus comprising a control unit, being connected communicatively via a network to a terminal apparatus that provides amino acid concentration data of a subject to be evaluated on a concentration value of an amino acid in blood, wherein the control unit includes:
an amino acid concentration data-receiving unit that receives the amino acid concentration data of the subject transmitted from the terminal apparatus;
an evaluating unit that evaluates a future risk of lifestyle-related disease for the subject using the concentration value of the amino acid included in the amino acid concentration data of the subject received by the amino acid concentration data-receiving unit; and
a result-sending unit that transmits an evaluation result obtained by the evaluating unit to the terminal apparatus.
